(19) United States
(12) Patent Application Publication
(10) Pub. No.: US 2024/0398806 A1
(43) Pub. Date: Dec. 5, 2024

(54) Compositions For and Methods of Treating and/or Preventing Pain

(71) Applicants: Duke University, Durham, NC (US); Sanford Burnham Prebys Medical Discovery Inst., La Jolla, CA (US)

(72) Inventors: Lawrence Barak, Durham, NC (US); Marc Caron (deceased), Durham, NC (US); Lauren Slosky, Durham, NC (US); Ru-Rong Ji, Durham, NC (US); Anthony Pinkerton, La Jolla, CA (US)

(21) Appl. No.: 18/560,394

(22) PCT Filed: May 13, 2022
(86) PCT No.: PCT/US22/29135

§ 371 Date: Nov. 12, 2023

Related U.S. Application Data
(60) Provisional application No. 63/188,113, filed on May 13, 2021.

(51) Int. Cl. A61K 31/517; A61K 45/06; A61P 25/04; C07D 401/04

(52) U.S. Cl. CPC A61K 31/517; A61K 45/06; A61P 25/04; C07D 401/04

(57) ABSTRACT

Disclosed herein are compositions and pharmaceutical formulations comprising a functionally selected B-arrestin-biased NTSR1 ligand (such as SBI-553 and SBI-810) and methods of using those compositions and pharmaceutical formulations for treating and/or preventing pain.

### CROSS-REFERENCE TO RELATED APPLICATIONS

This application claims priority to U.S. Provisional Application No. 63/188,113 filed 13May 2021, which is incorporated herein in its entirety.

### STATEMENT REGARDING FEDERALLY SPONSORED RESEARCH OR DEVELOPMENT

This invention was made with government support under 1UG3-DA050316-01 and 5P30-DA029925, K99-DA048970, R00-DA048970, and R01-NS87988 awarded by the National Institutes of Health and W81XWH2210267 awarded by the Department of Defense. The government has certain rights in the invention.

### BACKGROUND

Neurotensin (NT) is a brain-gut tridecapeptide that serves a dual function. NT is both a neurotransmitter and neuromodulator in the nervous system and a paracrine and circulating hormone in the periphery (Kitabgi P, et al. (2002) Curr Opin Drug Discov Devel. 5(5):764-776). Neurotensin mediates its effects manly through two GPCRs (NTSR1, and NTSR2). Their several analogs have been shown to elicit analgesia in animal models of acute and chronic pain that is both independent of opiates and in a variety of pain paradigms (Feng Y P, et al. (2015) Curr Pharm Des. 21(7):840-848; Kleczkowska P, et al. (2013) Eur J Pharmacol. 716(1-3):54-60; Smith K E, et al. (2012) Behav Brain Res. 232(1):93-97). Since opiates are at the forefront for the treatment of acute pain as well as some forms of chronic pain (Gomes T, et al. (2014) Can Fam Physician. 60(9):826-832), using neurotensin as a modality to treat acute pain (e.g., postoperative pain) and chronic pain (e.g., neuropathic pain) has the potential to bypass the long list of opiate mediated side effects like constipation, nausea, drowsiness, sleep disturbance, respiratory depression, medication compliance, as well as dependence to name a few (Bruneau J, et al. (2018) CMAJ. 190(9):E247-E257: McNicol E, et al. (2003) J Pain. 4(5):231-256).

Studies have demonstrated pharmacologically that the minimal biologically active analog of the six terminal amino acids of NT could not be reversed by the opioid antagonists naloxone and naltrexone but could provide stronger pain relief than morphine (Bredeloux P, et al. (2006) Behav Brain Res. 175(2):399-407: Nemeroff C B, et al. (1979) Proc Natl Acad Sci USA. 76(10):5368-5671). Therefore, over the past two decades. NT peptide agents have gained interests as therapeutics because of their high potency. But, their limited bioavailability due to their shorty half-life and rapid clearance has limited their development as therapeutics (Fosgerau K, et al. (2015) Drug Discov Today. 20(1):122-128).

Thus, there remains an urgent need for a non-invasive, definitive therapy to address the management of acute and chronic pain. Consequently, the present disclosure provides compositions for and methods of treating, managing, and/or preventing a subject's pain, which can be used alone or in combination with other treatments.

### BRIEF DESCRIPTION OF THE FIGURES

FIG. 1 shows the optimization of a quinazoline based lead ML314 to give SBI-553, a potent and brain penetrant NTSR1 β-arrestin biased ago-allosteric modulator in the pre-IND phase of development.

FIG. 2 shows β-arrestin biased signaling at the NTSR1.

FIG. 3 shows β-arrestin biased neurotensin modulators, wherein the use of β-arrestin-2 biased NTSR1 agonists to alleviate neuropathic pain and surgical pain without eliciting side effects associated with neurotensin peptide agonists.

FIG. 4A-FIG. 4D show SBI-553 activates NTSR1 to stimulate β-arrestin-associated cellular responses. FIG. 4A shows ^{[3}H^{]}SBI-553 saturation binding to NTSR1-containing HEK293T cell membranes. FIG. 4B shows ^{[3}H^{]}NTS saturation binding to NTSR1-containing HEK293T cell membranes in the absence (vehicle) and presence of SBI-553 (0.01 mM-10 mM).

FIG. 4C shows confocal imaging of-arrestin2 translocation to NTSR1. U2OS cells expressing NTSR1 and β-arrestin2-GFP without stimulation (Basal) and following treatment with vehicle or 30 mM of the antagonist SR142948A in combination with 10 nM NTS or 10 mM SBI-553. Scale bar, 10 mm. FIG. 4D shows brain membranes (100 μg/well) obtained from the striatum of DAT (dopamine transporter) knockout mice were incubated in the presence of either 1 μM cold NT or the indicated concentrations of ML314 at room temperature for 10 min, at which time 10 μM of ¹²⁵I-NT was added for 90 min. The ordinate axis in counts per minute (CPM) represents specifically bound ¹²⁵I-NT. In each experiment, the assays were conducted in quadruplicate, and data are expressed as the mean+SEM (n=4 separate experiments) and were analyzed using one-way ANOVA with Tukey's multiple comparison post hoc test. Curve fitting and statistical analyses were performed using GraphPad Prism V5.04.

FIG. 5A shows that unlike NT. SBI-553 cannot activate the G protein Gq and hence does not activate intracellular Ca⁺⁺ while FIG. 5B shows that SBI-553 is as effective as NT to activate the intracellular translocation of β-arrestin (right image).

FIG. 6A-FIG. 6C shows that SBI-553 treatment is not associated with hypothermia or hypotension. FIG. 6A shows animals received vehicle (0.2% DMSO in saline) or PD149163 (i.p.) at time 0). FIG. 6B shows animals received vehicle (saline) or SBI-553 (i.p.) at time 0. FIG. 6C shows systolic ventricular pressure was continuously recorded in anesthetized mice after treatment with vehicle (0.6% DMSO in saline), SBI-553, or PD149163. Baseline equals 0.

FIG. 7A-FIG. 7C shows the in vivo response of psychostimulants in the presence of SBI-553. FIG. 7A shows mice were trained to self-administer cocaine intravenously via active lever responding at a fixed lever response to reinforcement schedule (FR) of 4. Once stable performance was achieved, self-administration was assessed once daily in 60 min sessions. Animals received vehicle (saline, i.p.) or SBI-553 (2, 6, or 12 mg/kg, i.p.) immediately prior to placement into self-administration chambers with access to cocaine at doses of 0.1, 0.3, 0.5, or 1 mg/kg/infusion. FIG. 7B-FIG. 7C show cocaine-induced and methamphetamine-induced hyperlocomotion. FIG. 7B shows animals were acclimated to the open field for 30 min prior to concurrent administration of cocaine (30 mg/kg, i.p.) and SBI-553 (12 mg/kg, i.p.) or vehicle (saline, i.p.). FIG. 7C shows animals were acclimated to the open field for 30 min prior to concurrent administration of methamphetamine (2 mg/kg, i.p.) and SBI-553 (12 mg/kg, i.p.) or vehicle (saline, i.p.).

FIG. 8A-FIG. 8B shows determinations of SBI-553 PK/PD. FIG. 8A shows studies were made in mouse and rat for SBI-553, and monkey to compare an alternative compound SBI-810 to SBI-553. FIG. 8B shows graphic images show values of plasma concentration over time in monkey after either IV (left) or PO dosing (right).

FIG. 9A-FIG. 9D shows that β-arrestin-2 (Arrb2) regulates NMDA receptor activity in spinal cord dorsal horn (SCDH) neurons and neuropathic pain. FIG. 9A shows patch-clamp recordings of NMDA-induced inward currents in lamina II neurons of spinal cord slices and the effects of NR2A and NR2B antagonists in WT and Arrb2-KO mice while FIG. 9 B is quantification of NMDA current amplitude. FIG. 9C shows that intrathecal NMDA (1 nmol)-induced spontaneous pain in WT and KO mice. FIG. 9D shows that intra-spinal injection of Arrb2-LV, at 7 days or 112 days, delayed nerve injury (SNL)-induced mechanical allodynia and reversed neuropathic pain in the late-phase (>3 months). In FIG. 9B-FIG. 9D. *p<0.05 as determined by a two-way or one-way ANOVA.

FIG. 10A-FIG. 10E show the effects of SBI-553 on pain, motor function, and spinal cord synaptic transmission. FIG. 10A is a von Frey test showing the effect of intrathecal injection of SBI-553 and vehicle on paclitaxel (PTX)-induced mechanical allodynia. FIG. 10B is rotarod test showing motor function. Each data point is one mouse (n=5 male mice per group, two-way ANOVA, followed by Bonferroni post-hoc test: NS=not significant). FIG. 10C-FIG. 10E are directed to patch-clamp recordings showing the effects of SBI-553 and morphine on sEPSCs in lamina IIo neurons of spinal cord slices prepared from PTX-treated mice. FIG. 10C shows traces of sEPSCs while FIG. 10D shows sEPSC frequency before and after SBI-553 perfusion. FIG. 10E shows the comparison of SBI-553's and morphine's effects on sEPSC frequency (n=5-10 neurons per group, paired Student's t-test).

FIG. 11 shows the effects of SBI-553 (12 mg/kg. IP) on plantar incision-induced post-operative pain (mechanical allodynia) in WT and Arrb2 KO mice (n=4-5 mice, p=0.031, two-way ANOVA).

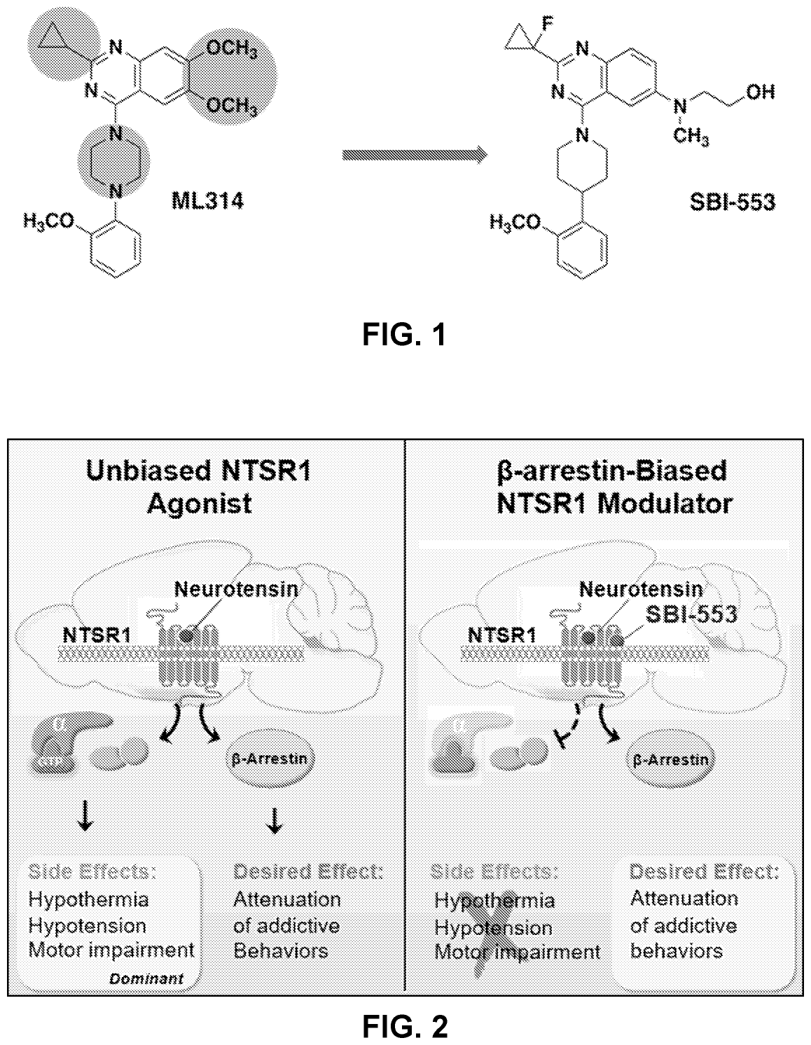

### BRIEF SUMMARY

Disclosed herein is a composition comprising a therapeutically effective amount of a neurotensin receptor 1 (NTSR1) ligand or pharmaceutically acceptable salt, solvate, prodrug, or N-oxide thereof. Disclosed herein is a composition comprising a therapeutically effective amount of a neurotensin receptor 1 (NTSR1) ligand or pharmaceutically acceptable salt, solvate, prodrug, or N-oxide thereof, wherein the composition can treat and/or prevent pain in a subject. Disclosed herein is a composition comprising a therapeutically effective amount of a neurotensin receptor 1 (NTSR1) ligand or pharmaceutically acceptable salt, solvate, prodrug, or N-oxide thereof, wherein the composition can cause the subject to experience less pain, less intense pain, a shorter duration of pain, or a combination thereof. Disclosed herein is a composition comprising a therapeutically effective amount of a neurotensin receptor 1 (NTSR1) ligand or pharmaceutically acceptable salt, solvate, prodrug, or N-oxide thereof, wherein the composition can improve the subject's ability to move and/or level of mobility. Disclosed herein is a composition comprising a therapeutically effective amount of a neurotensin receptor 1 (NTSR1) ligand or pharmaceutically acceptable salt, solvate, prodrug, or N-oxide thereof, wherein the composition does not elicit hypothermia, hypotension, and/or motor impairment in a subject.

Disclosed herein is a composition comprising a therapeutically effective amount of SBI-553. Disclosed herein is a composition comprising a therapeutically effective amount of SBI-553, wherein the composition can treat and/or prevent pain in a subject. Disclosed herein is a composition comprising a therapeutically effective amount of SBI-553, wherein the composition does not elicit hypothermia, hypotension, and/or motor impairment in a subject.

Disclosed herein is a composition comprising a therapeutically effective amount of SBI-810. Disclosed herein is a composition comprising a therapeutically effective amount of SBI-810, wherein the composition can treat and/or prevent pain in a subject. Disclosed herein is a composition comprising a therapeutically effective amount of SBI-810, wherein the composition does not elicit hypothermia, hypotension, and/or motor impairment in a subject.

Disclosed herein is a method of making a disclosed neurotensin receptor 1 (NTSR1) ligand or pharmaceutically acceptable salt, solvate, prodrug, or N-oxide thereof. In an aspect, methods of making and identifying a disclosed NTSR1 ligand or pharmaceutically acceptable salt, solvate, prodrug, or N-oxide thereof are provided by WO 2015/200534, which is incorporated by reference in its entirety for teaching the manufacturing of NTSR1 ligands including but not limited SBI-553 and SBI-810.

Disclosed herein is a pharmaceutical formulation comprising a therapeutically effective amount of a neurotensin receptor 1 (NTSR1) ligand or pharmaceutically acceptable salt, solvate, prodrug, or N-oxide thereof, and a disclosed and a pharmaceutically acceptable carrier.

Disclosed herein is a pharmaceutical formulation comprising a therapeutically effective amount of SBI-553. Disclosed herein is a pharmaceutical formulation comprising a therapeutically effective amount of SBI-553, wherein the pharmaceutical formulation can treat and/or prevent pain in a subject. Disclosed herein is a pharmaceutical formulation comprising a therapeutically effective amount of SBI-553, wherein the pharmaceutical formulation does not elicit hypothermia, hypotension, and/or motor impairment in a subject.

Disclosed herein is a pharmaceutical formulation comprising a therapeutically effective amount of SBI-810. Disclosed herein is a pharmaceutical formulation comprising a therapeutically effective amount of SBI-810, wherein the pharmaceutical formulation can treat and/or prevent pain in a subject. Disclosed herein is a pharmaceutical formulation comprising a therapeutically effective amount of SBI-810, wherein the composition does not elicit hypothermia, hypotension, and/or motor impairment in a subject.

Disclosed herein is a kit comprising a disclosed composition comprising a therapeutically effective amount of a NTSR1 ligand, a disclosed pharmaceutical formulation comprising a therapeutically effective amount of a NTSR1 ligand, or any combination thereof.

Disclosed herein is a kit comprising a disclosed composition comprising a therapeutically effective amount of SBI-553, a disclosed pharmaceutical formulation comprising a therapeutically effective amount of SBI-553, or any combination thereof.

Disclosed herein is a kit comprising a disclosed composition comprising a therapeutically effective amount of SBI-810, a disclosed pharmaceutical formulation comprising a therapeutically effective amount of SBI-810, or any combination thereof.

Disclosed herein is a kit comprising a disclosed composition comprising a therapeutically effective amount of a NTSR1 ligand, a disclosed pharmaceutical formulation comprising a therapeutically effective amount of a NTSR1 ligand, or any combination thereof, and one or more therapeutic agents.

Disclosed herein is a kit comprising a disclosed composition comprising a therapeutically effective amount of SBI-553, a disclosed pharmaceutical formulation comprising a therapeutically effective amount of SBI-553, or any combination thereof, and one or more therapeutic agents.

Disclosed herein is a kit comprising a disclosed composition comprising a therapeutically effective amount of SBI-810, a disclosed pharmaceutical formulation comprising a therapeutically effective amount of SBI-810, or any combination thereof, and one or more therapeutic agents.

Disclosed herein is a method of treating and/or preventing pain in a subject, the method comprising administering to a subject in need thereof a therapeutically effective amount of a disclosed composition or a disclosed pharmaceutical formulation, wherein pain is alleviated and/or minimized in the subject.

Disclosed herein is a method of treating and/or preventing pain in a subject, the method comprising administering to a subject in need thereof a composition or a pharmaceutical formulation comprising a therapeutically effective amount of a NTSR1 ligand or a pharmaceutically acceptable salt, solvate, prodrug, or N-oxide thereof, wherein pain is alleviated and/or minimized in the subject.

Disclosed herein is a method of treating and/or preventing pain in a subject, the method comprising administering to a subject in need thereof a composition comprising a therapeutically effective amount of SBI-553, wherein pain is alleviated and/or minimized in the subject.

Disclosed herein is a method of treating and/or preventing pain in a subject, the method comprising administering to a subject in need thereof a pharmaceutical formulation comprising a therapeutically effective amount of SBI-553, wherein pain is alleviated and/or minimized in the subject.

Disclosed herein is a method of treating and/or preventing pain in a subject, the method comprising administering to a subject in need thereof a composition comprising a therapeutically effective amount of SBI-810, wherein pain is alleviated and/or minimized in the subject.

Disclosed herein is a method of treating and/or preventing pain in a subject, the method comprising administering to a subject in need thereof a pharmaceutical formulation comprising a therapeutically effective amount of SBI-810, wherein pain is alleviated and/or minimized in the subject.

### DETAILED DESCRIPTION

The present disclosure describes formulations, compounded compositions, kits, capsules, containers, and/or methods thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. Neurotensin

Neurotensin is a 13 amino acid neuropeptide implicated in the regulation of luteinizing hormone and prolactin release and has significant interaction with the dopaminergic system. Neurotensin was first isolated from extracts of bovine hypothalamus based on its ability to cause a visible vasodilation in the exposed cutaneous regions of anesthetized rats. Neurotensin is distributed throughout the central nervous system, with highest levels in the hypothalamus, amygdala and nucleus accumbens. It induces a variety of effects, including analgesia, hypothermia and increased locomotor activity. It is also involved in regulation of dopamine pathways. Neurotensin is an endogenous neuropeptide involved in thermoregulation that can induce hypothermia and neuroprotection in experimental models of cerebral ischemia. Outside the CNS, neurotensin is found in endocrine cells of the small intestine, where it leads to secretion and smooth muscle contraction.

### B. Neurotensin Receptors

The neurotensin receptors are transmembrane receptors that bind the neurotransmitter neurotensin. Two of the receptors encoded by the NTSR1 and NTSR2 genes contain seven transmembrane helices and are G protein coupled. The third receptor has a single transmembrane domain and is encoded by the SORT1 gene.

Neurotensin (NT) receptors are expressed on dopaminergic neurological pathways associated with reward, and the neurotensin receptor 1 (NTSR1) is a therapeutic target for the treatment of methamphetamine abuse. In particular, peptide based NTSR1 agonists produce behaviors that are opposite to the psychostimulant effects observed with psychoactive drugs, such as but not limited to methamphetamine, such as hyperactivity, neurotoxicity, psychotic episodes, and cognitive deficits.

NTSR1 is a G protein coupled receptor (GPCR). Two distinct, interdependent paradigms are associated with GPCR signaling. In addition to the well-defined signaling cascades involving heterotrimeric G proteins, recent advances in receptor pharmacology have identified the importance of β-arrestins in regulating alternative biochemical cascades that produce their own unique biological effects. For example, in a mouse model, a series of β-arrestin-2 biased agonists for the D(2)R with antipsychotic properties, and most importantly, a reduced propensity to induce catalepsy like standard neuroleptic antagonists (Allen J A, et al. (2011) Proc Natl Acad Sci USA. 108:18488-18493; Rajagopal S, et al. (2010) Nat. Rev. Drug Discovery. 9:373-386). Studies with those biased compounds illustrate how ligand directed signaling bias, in this case favoring β-arrestin, can ameliorate undesirable biological outcomes. Downstream modulators of β-arrestin/GPCR signaling are less characterized than their G protein counterparts, and, due to their potential as targets for producing new medical therapies are the subjects of increasing numbers of investigations. Recognized β-arrestin partners include the proteins Src, ERK, and Jnk. Their agonist-induced interactions with β-arrestin are associated with clathrin-compartmentalized signaling and the accumulation of ligand activated β-arrestin/GPCR complexes in clathrin coated pits. The determination as to whether a GPCR ligand is biased towards or against β-arrestin may consequently be evaluated by following these biochemical processes.

### C. Definitions

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method. The phrase “consisting of” excludes any component, step, or element that is not recited in the claim. The phrase “comprising” is synonymous with “including”, “containing”, or “characterized by”, and is inclusive or open-ended. “Comprising” does not exclude additional, unrecited components or steps.

As used herein, when referring to any numerical value, the term “about” means a value falling within a range that is ±10% of the stated value.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

As used herein, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

As used herein, the term “subject” refers to the target of administration, e.g., a human being. In an aspect, a subject can be in the military or can be a veteran. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, geriatric, adult, adolescent, and child subjects, as well as fetuses, whether male or female, are intended to be covered. In an aspect, a subject can be a human subject. In an aspect, a subject can have pain, be suspected of having pain, or be at risk of developing pain. In an aspect, a subject's pain can be due to one or more bone fractures. In an aspect, a subject's pain can be due to something other bone fractures or injuries. In an aspect, a subject's pain can be due to cancer or a chronic disease. In an aspect, a subject's pain can be neuropathic pain. In an aspect, a subject's pain can be due to genetic disease and/or genetic disorder. In an aspect, a subject's acute and/or chronic pain can comprise back pain (including upper back pain and/or lower back pain), cancer pain, arthritis pain, headaches (including but not limited to migraines, cluster headaches, and tension headaches), neuralgias, neuropathies, pain due to a CNS injury, pain due to a PNS injury, pain with no known etiological cause, or any combination thereof.

As used herein, the term “diagnosed” means having been subjected to an examination by a person of skill, for example, a physician, and found to have a condition that can be diagnosed or treated by one or more of the disclosed compositions, disclosed pharmaceutical formulations, or a combination thereof, or by one or more of the disclosed methods. For example, “diagnosed with pain” or “diagnosed with a need for non-opioid analgesia” means having been subjected to an examination by a person of skill, for example, a physician, and found to have a condition that can be treated by one or more of the disclosed compositions, disclosed pharmaceutical formulations, or a combination thereof, or by one or more of the disclosed methods. For example, “suspected of having pain” can mean having been subjected to an examination by a person of skill, for example, a physician, and found to have a condition that can likely be treated by one or more of the disclosed compositions, disclosed pharmaceutical formulations, or a combination thereof, or by one or more of the disclosed methods. In an aspect, an examination can be physical, can involve various tests (e.g., blood tests, genotyping, biopsies, etc.), diagnostic evaluations (e.g., X-ray, CT scan, etc.), and assays (e.g., enzymatic assay), or a combination thereof. In an aspect, an examination can be objective and/or subjective.

A “patient” refers can refer to a subject afflicted with a disease or disorder (e.g., acute pain and/or chronic pain). In an aspect, a patient can be in the military or can be a veteran. In an aspect, a patient can refer to a subject that has been diagnosed with or is suspected of having a disease or disorder such as acute pain and/or chronic pain. In an aspect, a patient can refer to a subject that has been diagnosed with or is suspected of having a disease or disorder and is seeking treatment or receiving treatment for a disease or disorder (such as acute pain and/or chronic pain). In an aspect, a “patient” can refer to a subject afflicted with acute pain and/or chronic pain. In an aspect, a patient can refer to a subject that has been diagnosed with or is suspected of having a disease or disorder such as acute pain and/or chronic pain. In an aspect, a patient can refer to a subject that has been diagnosed with or is suspected of having a disease or disorder and is seeking treatment or receiving treatment for a disease or disorder (such as acute pain and/or chronic pain).

In an aspect, while acute pain is a normal sensation triggered in the nervous system to alert an individual to possible injury and the need to take care of the situation or issue, chronic pain is different. Chronic pain persists. Pain signals keep firing in the nervous system for weeks, months, even years. There may have been an initial incident and/or mishap such as a sprained back and/or serious infection, or there may be an ongoing cause of pain such as cancer and/or ear infection, but some people suffer chronic pain in the absence of any past injury or evidence of body damage. Many chronic pain conditions affect older adults. Common chronic pain complaints include headache, low back pain, cancer pain, arthritis pain, neurogenic pain (pain resulting from damage to the peripheral nerves or to the central nervous system itself), psychogenic pain (pain not due to past disease or injury or any visible sign of damage inside or outside the nervous system).

In an aspect, a disclosed disease or disorder can comprise an acute stress disorder, alcohol abuse, alcohol dependence, alcohol withdrawal, alcoholic hallucinosis, Alzheimer's disease, amphetamine dependence, amphetamine withdrawal psychosis, anorexia nervosa, anxiety disorder, anxiolytic-related disorders, Asperger syndrome, attention deficit disorder, attention deficit hyperactivity disorder, autism, barbiturate dependence, benzodiazepine dependence, benzodiazepine misuse, benzodiazepine withdrawal, bipolar disorder, bipolar I disorder, bipolar II disorder, bulimia nervosa, cannabis dependence, catatonic disorder, catatonic schizophrenia, cocaine dependence, cocaine intoxication, cotard delusion, cyclothymia, delirium tremens, depressive disorder, generalized anxiety disorder, grandiose delusions, hallucinogen-related disorder, hallucinogen persisting perception disorder, Huntington's disease, impulse control disorder, intermittent explosive disorder, major depressive disorder, major depressive episode, manic episode, minor depressive disorder, minor depressive episode, Munchhausen's syndrome, neuroleptic-related disorder, night eating syndrome, obsessive-compulsive disorder (OCD), opioid dependence, opioid withdrawal, opioid overdose, pain disorder, panic disorder, paranoid personality disorder, parasomnia. Parkinson's disease, partner relational problem, pathological gambling, phencyclidine (or phencyclidine-like)-related disorder, residual schizophrenia, sadomasochism, schizoaffective disorder, schizoid personality disorder, schizophrenia, schizophreniform disorder, schizotypal personality disorder, social anxiety disorder, social phobia, substance-related disorder, tardive dyskinesia, Tourette syndrome, or ischemia-induced cognitive deficits.

As used herein, the phrase “identified to be in need of treatment for pain,” or the like, refers to selection of a subject based upon need for treatment of acute and/or chronic pain. For example, a subject can be identified as having a need for treatment of pain (e.g., due to acute and/or chronic pain) based upon an earlier diagnosis by a person of skill and thereafter subjected to treatment for pain (e.g., due to acute and/or chronic pain). In an aspect, the identification can be performed by a person different from the person making the diagnosis. In an aspect, the administration can be performed by one who performed the diagnosis.

As used herein, “inhibit,” “inhibiting”, and “inhibition” mean to diminish or decrease an activity, level, response, condition, severity, disease, or other biological parameter. In an aspect, “inhibiting” can refer to diminishing the intensity, the duration, the amount, or a combination thereof of a subject's pain (e.g., acute and/or chronic pain due to trauma, injury, cancer, disease and/or disorder, neuropathic pain, etc.). This can include, but is not limited to, the complete ablation of the activity, level, response, condition, severity, disease, or other biological parameter. This can also include, for example, a 10% inhibition or reduction in the activity, level, response, condition, severity, disease, or other biological parameter as compared to the native or control level (e.g., a subject not having pain) or to the subject's pain level prior to the onset of pain (e.g., acute and/or chronic pain due to trauma, injury, cancer, disease and/or disorder, neuropathic pain. etc.). Thus, in an aspect, the inhibition or reduction can be a 10%, 20%, 30%, 40%, 50%, 60%, 70%, 80%, 90%, 100%, or any amount of reduction in between as compared to native or control levels or to the subject's level prior to prior to the onset of pain (e.g., acute and/or chronic pain due to trauma, injury, cancer, disease and/or disorder, neuropathic pain, etc.). In an aspect, the inhibition or reduction can be 10-20%, 20-30%, 30-40%, 40-50%, 50-60%, 60-70%, 70-80%, 80-90%, or 90-100% as compared to native or control levels or to the subject's level prior to the onset of pain (e.g., acute and/or chronic pain due to trauma, injury, cancer, disease and/or disorder, neuropathic pain, etc.). In an aspect, the inhibition or reduction can be 0-25%, 25-50%, 50-75%, or 75-100% as compared to native or control levels or to the subject's pain level prior to the onset of pain (e.g., acute and/or chronic pain due to trauma, injury, cancer, disease and/or disorder, neuropathic pain, etc.). In an aspect, the subject's pain level prior to the onset of pain can be used as a control.

In an aspect, a subject's acute and/or chronic pain can comprise back pain (including upper back pain and/or lower back pain), cancer pain, arthritis pain, headaches (including but not limited to migraines, cluster headaches, and tension headaches), neuralgias, neuropathies, pain due to a CNS injury, pain due to a PNS injury, pain with no known etiological cause, or any combination thereof. In an aspect, a subject's pain can be cancer pain. In an aspect, a subject's pain can be arthritis pain. In an aspect, a subject's pain can be headache pain. In an aspect, a subject's pain can be neuralgia pain. In an aspect, a subject's pain can be neuropathy pain. In an aspect, a subject's pain can be due to an injured sustained while serving in the armed forces or military.

The words “treat” or “treating” or “treatment” include palliative treatment, that is, treatment designed for the relief of symptoms rather than the curing of the acute and/or chronic pain, disease, pathological condition, or disorder; preventative treatment, that is, treatment directed to minimizing or partially or completely inhibiting the development of the of the acute and/or chronic pain, associated disease, pathological condition, or disorder; and supportive treatment, that is, treatment employed to supplement another specific therapy directed toward the improvement of the associated of the acute and/or chronic pain, disease, pathological condition, or disorder. In an aspect, the terms cover any treatment of a subject, including a mammal (e.g., a human), and includes: (i) preventing the undesired physiological change, acute and/or chronic pain, disease, pathological condition, or disorder from occurring in a subject that can be predisposed to the disease but has not yet been diagnosed as having it; (ii) inhibiting the physiological change, acute and/or chronic pain, disease, pathological condition, or disorder, i.e., arresting its development; or (iii) relieving the physiological change, acute and/or chronic pain, disease, pathological condition, or disorder, i.e., causing regression of the disease. For example, in an aspect, treating a disease or disorder can reduce the severity of an established a disease or disorder in a subject by 1%-100% as compared to a control (such as, for example, an individual not having acute pain and/or chronic pain). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of a disease or a disorder or a condition (such as acute pain and/or chronic pain). For example, treating a disease or a disorder can reduce one or more symptoms of a disease or disorder in a subject by 1%-100% as compared to a control (such as, for example, an individual not having acute pain and/or chronic pain). In an aspect, treating can refer to 1%, 2%, 3%, 4%, 5%, 6%, 7%, 8%, 9%, 10%, 20%, 30%, 40%, 50%, 60%, 70%, 80%, 90%, 100% reduction of one or more symptoms of an established a disease or a disorder or a condition (e.g., acute pain and/or chronic pain). It is understood that treatment does not necessarily refer to a cure or complete ablation or eradication of a disease or disorder or acute pain and/or chronic pain. However, in an aspect, treatment can refer to a cure or complete ablation or eradication of a disease or a disorder or a condition (such as acute pain and/or chronic pain).

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing pain or the worsening of pain is intended. The words “prevent” and “preventing” and “prevention” also refer to prophylactic or preventative measures for protecting or precluding a subject (e.g., an individual) not having pain or a given pain-related complication from progressing to that complication (such as, for example, acute, chronic, and/or debilitating pain).

As used herein, the terms “administering” and “administration” refer to any method of providing one or more of the disclosed compositions, disclosed pharmaceutical formulations, or a combination thereof. Such methods are well-known to those skilled in the art and include, but are not limited to, the following: oral administration, transdermal administration, administration by inhalation, nasal administration, topical administration, in utero administration, intrahepatic administration, intravaginal administration, epidural administration (such as epidural injection), intracerebroventricular (ICV) administration, ophthalmic administration, intraaural administration, depot administration, topical (skin) administration, otic administration, intra-articular (such as joint or vertebrate injection), intracerebral administration, rectal administration, sublingual administration, buccal administration, and parenteral administration, including injectable such as intravenous administration, intra-CSF administration, intra-cistern magna (ICM) administration, intra-arterial administration, intrathecal (ITH) administration, intramuscular administration, and subcutaneous administration. Administration can also include hepatic intra-arterial administration or administration through the hepatic portal vein (HPV). Administration of a disclosed composition, a disclosed pharmaceutical formulation, a disclosed therapeutic agent, a disclosed immune modulator, a disclosed proteasome inhibitor, a disclosed small molecule, a disclosed endonuclease, a disclosed oligonucleotide, and/or a disclosed RNA therapeutic can comprise administration directly into the CNS or the PNS. Administration can be continuous or intermittent. Administration can comprise a combination of one or more route. In an aspect, a disclosed composition, a disclosed pharmaceutical formulation, or any combination thereof can be concurrently and/or serially administered to a subject via multiple routes of administration. For example, in an aspect, administering a disclosed composition, a disclosed pharmaceutical formulation, or any combination thereof can comprise intravenous administration and intra-cistern magna (ICM) administration. In an aspect, administering a disclosed composition, a disclosed pharmaceutical formulation, or any combination thereof can comprise IV administration and intrathecal (ITH) administration. Various combinations of administration are known to the skilled person.

In an aspect, the skilled person can determine an efficacious dose, an efficacious schedule, and an efficacious route of administration for the disclosed compositions, disclosed pharmaceutical formulations, or a combination thereof to treat or prevent a disease or disorder (such as acute pain and/or chronic pain). In an aspect, the skilled person can also alter, change. or modify an aspect of an administering step to improve efficacy of the disclosed compositions, disclosed pharmaceutical formulations, or a combination thereof.

By “determining the amount” is meant both an absolute quantification of a particular analyte (e.g., biomarker for acute pain and/or chronic pain) or a determination of the relative abundance of a particular analyte (e.g., a biomarker). The phrase includes both direct or indirect measurements of abundance or both.

As used herein, “modifying the method” can comprise modifying or changing one or more features or aspects of one or more steps of a disclosed method. In an aspect, a method can be altered by changing the amount of the disclosed compositions, disclosed pharmaceutical formulations, disclosed therapeutic agents, or a combination thereof administered to a subject, or by changing the frequency of administration of the disclosed compositions, disclosed pharmaceutical formulations, disclosed therapeutic agents, or a combination thereof to a subject, by changing the duration of time that the disclosed compositions, disclosed pharmaceutical formulations, disclosed therapeutic agents, or a combination thereof is administered to a subject, or by substituting for one or more of the disclosed components and/or reagents with a similar or equivalent component and/or reagent. The same applies to all disclosed compositions, disclosed pharmaceutical formulations, disclosed therapeutic agents, or a combination thereof.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. In an aspect, a pharmaceutical carrier employed can be a solid, liquid, or gas. In an aspect, examples of solid carriers can include lactose, terra alba, sucrose, talc, gelatin, agar, pectin, acacia, magnesium stearate, and stearic acid. In an aspect, examples of liquid carriers can include sugar syrup, peanut oil, olive oil, and water. In an aspect, examples of gaseous carriers can include carbon dioxide and nitrogen. In preparing a disclosed composition for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions: while carriers such as starches. sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents. such as aluminum monostearate and gelatin, which delay absorption. Injectable depot forms are made by forming microencapsule matrices of the drug in biodegradable polymers such as polylactide-polyglycolide, poly (orthoesters) and poly (anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “excipient” refers to an inert substance which is commonly used as a diluent, vehicle, preservative, binder, or stabilizing agent, and includes, but is not limited to. proteins (e.g., serum albumin, etc.), amino acids (e.g., aspartic acid, glutamic acid, lysine. arginine, glycine, histidine, etc.), fatty acids and phospholipids (e.g., alkyl sulfonates, caprylate. etc.), surfactants (e.g., SDS, polysorbate, nonionic surfactant, etc.), saccharides (e.g., sucrose. maltose, trehalose, etc.) and polyols (e.g., mannitol, sorbitol, etc.). See, also, for reference. Remington's Pharmaceutical Sciences. (1990) Mack Publishing Co., Easton. Pa., which is hereby incorporated by reference in its entirety for the teaching of excipients.

As used herein. “concurrently” means (1) simultaneously in time, or (2) at different times during a common treatment schedule.

The term “contacting” as used herein refers to bringing one or more of the disclosed compositions, disclosed pharmaceutical formulations, disclosed therapeutic agents, or a combination thereof together with a target area or intended target area in such a manner that the disclosed compositions, disclosed pharmaceutical formulations, disclosed therapeutic agents, or a combination thereof can exert an effect on the intended target or targeted area either directly or indirectly. A target area or intended target area can be one or more of a subject's organs or a subject's limbs or any source of acute pain and/or chronic pain. In an aspect, a target area or intended target area can be any cell or any organ infected by a disease or disorder (such as, for example, cancer). In an aspect, a target area or intended target area can be any organ, tissue, or cells that are affected by a disease or disorder such that the subject has acute pain and/or chronic pain).

As used herein, “determining” can refer to measuring or ascertaining the presence and severity of a disease or disorder, such as, for example, acute and/or chronic pain. “Determining” can refer to measuring or ascertaining the presence or severing or a subject's acute pain and/or chronic pain. Methods and techniques used to determine the presence and/or severity of a disease or disorder are typically known to the medical arts. For example, the art is familiar with the ways to identify and/or diagnose the presence, severity, or both of acute pain and/or chronic pain. Methods can be based on objective and/or subjective means.

As used herein, “effective amount” and “amount effective” can refer to an amount that is sufficient to achieve the desired result such as, for example, the treatment and/or prevention of acute pain and/or chronic pain. As used herein, the terms “effective amount” and “amount effective” can refer to an amount that is sufficient to achieve the desired an effect on an undesired condition (e.g., acute pain and/or chronic pain). For example, a “therapeutically effective amount” refers to an amount that is sufficient to achieve the desired therapeutic result or to have an effect on undesired symptoms, but is generally insufficient to cause adverse side effects. In an aspect, “therapeutically effective amount” means an amount of the disclosed compositions, disclosed pharmaceutical formulations, or a combination thereof: that (i) treats the acute pain and/or chronic pain associated with a disease, condition, or disorder (e.g., back injury, cancer, neuropathy, neuralgia, etc.), (ii) attenuates, ameliorates, or eliminates one or more symptoms of acute pain and/or chronic pain associated with a particular disease, condition, or disorder (e.g., back injury, cancer, neuropathy, neuralgia, etc.), or (iii) delays the onset of one or more symptoms of acute pain and/or chronic pain (e.g., back injury, cancer, neuropathy, neuralgia, etc.). The specific therapeutically effective dose level for any particular patient will depend upon a variety of factors including the disorder being treated and the severity of the acute pain and/or chronic pain; the disclosed compositions, disclosed pharmaceutical formulations, disclosed therapeutic agents, or a combination thereof employed; the disclosed methods employed: the age, body weight, general health, sex and diet of the patient; the time of administration: the route of administration; the rate of excretion of the disclosed compositions, disclosed pharmaceutical formulation, disclosed therapeutic agents, or a combination thereof employed; the duration of the treatment; drugs used in combination or coincidental with the disclosed compositions, disclosed pharmaceutical formulations, disclosed therapeutic agents, or a combination thereof employed, and other like factors well known in the medical arts. For example, it is well within the skill of the art to start doses of the disclosed interfering molecules, the disclosed compositions, disclosed pharmaceutical formulations, disclosed therapeutic agents, or a combination thereof at levels lower than those required to achieve the desired therapeutic effect and to gradually increase the dosage until the desired effect (e.g., less or no pain) is achieved. If desired, then the effective daily dose can be divided into multiple doses for purposes of administration. Consequently, a single dose of the disclosed compositions, disclosed pharmaceutical formulations, disclosed therapeutic agents, or a combination thereof can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention if acute pain and/or chronic pain, such as, for example, acute pain and/or chronic pain associated with a disease or disorder.

As used herein, “immune-modulating” refers to the ability of a disclosed therapeutic agent to alter (modulate) one or more aspects of the immune system. The immune system functions to protect the organism from infection and from foreign antigens by cellular and humoral mechanisms involving lymphocytes, macrophages, and other antigen-presenting cells that regulate each other by means of multiple cell-cell interactions and by elaborating soluble factors. including lymphokines and antibodies, that have autocrine, paracrine, and endocrine effects on immune cells.

As used herein, “immune modulator” refers to an agent that is capable of adjusting a given immune response to a desired level (e.g., as in immunopotentiation, immunosuppression, or induction of immunologic tolerance). Examples of immune modulators include but are not limited to, a disclosed immune modulator can comprise aspirin, azathioprine, belimumab, betamethasone dipropionate, betamethasone valerate, bortezomib, bredinin, cyazathioprine, cyclophosphamide, cyclosporine, deoxyspergualin, didemnin B, fluocinolone acetonide, folinic acid, ibuprofen, IL6 inhibitors (such as sarilumab) indomethacin, inebilizumab, intravenous gamma globulin (IVIG), methotrexate, methylprednisolone, mycophenolate mofetil, naproxen, prednisolone, prednisone, prednisolone indomethacin, rapamycin, rituximab, sirolimus, sulindac, synthetic vaccine particles containing rapamycin (SVP-Rapamycin or ImmTOR), thalidomide, tocilizumab, tolmetin, triamcinolone acetonide, anti-CD3 antibodies, anti-CD4 antibodies, anti-CD19 antibodies, anti-CD20) antibodies, anti-CD22 antibodies, anti-CD40) antibodies, anti-FcRN antibodies, anti-IL6 antibodies, anti-IGF1R antibodies, an IL2 mutein, a BTK inhibitor, or a combination thereof. In an aspect, a disclosed immune modulator can comprise one or more Treg (regulatory T cells) infusions (e.g., antigen specific Treg cells to AAV). In an aspect, a disclosed immune modulator can be bortezomib or SVP-Rapamycin. In an aspect, an immune modulator can be administered by any suitable route of administration including, but not limited to, in utero, intra-CSF, intrathecally, intravenously, subcutaneously, transdermally, intradermally, intramuscularly, orally, transcutaneously, intraperitoneally (IP), or intravaginally. In an aspect, a disclosed immune modulator can be administered using a combination of routes. Administration can also include hepatic intra-arterial administration or administration through the hepatic portal vein (HPV). Administration of an immune modulator can be continuous or intermittent, and administration can comprise a combination of one or more routes.

As used herein, the term “package insert” is used to refer to instructions customarily included in commercial packages of therapeutic products, that contain information about the indications, usage, dosage, administration, contraindications and/or warnings concerning the use of such therapeutic products.

As used herein, the term “in combination” in the context of the administration of other therapies (e.g., other agents) includes the use of more than one therapy (e.g., drug therapy). Administration “in combination with” one or more further therapeutic agents includes simultaneous (e.g., concurrent) and consecutive administration in any order. The use of the term “in combination” does not restrict the order in which therapies are administered to a subject. By way of non-limiting example, a first therapy (e.g., a disclosed composition, a disclosed pharmaceutical formulation, or a combination thereof) may be administered prior to (e.g., 1 minute, 15 minutes, 30) minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy (e.g., a disclosed composition, a disclosed pharmaceutical formulation, a disclosed therapeutic agent, or a combination thereof) to a subject having or diagnosed with cancer.

Disclosed are the components to be used to prepare the disclosed compositions, disclosed pharmaceutical formulations, disclosed therapeutic agents, or a combination thereof used within the methods disclosed herein. These and other materials are disclosed herein, and it is understood that when combinations, subsets, interactions, groups, etc, of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

### 1. Therapeutic Agents

a. Biologically Active Agents

As used herein, the term “biologically active agent” or “biologic active agent” or “bioactive agent” means an agent that is capable of providing a local or systemic biological, physiological, or therapeutic effect in the biological system to which it is applied. For example, the bioactive agent can act to control infection or inflammation, enhance cell growth and tissue regeneration, control tumor growth, act as an analgesic, promote anti-cell attachment, and enhance bone growth, among other functions. Other suitable bioactive agents can include anti-viral agents, vaccines, hormones, antibodies (including active antibody fragments sFv, Fv, and Fab fragments), aptamers, peptide mimetics, functional nucleic acids, therapeutic proteins, peptides, or nucleic acids. Other bioactive agents include prodrugs, which are agents that are not biologically active when administered but, upon administration to a subject are converted to bioactive agents through metabolism or some other mechanism. Additionally, any of the compositions of the invention can contain combinations of two or more bioactive agents. It is understood that a biologically active agent can be used in connection with administration to various subjects, for example, to humans (i.e., medical administration) or to animals (i.e., veterinary administration). As used herein, the recitation of a biologically active agent inherently encompasses the pharmaceutically acceptable salts thereof.

b. Pharmaceutically Active Agents

As used herein, the term “pharmaceutically active agent” includes a “drug” or a “vaccine” and means a molecule, group of molecules, complex or substance administered to an organism for diagnostic, therapeutic, preventative medical, or veterinary purposes. This term includes externally and internally administered topical, localized and systemic human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term may also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. This term can also specifically include nucleic acids and compounds comprising nucleic acids that produce a bioactive effect, for example deoxyribonucleic acid (DNA) or ribonucleic acid (RNA). Pharmaceutically active agents include the herein disclosed categories and specific examples. It is not intended that the category be limited by the specific examples. Those of ordinary skill in the art will recognize also numerous other compounds that fall within the categories and that are useful according to the invention. Examples include a radiosensitizer, the combination of a radiosensitizer and a chemotherapeutic, a steroid, a xanthine, a beta-2-agonist bronchodilator, an anti-inflammatory agent, an analgesic agent, a calcium antagonist, an angiotensin-converting enzyme inhibitors, a beta-blocker, a centrally active alpha-agonist, an alpha-1-antagonist, carbonic anhydrase inhibitors, prostaglandin analogs, a combination of an alpha agonist and a beta blocker, a combination of a carbonic anhydrase inhibitor and a beta blocker, an anticholinergic/antispasmodic agent, a vasopressin analogue, an antiarrhythmic agent, an antiparkinsonian agent, an antiangina/antihypertensive agent, an anticoagulant agent, an antiplatelet agent, a sedative, an ansiolytic agent, a peptidic agent, a biopolymeric agent, an antineoplastic agent, a laxative, an antidiarrheal agent, an antimicrobial agent, an antifungal agent, or a vaccine. In a further aspect, the pharmaceutically active agent can be coumarin, albumin, bromolidine, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenterol, clenbuterol, bambuterol, salmeterol, fenoterol; antiinflammatory agents, including antiasthmatic anti-inflammatory agents, antiarthritis antiinflammatory agents, and non-steroidal antiinflammatory agents, examples of which include but are not limited to sulfides, mesalamine, budesonide, salazopyrin, diclofenac, pharmaceutically acceptable diclofenac salts, nimesulide, naproxene, acetominophen, ibuprofen, ketoprofen and piroxicam; analgesic agents such as salicylates; calcium channel blockers such as nifedipine, amlodipine, and nicardipine; angiotensin-converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, timol hemihydrate, levobunolol hydrochloride, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists (i.e., alpha adrenergic receptor agonist) such as clonidine, brimonidine tartrate, and apraclonidine hydrochloride; alpha-1-antagonists such as doxazosin and prazosin; anticholinergic/antispasmodic agents such as dicyclomine hydrochloride, scopolamine hydrobromide, glycopyrrolate, clidinium bromide, flavoxate, and oxy butynin; vasopressin analogues such as vasopressin and desmopressin; prostaglandin analogs such as latanoprost, travoprost, and bimatoprost; cholinergics (i.e., acetylcholine receptor agonists) such as pilocarpine hydrochloride and carbachol; glutamate receptor agonists such as the N-methyl D-aspartate receptor agonist memantine; anti-Vascular endothelial growth factor (VEGF) aptamers such as pegaptanib; anti-VEGF antibodies (including but not limited to anti-VEGF-A antibodies) such as ranibizumab and bevacizumab; carbonic anhydrase inhibitors such as methazolamide, brinzolamide, dorzolamide hydrochloride, and acetazolamide; antiarrhythmic agents such as quinidine, lidocaine, tocainide hydrochloride, mexiletine hydrochloride, digoxin, verapamil hydrochloride, propafenone hydrochloride, flecaimide acetate, procainamide hydrochloride, moricizine hydrochloride, and diisopyramide phosphate; antiparkinsonian agents, such as dopamine, L-Dopa/Carbidopa, selegiline, dihydroergocryptine, pergolide, lisuride, apomorphine, and bromocryptine; antiangina agents and antihypertensive agents such as isosorbide mononitrate, isosorbide dinitrate, propranolol, atenolol and verapamil; anticoagulant and antiplatelet agents such as coumadin, warfarin, acetylsalicylic acid, and ticlopidine; sedatives such as benzodiazepines and barbiturates; ansiolytic agents such as lorazepam, bromazepam, and diazepam: peptidic and biopolymeric agents such as calcitonin, leuprolide and other LHRH agonists, hirudin, cyclosporin, insulin, somatostatin, protirelin, interferon, desmopressin. somatotropin, thy mopentin, pidotimod, erythropoietin, interleukins, melatonin. granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hydrochloride, and microorganisms; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides. It is understood that a pharmaceutically active agent can be used in connection with administration to various subjects, for example, to humans (i.e., medical administration) or to animals (i.e., veterinary administration). As used herein, the recitation of a pharmaceutically active agent inherently encompasses the pharmaceutically acceptable salts thereof.

c. Chemotherapeutic Agents

As used herein, a “chemotherapeutic agent” can comprise an anthracycline, a vinca alkaloid, an alkylating agent, an immune cell antibody, an antimetabolite, a TNFR glucocorticoid induced TNFR related protein (GITR) agonist, a proteasome inhibitor, an immunomodulator, or any combination thereof. In an aspect, a disclosed chemotherapeutic agent can comprise 5-fluorouracil (Adrucil, Efudex), 6-mercaptopurine (Purinethol), 6-thioguanine, aclarubicin or aclacinomycin A, alemtuzamab (Lemtrada), anastrozole (Arimidex), bicalutamide (Casodex), bleomycin sulfate (Blenoxane), bortezomib (Velcade), busulfan (Myleran), busulfan injection (Busulfex), capecitabine (Xeloda), carboplatin (Paraplatin), carmustine (BiCNU), chlorambucil (Leukeran), cisplatin (Platinol), cladribine (Leustatin), Cosmegan, cyclophosphamide (Cytoxan or Neosar), cyclophosphamide, cytarabine liposome injection (DepoCyt), cytarabine, cytosine arabinoside (Cytosar-U), dacarbazine (DTIC-Dome), dactinomycin (Cosmegen), daunorubicin citrate liposome injection (DaunoXome), daunorubicin hydrochloride (Cerubidine), dexamethasone, docetaxel (Taxotere), doxorubicin hydrochloride (Adriamycin, Rubex), etoposide (Vepesid), fludarabine phosphate (Fludara), flutamide (Eulexin), folic acid antagonists, gemcitabine (difluorodeoxycitidine), gemtuzumab, gliotoxin, hydroxyurea (Hydrea). Idarubicin (Idamycin), ifosfamide (IFEX), ifosfamide, irinotecan (Camptosar), L-asparaginase (ELSPAR), lenalidomide), leucovorin calcium, melphalan (Alkeran), melphalan, methotrexate (Folex), mitoxantrone (Novantrone), mylotarg, N4-pentoxycarbonyl-5 deoxy-5-fluorocytidine, nab-paclitaxel (Abraxane), paclitaxel (Taxol), pentostatin, phoenix (Yttrium90/MX-DTPA), polifeprosan 20 with carmustine implant (Gliadel), purine analogs and adenosine deaminase inhibitors (fludarabine), pyrimidine analogs, rituximab, tamoxifen citrate (Nolvadex), temozolomide), teniposide (Vumon), tezacitibine, thalidomide or a thalidomide derivative, thiotepa, tirapazamine (Tirazone), topotecan hydrochloride for injection (Hycamptin), tositumomab), vinblastine (Velban), vinblastine, vincristine (Oncovin), vindesine, vinorelbine (Navelbine), or any combination thereof.

d. Anti-Bacterial Agents

As used herein, anti-bacterial agents are known to the art. For example, the art generally recognizes several categories of anti-bacterial agents including (1) penicillins, (2) cephalosporins, (3) quinolones, (4) aminoglycosides, (5) monobactams, (6) carbapenems, (7) macrolides, and (8) other agents, For example, as used herein, an anti-bacterial agent can comprise Afenide, Amikacin, Amoxicillin, Ampicillin, Arsphenamine, Augmentin, Azithromycin, Azlocillin, Aztreonam, Bacampicillin, Bacitracin, Balofloxacin, Besifloxacin, Capreomycin, Carbacephem (loracarbef), Carbenicillin, Cefacetrile (cephacetrile), Cefaclomezine, Cefaclor, Cefadroxil (cefadroxyl), Cefalexin (cephalexin), Cefaloglycin (cephaloglycin), Cefalonium (cephalonium), Cefaloram, Cefaloridine (cephaloradine), Cefalotin (cephalothin), Cefamandole, Cefaparole, Cefapirin (cephapirin), Cefatrizine, Cefazaflur, Cefazedone, Cefazolin (cephazolin), Cefcanel, Cefcapene, Cefclidine, Cefdaloxime, Cefdinir, Cefditoren, Cefedrolor, Cefempidone, Cefepime, Cefetamet, Cefetrizole, Cefivitril, Cefixime, Cefluprenam, Cefmatilen, Cefmenoxime, Cefmepidium, Cefmetazole, Cefodizime, Cefonicid, Cefoperazone, Cefoselis, Cefotaxime, Cefotetan, Cefovecin, Cefoxazole, Cefoxitin, Cefozopran, Cefpimizole, Cefpirome, Cefpodoxime, Cefprozil (cefproxil), Cefquinome, Cefradine (cephradine), Cefrotil, Cefroxadine, Cefsumide, Ceftaroline, Ceftazidime, Ceftazidime/Avibactam, Cefteram, Ceftezole, Ceftibuten, Ceftiofur, Ceftiolene, Ceftioxide, Ceftizoxime, Ceftobiprole, Ceftriaxone, Cefuracetime, Cefuroxime, Cefuzonam, Cephalexin, Chloramphenicol, Chlorhexidine, Ciprofloxacin, Clarithromycin, Clavulanic Acid, Clinafloxacin, Clindamycin, Cloxacillin, Colimycin, Colistimethate, Colistin, Crysticillin, Cycloserine 2, Demeclocycline, Dicloxacillin, Dirithromycin, Doripenem, Doxycycline, Efprozil, Enoxacin, Ertapenem, Erythromycin, Ethambutol, Flucloxacillin, Flumequine, Fosfomycin, Furazolidone, Gatifloxacin, Geldanamycin, Gemifloxacin, Gentamicin, Glycopeptides, Grepafloxacin, Herbimycin, Imipenem, Isoniazid, Kanamycin, Levofloxacin, Lincomycin, Linezolid, Lipoglycopeptides, Lomefloxacin, Meropenem, Meticillin, Metronidazole, Mezlocillin, Minocycline, Mitomycin, Moxifloxacin, Mupirocin, Nadifloxacin, Nafcillin, Nalidixic Acid, Neomycin, Netilmicin, Nitrofurantoin, Norfloxacin, Ofloxacin, Oxacillin, Oxazolidinones, Oxolinic Acid, Oxytetracycline, Oxytetracycline, Paromomycin, Pazufloxacin, Pefloxacin, Penicillin G, Penicillin V, Pipemidic Acid, Piperacillin, Piromidic Acid, Pivampicillin, Pivmecillinam, Platensimycin, Polymyxin B, Pristinamycin, Prontosil, Prulifloxacin, Pvampicillin, Pyrazinamide, Quinupristin/dalfopristin, Rifabutin, Rifalazil, Rifampin, Rifamycin, Rifapentine, Rosoxacin, Roxithromycin, Rufloxacin, Sitafloxacin, Sparfloxacin, Spectinomycin, Spiramycin, Streptomycin, Sulbactam, Sulfacetamide, Sulfamethizole, Sulfamethoxazole, Sulfanilimide, Sulfisoxazole, Sulphonamides, Sultamicillin, Teicoplanin, Telavancin, Telithromycin, Temafloxacin, Tetracycline, Thiamphenicol, Ticarcillin, Tigecycline, Tinidazole, Tobramycin, Tosufloxacin, Trimethoprim, Trimethoprim-Sulfamethoxazole, Troleandomycin, Trovafloxacin, Tuberactinomycin, Vancomycin, Viomycin, or pharmaceutically acceptable salts thereof (e.g., such as, for example, chloride, bromide, iodide. and periodate), or a combination thereof. As used herein, the recitation of an anti-bacterial agent inherently encompasses the pharmaceutically acceptable salts thereof.

e. Anti-Fungal Agents

Anti-fungal agents are known to the art. The art generally recognizes several categories of anti-fungal agents including (1) azoles (imidazoles), (2) antimetabolites, (3) allylamines, (4) morpholine, (5) glucan synthesis inhibitors (echinocandins), (6) polyenes, (7) benoxaaborale: (8) other antifungal/onychomycosis agents, and (9) new classes of antifungal/onychomycosis agents. For example, as used herein, an anti-fungal agent can comprise Abafungin, Albaconazole, Amorolfin, Amphotericin B, Anidulafungin, Bifonazole, Butenafine, Butoconazole, Candicidin, Caspofungin, Ciclopirox, Clotrimazole, Econazole, Fenticonazole, Filipin, Fluconazole, Flucytosine, Griseofulvin, Haloprogin, Hamycin, Isavuconazole, Isoconazole, Itraconazole, Ketoconazole, Micafungin, Miconazole, Naftifine, Natamycin, Nystatin, Omoconazole, Oxiconazole, Polygodial, Posaconazole, Ravuconazole, Rimocidin, Sertaconazole, Sulconazole, Terbinafine, Terconazole, Tioconazole, Tolnaftate, Undecylenic Acid, Voriconazole, or pharmaceutically acceptable salts thereof, or a combination thereof. In an aspect, an anti-fungal agent can be an azole. Azoles include, but are not limited to, the following: clotrimazole, econazole, fluconazole, itraconazole, ketoconazole, miconazole, oxiconazole, sulconazole, and voriconazole. As used herein, the recitation of an anti-fungal agent inherently encompasses the pharmaceutically acceptable salts thereof.

f. Anti-Viral Agents

Anti-viral agents are known to the art. As used herein, for example, an anti-viral can comprise Abacavir, Acyclovir (Aciclovir), Adefovir, Amantadine, Ampligen, Amprenavir (Agenerase), Umifenovir (Arbidol), Atazanavir, Atripla, Baloxavir marboxil (Xofluza), Biktarvy, Boceprevir, Bulevirtide, Cidofovir, Cobicistat (Tybost), Combivir, Daclatasvir (Daklinza), Darunavir, Delavirdine, Descovy, Didanosine, Docosanol, Dolutegravir, Doravirine (Pifeltro), Edoxudine, Efavirenz, Elvitegravir, Emtricitabine, Enfuvirtide, Entecavir, Etravirine (Intelence), Famciclovir, Fomivirsen, Fosamprenavir, Foscarnet, Ganciclovir (Cytovene), Ibacitabine, Ibalizumab (Trogarzo), Idoxuridine, Imiquimod, Imunovir, Indinavir, Lamivudine, Letermovir (Prevymis), Lopinavir, Loviride, Maraviroc, Methisazone, Moroxydine, Nelfinavir, Nevirapine, Nexavir (formerly Kutapressin), Nitazoxanide, Norvir, Oseltamivir (Tamiflu), Penciclovir, Peramivir, Penciclovir, Peramivir (Rapivab), Pleconaril, Podophyllotoxin, Raltegravir, Remdesivir, Ribavirin, Rilpivirine (Edurant), Rilpivirine, Rimantadine, Ritonavir, Saquinavir, Simeprevir (Olysio), Sofosbuvir, Stavudine, Taribavirin (Viramidine), Telaprevir, Telbivudine (Tyzeka), Tenofovir alafenamide, Tenofovir disoproxil, Tenofovir, Tipranavir, Trifluridine, Trizivir, Tromantadine, Truvada, Umifenovirk, Valaciclovir, Valganciclovir (Valtrex), Vicriviroc, Vidarabine, Zalcitabine, Zanamivir (Relenza), Zidovudine, and combinations thereof. As used herein, the recitation of any anti-viral agent inherently encompasses the pharmaceutically acceptable salts thereof.

g. Corticosteroids

Corticosteroids are well-known in the art. Corticosteroids mimic the effects of hormones that the body produces naturally in your adrenal glands. Corticosteroids can suppress inflammation and can reduce the signs and symptoms of inflammatory conditions (e.g., arthritis and asthma). Corticosteroids can also suppress the immune system. Corticosteroids can act on a number of different cells (e.g., mast cells, neutrophils, macrophages and lymphocytes) and a number of different mediators (e.g., histamine, leukotriene, and cytokine subtypes).

Steroids include, but are not limited to, the following: triamcinolone and its derivatives (e.g., diacetate, hexacetonide, and acetonide), betamethasone and its derivatives (e.g., dipropionate, benzoate, sodium phosphate, acetate, and valerate), dexamethasone and its derivatives (e.g., dipropionate and valerate), flunisolide, prednisone and its derivatives (e.g., acetate), prednisolone and its derivatives (e.g., acetate, sodium phosphate, and tebutate), methylprednisolone and its derivatives (e.g., acetate and sodium succinate), fluocinolone and its derivatives (e.g., acetonide), diflorasone and its derivatives (e.g., diacetate), halcinonide, desoximetasone (desoxymethasone), diflucortolone and its derivatives (e.g., valerate), flucloronide (fluclorolone acetonide), fluocinonide, fluocortolone, fluprednidene and its derivatives (e.g., acetate), flurandrenolide (flurandrenolone), clobetasol and its derivatives (e.g., propionate), clobetasone and its derivatives (e.g., butyrate), alclometasone, flumethasone and its derivatives (e.g., pivalate), fluocortolone and its derivatives (e.g., hexanoate), amcinonide, beclometasone and its derivatives (e.g., dipropionate), fluticasone and its derivatives (e.g., propionate), difluprednate, prednicarbate, flurandrenolide, mometasone, and desonide. As used herein, the recitation of a corticosteroid inherently encompasses the pharmaceutically acceptable salts thereof.

h. Analgesics

The compositions of the present disclosure can also be used in combination therapies with opioids and other analgesics, including narcotic analgesics, Mu receptor agonists, Kappa receptor antagonists, non-narcotic (i.e., non-addictive) analgesics, monoamine uptake inhibitors, adenosine regulating agents, cannabinoid derivatives, Substance P antagonists, neurokinin-1 receptor antagonists, calcitonin gene-related peptide (CGRP) and its receptor inhibitors, sodium channel blockers (e.g., local anesthetics, such as lidocaine, bupivacaine), and non-steroid anti-inflammatory drugs (NSAIDs) among others. Preferred combination therapies comprise a composition useful in methods described herein with one or more compounds selected from aceclofenac, acemetacin, α-acetamidocaproic acid, acetaminophen, acetaminosalol, acetanilide, acetylsalicylic acid (aspirin), S-adenosylmethionine, alclofenac, alfentanil, allylprodine. alminoprofen, aloxiprin, alphaprodine, aluminum bis (acetylsalicylate), amfenac, aminochlorthenoxazin, 3-amino-4-hydroxybutyric acid, 2-atnino-4-picoline, aminopropylon, aminopyrine, amixetrine, ammonium salicylate, ampiroxicam, amtolmetin guacil, anileridine, antipyrine, antipyrine salicylate, antrafenine, apazone, bendazac, benorylate, benoxaprofen, benzpiperylon, benzydamine, benzylmorphine, bermoprofen, bezitramide, a-bisabolol, bromfenac, p-bromoacetanilide, 5-bromosalicylic acid acetate, bromosaligenin, bucetin, bucloxic acid, bucolome, bufexamac, bumadizon, buprenorphine, butacetin, butibufen, butophanol, calcium acetylsalicylate, carbamazepine, carbiphene, carprofen, carsalam, chlorobutanol, chlorthenoxazin, choline salicylate, cinchophen, cinmetacin, ciramadol, clidanac, clometacin, clonitazene, clonixin, clopirac, clove, codeine, codeine methyl bromide, codeine phosphate, codeine sulfate, cropropamide, crotethamide, desomorphine, dexoxadrol, dextromoramide, dezocine, diampromide, diclofenac sodium, difenamizole, difenpiramide, diflunisal, dihydrocodeine, dihydrocodeinone enol acetate, dihydromorphine, dihydroxyalutninum acetylsalicylate, dimenoxadol, dimepheptanol, dimethylthiambutene, dioxaphetyl butyrate, dipipanone, diprocetyl, dipyrone, ditazol, droxicam, emorfazone, enfenamic acid, epirizole, eptazocine, etersalate, ethenzamide, ethoheptazine, ethoxazene, ethylmethylthiambutene, ethylmorphine, etodolac, etofenamate, etonitazene, eugenol, felbinac, fenbufen, fenclozic acid, fendosal, fenoprofen, fentanyl, fentiazac, fepradinol, feprazone, floctafenine, flufenamic acid, flunoxaprofen, fluoresone, flupirtine, fluproquazone, flurbiprofen, fosfosal, gentisic acid, glafenine, glucametacin, glycol salicylate, guaiazulene, hydrocodone, hydromorphone, hydroxypethidine, ibufenac, ibuprofen, ibuproxam, imidazole salicylate, indomethacin, indoprofen, isofezolac, isoladol, isomethadone, isonixin, isoxepac, isoxicam, ketobemidone, ketoprofen, ketorolac, p-lactophenetide, lefetamine, levorphanol, lofentanil, lonazolac, lomoxicam, loxoprofen, lysine acetylsalicylate, magnesium acetylsalicylate, meclofenamic acid, mefenamic acid, meperidine, meptazinol, mesalamine, metazocine, methadone hydrochloride, methotrimeprazine, metiazinic acid, metofoline, metopon, mofebutazone, mofezolac, morazone, morphine, morphine hydrochloride, morphine sulfate, morpholine salicylate, myrophine, nabumetone, nalbuphine, 1-naphthyl salicylate, naproxen, narceine, nefopam, nicomorphine, nifenazone, niflumic acid, nimesulide, 5′-nitro-2′-propoxyacetanilide, norlevorphanol, normethadone, normorphine, norpipanone, olsalazine, opium, oxaceprol, oxametacine, oxaprozin, oxycodone, oxymorphone, oxyphenbutazone, papaveretum, paranyline, parsalmide, pentazocine, perisoxal, phenacetin, phenadoxone, phenazocine, phenazopyridine hydrochloride, phenocoll, phenoperidine, phenopyrazone, phenyl acetylsalicylate, phenylbutazone, phenyl salicylate, phenyramidol, piketoprofen, piminodine, pipebuzone, piperylone, piprofen, pirazolac, piritramide, piroxicam, pranoprofen, proglumetacin, proheptazine, promedol, propacetamol, propiram, propoxyphene, propyphenazone, proquazone, protizinic acid, ramifenazone, remifentanil, rimazolium metilsulfate, salacetamide, salicin, salicylamide, salicylamide o-acetic acid, salicylsulfuric acid, salsalte, salverine, simetride, sodium salicylate, sufentanil, sulfasalazine, sulindac, superoxide dismutase, suprofen, suxibuzone, talniflumate, tenidap, tenoxicam, terofenamate, tetrandrine, thiazolinobutazone, tiaprofenic acid, tiaramide, tilidine, tinoridine, tolfenamic acid, tolmetin, tramadol, tropesin, viminol, xenbucin, ximoprofen, zaltoprofen and zomepirac. Analgesics are well known in the art. See, for example, The Merck Index, 12th Edition (1996), Therapeutic Category and Biological Activity Index, and the lists provided under “Analgesic”, “Anti-inflammatory” and “Antipyretic”. As used herein, the recitation of an analgesic inherently encompasses the pharmaceutically acceptable salts thereof.

i. Immunostimulants

The term “immunostimulant” is used herein to describe a substance which evokes, increases, and/or prolongs an immune response to an antigen. Immunomodulatory agents modulate the immune system, and, as used herein, immunostimulants are also referred to as immunomodulatory agents, where it is understood that the desired modulation is to stimulate the immune system. There are two main categories of immunostimulants, specific and non-specific. Specific immunostimulants provide antigenic specificity in immune response, such as vaccines or any antigen, and non-specific immunostimulants act irrespective of antigenic specificity to augment immune response of other antigen or stimulate components of the immune system without antigenic specificity, such as adjuvants and non-specific immunostimulators. Immunostimulants can include, but are not limited to, levamisole, thalidomide, erythemanodosum leprosum, BCG, cytokines such as interleukins or interferons, including recombinant cytokines and interleukin 2 (aldeslukin), 3D-MPL, QS21, CpG ODN 7909, miltefosine, anti-PD-1 or PD-LI targeting drugs, and acid (DCA, a macrophage stimulator), imiquimod and resiquimod (which activate immune cells through the toll-like receptor 7), chlorooxygen compounds such as tetrachlorodecaoxide (TCDO), agonistic CD40 antibodies, soluble CD40L, 4-1BB: 4-1BBL agonists, OX40 agonists, TLR agonists, moieties that deplete regulatory T cells, arabinitol-ceramide, glycerol-ceramide, 6-deoxy and 6-sulfono-myo-insitolceramide, iNKT agonists, and TLR agonists. As used herein, the recitation of an immunostimulant inherently encompasses the pharmaceutically acceptable salts thereof.

j. Immune-Based Product

As used herein, immune-based products include, but are not limited to, toll-like receptors modulators such as tlr1, tlr2, tlr3, tlr4, tlr5, tlr6, tlr7, tlr8, tlr9, tlr10, tlr11, tlr12, and tlr13:programmed cell death protein 1 (Pd-1) modulators; programmed death-ligand 1 (Pd-L1) modulators; IL-15 agonists; DermaVir; interleukin-7; plaquenil (hydroxychloroquine); proleukin (aldesleukin, IL-2); interferon alfa; interferon alfa-2b; interferon alfa-n3; pegylated interferon alfa; interferon gamma; hydroxyurea; mycophenolate mofetil (MPA) and its ester derivative mycophenolate mofetil (MMF); ribavirin; rintatolimod, polymer polyethyleneimine (PEI); gepon; rintatolimod; IL-12; WF-10; VGV-1; MOR-22; BMS-936559; CYT-107, interleukin-15/Fc fusion protein, normferon, peginterferon alfa-2a, peginterferon alfa-2b, recombinant interleukin-15, RPI-MN, GS-9620, and IR-103. As used herein, the recitation of an immune-based product inherently encompasses the pharmaceutically acceptable salts thereof.

### D. Compositions for Use in the Disclosed Methods

### 1. Compositions Having a NTSR1 Ligand

Disclosed herein is a composition comprising a therapeutically effective amount of a neurotensin receptor 1 (NTSR1) ligand or pharmaceutically acceptable salt, solvate, prodrug, or N-oxide thereof.

In an aspect, a disclosed NTSR1 ligand can comprise a functionally selective β-arrestin biased ligand.

In an aspect, a disclosed functionally selective β-arrestin biased ligand can comprise the general formula (I):

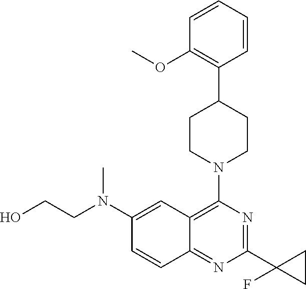

or a pharmaceutically acceptable salt, solvate, hydrate, prodrug, ester, or derivative thereof. In an aspect, a disclosed functionally selective β-arrestin biased ligand can comprise SBI-553.

In an aspect, a disclosed functionally selective β-arrestin biased ligand can comprise the general formula (II):

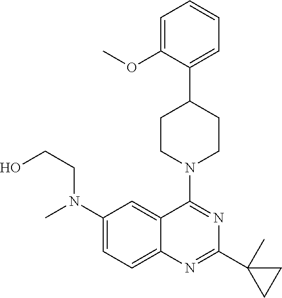

or a pharmaceutically acceptable salt, solvate, hydrate, prodrug, ester, or derivative thereof. In an aspect, a disclosed functionally selective β-arrestin biased ligand can comprise SBI-810.

In an aspect, a disclosed therapeutically effective amount of a NTSR1 ligand can comprise a dose of about 1 mg/kg/day to about 100 mg/kg/day. In an aspect, a therapeutically effective amount of a disclosed NTSR1 ligand can comprise a dose of about 1 mg/kg/day to about 20 mg/kg/day, about 20 mg/kg/day to about 40 mg/kg/day, about 40 mg/kg/day to about 60 mg/kg/day, about 60 mg/kg/day to about 80 mg/kg/day, or about 80 mg/kg/day to about 100 mg/kg/day. In an aspect, a therapeutically effective amount of a disclosed NTSR1 ligand can comprise a dose of about 1 mg/kg/day, about 5 mg/kg/day, about 10 mg/kg/day, about 15 mg/kg/day, about 20 mg/kg/day, about 25 mg/kg/day, about 30 mg/kg/day, about 35 mg/kg/day, about 40 mg/kg/day, about 45 mg/kg/day, about 50 mg/kg/day, about 55 mg/kg/day, about 60 mg/kg/day, about 65 mg/kg/day, about 70 mg/kg/day, about 75 mg/kg/day, about 80 mg/kg/day, about 85 mg/kg/day, about 90 mg/kg/day, about 95 mg/kg/day, or about 100 mg/kg/day. In an aspect, a therapeutically effective amount of a disclosed NTSR1 ligand can comprise a dose of more than 100 mg/kg/day.

In an aspect, a disclosed composition can further comprise a therapeutically effective amount of one or more therapeutic agents. A disclosed therapeutic agent can be any disclosed agent that effects a desired clinical outcome (e.g., biologically active agents, pharmaceutically active agents, chemotherapeutic agents, anti-bacterial agents, anti-fungal agents, anti-viral agents, corticosteroids, analgesics, immunostimulants, immune-based products, or any combination thereof) and are disclosed supra.

In an aspect, one or more therapeutic agents can comprise a STING agonist, a steroid, an opioid analgesic, a local anesthetic, an NSAID, a programmed cell death 1 ligand, an anti-cancer agent, and any combination thereof. In an aspect, a disclosed opioid analgesic can comprise codeine, dextropropyoxyphene, diamorphine, dihydrocodeine, meptazinol, methadone, morphine, nalbuphine, pentazocine, oxycodone, and any combination thereof.

In an aspect, a disclosed composition comprising a therapeutically effective amount of a NTSR1 ligand can treat and/or prevent pain in a subject. In an aspect, a disclosed composition comprising a therapeutically effective amount of a NTSR1 ligand can cause a subject to experience less pain, less intense pain, a shorter duration of pain, or a combination thereof.

In an aspect, a disclosed composition comprising a therapeutically effective amount of a NTSR1 ligand can improve a subject's ability to move and/or level of mobility. In an aspect, a disclosed composition comprising a therapeutically effective amount of a NTSR1 ligand can improve a subject's ability to move and/or level of mobility by about 10%, about 20%, about 30%, about 40%, about 50%, about 60%, about 70%, about 80%, about 90%, or about 100% as compared to native or control levels or to the subject's level of mobility prior to the administration of the disclosed composition. In an aspect, a disclosed composition can improve a subject's ability to move and/or level of mobility by about 10-20%, about 20-30%, about 30-40%, about 40-50%, about 50-60%, about 60-70%, about 70-80%, about 80-90%, or about 90-100% as compared to native or control levels or to the subject's ability to move and/or level of mobility prior to the administration of the disclosed composition. In an aspect, a disclosed composition can improve a subject's ability to move and/or level of mobility by about 0-25%, about 25-50%, about 50-75%, or about 75-100 as compared to native or control levels or to a subject's ability to move and/or level of mobility prior to the administration of the disclosed composition. In an aspect, a subject's ability to move and/or level of mobility prior to the administration of the disclosed composition can be used as a control.

In an aspect, a disclosed composition comprising a therapeutically effective amount of a NTSR1 ligand does not elicit hypothermia, hypotension, and/or motor impairment in a subject.

In an aspect, a disclosed composition or a disclosed pharmaceutical formulation comprising a therapeutically effective amount of a NTSR1 ligand can be formulated for oral administration, intravenous administration, intra-articular administration, epidural administration, intrathecal administration, intraperitoneal administration, or any combination thereof. In an aspect, a disclosed composition or a disclosed pharmaceutical formulation comprising a therapeutically effective amount of a NTSR1 ligand can be formulated for administration via topical administration (e.g., skin patch) or depot administration. In an aspect, a disclosed composition or a disclosed pharmaceutical formulation comprising a therapeutically effective amount of a NTSR1 ligand can be formulated for any route of administration disclosed herein.

In an aspect, a disclosed composition or a disclosed pharmaceutical formulation comprising a therapeutically effective amount of a NTSR1 ligand can be formulated for epidural administration (for example, by epidural injection) or for intra-articular (for example, by joint or vertebrate injection).

In an aspect, a disclosed composition comprising a therapeutically effective amount of a NTSR1 ligand can restore one or more aspects of cellular homeostasis and/or cellular functionality and/or metabolic dysregulation in a subject, such as, for example, a subject having acute and/or chronic pain. In an aspect, restoring one or more aspects of cellular homeostasis and/or cellular functionality can comprise reducing the subject's acute and/or chronic pain.

In an aspect, a disclosed composition comprising a therapeutically effective amount of a NTSR1 ligand can reduce a subject's pain by about 10%, about 20%, about 30%, about 40%, about 50%, about 60%, about 70%, about 80%, about 90%, or about 100% as compared to native or control levels or to the subject's level prior to the onset of pain (e.g., acute and/or chronic pain due to trauma, injury, cancer, disease and/or disorder, neuropathic pain, etc.). In an aspect, a disclosed composition comprising a therapeutically effective amount of a NTSR1 ligand can reduce a subject's pain by about 10-20%, about 20-30%, about 30-40%, about 40-50%, about 50-60%, about 60-70%, about 70-80%, about 80-90%, or about 90-100% as compared to native or control levels or to a subject's level prior to the onset of pain (e.g., acute and/or chronic pain due to trauma, injury, cancer, disease and/or disorder, neuropathic pain, etc.). In an aspect, a disclosed composition comprising a therapeutically effective amount of a NTSR1 ligand can reduce a subject's pain by about 0-25%, about 25-50%, about 50-75%, or about 75-100% as compared to native or control levels or to a subject's pain level prior to the onset of pain (e.g., acute and/or chronic pain due to trauma, injury, cancer, disease and/or disorder, neuropathic pain, etc.). In an aspect, a subject's pain level prior to the onset of pain can be used as a control.

In an aspect, a subject's acute and/or chronic pain can comprise back pain (including upper back pain and/or lower back pain), cancer pain, arthritis pain, headaches (including but not limited to migraines, cluster headaches, and tension headaches), neuralgias, neuropathies, pain due to a CNS injury, pain due to a PNS injury, pain with no known etiological cause, or any combination thereof. In an aspect, a subject's pain can be cancer pain. In an aspect, a subject's pain can be arthritis pain. In an aspect, a subject's pain can be headache pain. In an aspect, a subject's pain can be neuralgia pain. In an aspect, a subject's pain can be neuropathy pain. In an aspect, a subject's pain can be due to an injured sustained while serving in the armed forces or military.

Disclosed herein is a composition comprising a therapeutically effective amount of a neurotensin receptor 1 (NTSR1) ligand or pharmaceutically acceptable salt, solvate, prodrug, or N-oxide thereof, wherein the composition can treat and/or prevent pain in a subject.

Disclosed herein is a composition comprising a therapeutically effective amount of a neurotensin receptor 1 (NTSR1) ligand or pharmaceutically acceptable salt, solvate, prodrug, or N-oxide thereof, wherein the composition can cause the subject to experience less pain, less intense pain, a shorter duration of pain, or a combination thereof. Disclosed herein is a composition comprising a therapeutically effective amount of a neurotensin receptor 1 (NTSR1) ligand or pharmaceutically acceptable salt, solvate, prodrug, or N-oxide thereof, wherein the composition can improve the subject's ability to move and/or level of mobility. Disclosed herein is a composition comprising a therapeutically effective amount of a neurotensin receptor 1 (NTSR1) ligand or pharmaceutically acceptable salt, solvate, prodrug, or N-oxide thereof, wherein the composition does not elicit hypothermia, hypotension, and/or motor impairment in a subject. Disclosed herein is a composition comprising a therapeutically effective amount of a neurotensin receptor 1 (NTSR1) ligand or pharmaceutically acceptable salt, solvate, prodrug, or N-oxide thereof, wherein the composition does not elicit hypothermia, hypotension, and/or motor impairment in a subject, wherein the subject is in the military or is veteran.

Disclosed herein is a method of making a disclosed neurotensin receptor 1 (NTSR1) ligand or pharmaceutically acceptable salt, solvate, prodrug, or N-oxide thereof. In an aspect, methods of making and identifying a disclosed NTSR1 ligand or pharmaceutically acceptable salt, solvate, prodrug, or N-oxide thereof are provided by WO 2015/200534, which is incorporated by reference in its entirety for teaching the manufacturing of NTSR1 ligands including but not limited SBI-553 and SBI-810.

### 2. Compositions Having SBI-553

Disclosed herein is a composition comprising a therapeutically effective amount of SBI-553. Disclosed herein is a composition comprising a therapeutically effective amount of SBI-553, wherein the composition can treat and/or prevent pain in a subject. Disclosed herein is a composition comprising a therapeutically effective amount of SBI-553, wherein the composition does not elicit hypothermia, hypotension, and/or motor impairment in a subject.

In an aspect, a disclosed composition comprising a therapeutically effective amount of SBI-553 can further comprise a therapeutically effective amount of one or more therapeutic agents. A disclosed therapeutic agent can be any disclosed agent that effects a desired clinical outcome (e.g., biologically active agents, pharmaceutically active agents, chemotherapeutic agents, anti-bacterial agents, anti-fungal agents, anti-viral agents, corticosteroids, analgesics, immunostimulants, immune-based products, or any combination thereof) and are disclosed supra.

In an aspect, a disclosed composition comprising a therapeutically effective amount of SBI-553 can further comprise a STING agonist, a steroid, an opioid analgesic, a local anesthetic, an NSAID, a programmed cell death 1 ligand, an anti-cancer agent, and any combination thereof. In an aspect, a disclosed opioid analgesic can comprise codeine, dextropropyoxyphene. diamorphine, dihydrocodeine, meptazinol, methadone, morphine, nalbuphine, pentazocine, oxycodone, and any combination thereof.

In an aspect, a therapeutically effective amount of SBI-553 can comprise a dose of about 1 mg/kg/day to about 100 mg/kg/day. In an aspect, a therapeutically effective amount of SBI-553 can comprise a dose of about 1 mg/kg/day to about 20 mg/kg/day, about 20 mg/kg/day to about 40 mg/kg/day, about 40 mg/kg/day to about 60) mg/kg/day, about 60 mg/kg/day to about 80 mg/kg/day, or about 80 mg/kg/day to about 100 mg/kg/day. In an aspect, a therapeutically effective amount of SBI-553 can comprise a dose of about 1 mg/kg/day, about 5 mg/kg/day, about 10 mg/kg/day, about 15 mg/kg/day, about 20 mg/kg/day, about 25 mg/kg/day, about 30 mg/kg/day, about 35 mg/kg/day, about 40 mg/kg/day, about 45 mg/kg/day, about 50 mg/kg/day. about 55 mg/kg/day, about 60 mg/kg/day, about 65 mg/kg/day, about 70) mg/kg/day, about 75 mg/kg/day, about 80 mg/kg/day, about 85 mg/kg/day, about 90 mg/kg/day, about 95 mg/kg/day, or about 100 mg/kg/day. In an aspect, a therapeutically effective amount of SBI-553 can comprise a dose of more than 100 mg/kg/day.

In an aspect, a disclosed composition comprising a therapeutically effective amount of SBI-553 can treat and/or prevent pain in a subject. In an aspect, a disclosed composition comprising a therapeutically effective amount of SBI-553 can cause a subject to experience less pain, less intense pain, a shorter duration of pain, or a combination thereof.

In an aspect, a disclosed composition comprising a therapeutically effective amount of SBI-553 can improve a subject's ability to move and/or level of mobility. In an aspect, a disclosed composition comprising a therapeutically effective amount of SBI-553 can improve a subject's ability to move and/or level of mobility by about 10%, about 20%, about 30%, about 40%, about 50%, about 60%, about 70%, about 80%, about 90%, or about 100% as compared to native or control levels or to a subject's level of mobility prior to the administration of the disclosed composition comprising a therapeutically effective amount of SBI-553. In an aspect, a disclosed composition comprising a therapeutically effective amount of SBI-553 can improve a subject's ability to move and/or level of mobility by about 10-20%, about 20-30%, about 30-40%, about 40-50%, about 50-60%, about 60-70%, about 70-80%, about 80-90%, or about 90-100% as compared to native or control levels or to a subject's ability to move and/or level of mobility prior to the administration of the disclosed composition comprising a therapeutically effective amount of SBI-553. In an aspect, a disclosed composition comprising a therapeutically effective amount of SBI-553 can improve a subject's ability to move and/or level of mobility by about 0-25%, about 25-50%, about 50-75%, or about 75-100 as compared to native or control levels or to a subject's ability to move and/or level of mobility prior to the administration of the disclosed composition comprising a therapeutically effective amount of SBI-553. In an aspect, a subject's ability to move and/or level of mobility prior to the administration of a disclosed composition comprising a therapeutically effective amount of SBI-553 can be used as a control.

In an aspect, a disclosed composition comprising a therapeutically effective amount of SBI-553 does not elicit hypothermia, hypotension, and/or motor impairment in a subject.

In an aspect, a disclosed composition comprising a therapeutically effective amount of SBI-553 can be formulated for oral administration, intravenous administration, intra-articular administration, epidural administration, intrathecal administration, intraperitoneal administration, or any combination thereof. In an aspect, a disclosed composition comprising a therapeutically effective amount of SBI-553 can be formulated for administration via topical administration (e.g., skin patch) or depot administration. In an aspect, a disclosed composition comprising a therapeutically effective amount of SBI-553 can be formulated for any route of administration disclosed herein.

In an aspect, a disclosed composition comprising a therapeutically effective amount of SBI-553 can restore one or more aspects of cellular homeostasis and/or cellular functionality and/or metabolic dysregulation in a subject, such as, for example, a subject having acute and/or chronic pain. In an aspect, restoring one or more aspects of cellular homeostasis and/or cellular functionality can comprise reducing the subject's acute and/or chronic pain.

In an aspect, a disclosed composition comprising a therapeutically effective amount of SBI-553 can reduce a subject's pain by about 10%, about 20%, about 30%, about 40%, about 50%, about 60%, about 70%, about 80%, about 90%, or about 100% as compared to native or control levels or to the subject's level prior to the onset of pain (e.g., acute and/or chronic pain due to trauma, injury, cancer, disease and/or disorder, neuropathic pain, etc.). In an aspect, a disclosed composition comprising a therapeutically effective amount of SBI-553 can reduce a subject's pain by about 10-20%, about 20-30%, about 30-40%, about 40-50%, about 50-60%, about 60-70%, about 70-80%, about 80-90%, or about 90-100% as compared to native or control levels or to a subject's level prior to the onset of pain (e.g., acute and/or chronic pain due to trauma, injury, cancer, disease and/or disorder, neuropathic pain, etc.). In an aspect, a disclosed composition comprising a therapeutically effective amount of SBI-553 can reduce a subject's pain by about 0-25%, about 25-50%, about 50-75%, or about 75-100% as compared to native or control levels or to a subject's pain level prior to the onset of pain (e.g., acute and/or chronic pain due to trauma, injury, cancer, disease and/or disorder, neuropathic pain, etc.). In an aspect, a subject's pain level prior to the onset of pain can be used as a control.

In an aspect, a subject's acute and/or chronic pain can comprise back pain (including upper back pain and/or lower back pain), cancer pain, arthritis pain, headaches (including but not limited to migraines, cluster headaches, and tension headaches), neuralgias, neuropathies, pain due to a CNS injury, pain due to a PNS injury, pain with no known etiological cause, or any combination thereof. In an aspect, a subject's pain can be cancer pain. In an aspect, a subject's pain can be arthritis pain. In an aspect, a subject's pain can be headache pain. In an aspect, a subject's pain can be neuralgia pain. In an aspect, a subject's pain can be neuropathy pain. In an aspect, a subject's pain can be due to an injured sustained while serving in the armed forces or military.

Disclosed herein is a composition comprising a therapeutically effective amount of SBI-553, wherein the composition can cause the subject to experience less pain, less intense pain, a shorter duration of pain, or a combination thereof. Disclosed herein is a composition comprising a therapeutically effective amount of SBI-553, wherein the composition can improve the subject's ability to move and/or level of mobility. Disclosed herein is a composition comprising a therapeutically effective amount of SBI-553, wherein the composition does not elicit hypothermia, hypotension, and/or motor impairment in a subject. Disclosed herein is a composition comprising a therapeutically effective amount of SBI-553, wherein the composition does not elicit hypothermia, hypotension, and/or motor impairment in a subject, wherein the subject is in the military or is veteran.

Disclosed herein is a method of making SBI-553. In an aspect, methods of making and identifying SBI-553 are provided by WO 2015/200534, which is incorporated by reference in its entirety for teaching the manufacturing of SBI-553.

### 3. Compositions Having SBI-810

Disclosed herein is a composition comprising a therapeutically effective amount of SBI-810. Disclosed herein is a composition comprising a therapeutically effective amount of SBI-810, wherein the composition can treat and/or prevent pain in a subject. Disclosed herein is a composition comprising a therapeutically effective amount of SBI-810, wherein the composition does not elicit hypothermia, hypotension, and/or motor impairment in a subject.

In an aspect, a disclosed composition comprising a therapeutically effective amount of SBI-810 can further comprise a therapeutically effective amount of one or more therapeutic agents. A disclosed therapeutic agent can be any disclosed agent that effects a desired clinical outcome (e.g., biologically active agents, pharmaceutically active agents, chemotherapeutic agents, anti-bacterial agents, anti-fungal agents, anti-viral agents, corticosteroids, analgesics, immunostimulants, immune-based products, or any combination thereof) and are disclosed supra. In an aspect, a disclosed composition comprising a therapeutically effective amount of SBI-810 can further comprise a STING agonist, a steroid, an opioid analgesic, a local anesthetic, an NSAID, a programmed cell death I ligand, an anti-cancer agent, and any combination thereof. In an aspect, a disclosed opioid analgesic can comprise codeine, dextropropy oxyphene, diamorphine, dihydrocodeine, meptazinol, methadone, morphine, nalbuphine, pentazocine, oxycodone, and any combination thereof.

In an aspect, a therapeutically effective amount of SBI-810 can comprise a dose of about 1 mg/kg/day to about 100 mg/kg/day. In an aspect, a therapeutically effective amount of SBI-810 can comprise a dose of about 1 mg/kg/day to about 20 mg/kg/day, about 20 mg/kg/day to about 40 mg/kg/day, about 40 mg/kg/day to about 60 mg/kg/day, about 60 mg/kg/day to about 80 mg/kg/day, or about 80 mg/kg/day to about 100 mg/kg/day. In an aspect, a therapeutically effective amount of SBI-810 can comprise a dose of about 1 mg/kg/day, about 5 mg/kg/day, about 10 mg/kg/day, about 15 mg/kg/day, about 20 mg/kg/day, about 25 mg/kg/day, about 30 mg/kg/day, about 35 mg/kg/day, about 40 mg/kg/day, about 45 mg/kg/day, about 50 mg/kg/day, about 55 mg/kg/day, about 60 mg/kg/day, about 65 mg/kg/day, about 70 mg/kg/day, about 75 mg/kg/day, about 80 mg/kg/day, about 85 mg/kg/day, about 90 mg/kg/day, about 95 mg/kg/day, or about 100 mg/kg/day. In an aspect, a therapeutically effective amount of SBI-810 can comprise a dose of more than 100 mg/kg/day.

In an aspect, a disclosed composition comprising a therapeutically effective amount of SBI-810 can treat and/or prevent pain in a subject. In an aspect, a disclosed composition comprising a therapeutically effective amount of SBI-810 can cause a subject to experience less pain, less intense pain, a shorter duration of pain, or a combination thereof.

In an aspect, a disclosed composition comprising a therapeutically effective amount of SBI-810 can improve a subject's ability to move and/or level of mobility. In an aspect, a disclosed composition comprising a therapeutically effective amount of SBI-810 can improve a subject's ability to move and/or level of mobility by about 10%, about 20%, about 30%, about 40%, about 50%, about 60%, about 70%, about 80%, about 90%, or about 100% as compared to native or control levels or to a subject's level of mobility prior to the administration of the disclosed composition comprising a therapeutically effective amount of SBI-810. In an aspect, a disclosed composition comprising a therapeutically effective amount of SBI-810 can improve a subject's ability to move and/or level of mobility by about 10-20%, about 20-30%, about 30-40%, about 40-50%, about 50-60%, about 60-70%, about 70-80%, about 80-90%, or about 90-100% as compared to native or control levels or to a subject's ability to move and/or level of mobility prior to the administration of the disclosed composition comprising a therapeutically effective amount of SBI-810. In an aspect, a disclosed composition comprising a therapeutically effective amount of SBI-810 can improve a subject's ability to move and/or level of mobility by about 0-25%, about 25-50%, about 50-75%, or about 75-100 as compared to native or control levels or to a subject's ability to move and/or level of mobility prior to the administration of the disclosed composition comprising a therapeutically effective amount of SBI-810. In an aspect, a subject's ability to move and/or level of mobility prior to the administration of a disclosed composition comprising a therapeutically effective amount of SBI-810 can be used as a control.

In an aspect, a disclosed composition does not elicit hypothermia, hypotension, and/or motor impairment in a subject.

In an aspect, a disclosed composition comprising a therapeutically effective amount of SBI-810 can be prepared for systemic or direct administration. In an aspect, a disclosed composition comprising a therapeutically effective amount of SBI-810 can be prepared for any disclosed method of administration. In an aspect, a disclosed composition comprising a therapeutically effective amount of SBI-810 can be prepared for oral administration, intrathecal administration, intravenous administration, intra-articular administration, epidural administration, intraperitoneal administration, or any combination thereof. In an aspect, a disclosed composition comprising a therapeutically effective amount of SBI-810 can be formulated for administration via topical administration (e.g., skin patch) or depot administration. In an aspect, a disclosed composition comprising a therapeutically effective amount of SBI-810 can be prepared for any method of administration disclosed herein. In an aspect, a disclosed composition comprising a therapeutically effective amount of SBI-810 can be prepared for administration via multiple routes either concurrently or sequentially. For example, in an aspect, a disclosed composition comprising a therapeutically effective amount of SBI-810 can be first administered orally and then be administered intravenously. In an aspect, a disclosed composition comprising a therapeutically effective amount of SBI-810 can be first administered intravenously and then be administered orally. A skilled clinical can determine the best route of administration for a subject at a given time.

In an aspect, a disclosed composition comprising a therapeutically effective amount of SBI-810 can be formulated for oral administration, intravenous administration, intra-articular administration, epidural administration, intrathecal administration, intraperitoneal administration, or any combination thereof. In an aspect, a disclosed composition comprising a therapeutically effective amount of SBI-810 can be formulated for administration via topical administration (e.g., skin patch) or depot administration. In an aspect a disclosed composition comprising a therapeutically effective amount of SBI-810 can be formulated for any route of administration disclosed herein.

In an aspect, a disclosed composition comprising a therapeutically effective amount of SBI-810 can restore one or more aspects of cellular homeostasis and/or cellular functionality and/or metabolic dysregulation in a subject, such as, for example, a subject having acute and/or chronic pain. In an aspect, restoring one or more aspects of cellular homeostasis and/or cellular functionality can comprise reducing a subject's acute and/or chronic pain.

In an aspect, a disclosed composition comprising a therapeutically effective amount of SBI-810 can reduce a subject's pain by about 10%, about 20%, about 30%, about 40%, about 50%, about 60%, about 70%, about 80%, about 90%, or about 100% as compared to native or control levels or to the subject's level prior to the onset of pain (e.g., acute and/or chronic pain due to trauma, injury, cancer, disease and/or disorder, neuropathic pain, etc.). In an aspect, a disclosed composition comprising a therapeutically effective amount of SBI-810 can reduce a subject's pain by about 10-20%, about 20-30%, about 30-40%, about 40-50%, about 50-60%, about 60-70%, about 70-80%, about 80-90%, or about 90-100% as compared to native or control levels or to a subject's level prior to the onset of pain (e.g., acute and/or chronic pain due to trauma, injury, cancer, disease and/or disorder, neuropathic pain, etc.). In an aspect, a disclosed composition comprising a therapeutically effective amount of SBI-810 can reduce a subject's pain by about 0-25%, about 25-50%, about 50-75%, or about 75-100% as compared to native or control levels or to a subject's pain level prior to the onset of pain (e.g., acute and/or chronic pain due to trauma, injury, cancer, disease and/or disorder, neuropathic pain, etc.). In an aspect, a subject's pain level prior to the onset of pain can be used as a control.

In an aspect, a subject's acute and/or chronic pain can comprise back pain (including upper back pain and/or lower back pain), cancer pain, arthritis pain, headaches (including but not limited to migraines, cluster headaches, and tension headaches), neuralgias, neuropathies, pain due to a CNS injury, pain due to a PNS injury, pain with no known etiological cause, or any combination thereof. In an aspect, a subject's pain can be cancer pain. In an aspect, a subject's pain can be arthritis pain. In an aspect, a subject's pain can be headache pain. In an aspect, a subject's pain can be neuralgia pain. In an aspect, a subject's pain can be neuropathy pain. In an aspect, a subject's pain can be due to an injured sustained while serving in the armed forces or military.

Disclosed herein is a composition comprising a therapeutically effective amount of SBI-810, wherein the composition can cause the subject to experience less pain, less intense pain, a shorter duration of pain, or a combination thereof. Disclosed herein is a composition comprising a therapeutically effective amount of SBI-810, wherein the composition can improve the subject's ability to move and/or level of mobility. Disclosed herein is a composition comprising a therapeutically effective amount of SBI-810, wherein the composition does not elicit hypothermia, hypotension, and/or motor impairment in a subject. Disclosed herein is a composition comprising a therapeutically effective amount of SBI-810, wherein the composition does not elicit hypothermia, hypotension, and/or motor impairment in a subject, wherein the subject is in the military or is veteran.

Disclosed herein is a method of making SBI-810. In an aspect, methods of making and identifying SBI-810 are provided by WO 2015/200534, which is incorporated by reference in its entirety for teaching the manufacturing of SBI-810.

### 4. Formulations Having NTSR1 Ligands

Disclosed herein is a pharmaceutical formulation comprising a therapeutically effective amount of a neurotensin receptor 1 (NTSR1) ligand or pharmaceutically acceptable salt, solvate, prodrug, or N-oxide thereof, and a disclosed and a pharmaceutically acceptable carrier.

In an aspect of a disclosed pharmaceutical formulation, a disclosed NTSR1 ligand can comprise a functionally selective β-arrestin biased ligand.

In an aspect of a disclosed pharmaceutical formulation, a disclosed functionally selective β-arrestin biased ligand can comprise the general formula (I):

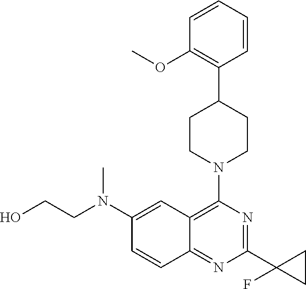

or a pharmaceutically acceptable salt, solvate, hydrate, prodrug, ester, or derivative thereof. In an aspect of a disclosed pharmaceutical formulation, a disclosed functionally selective β-arrestin biased ligand can comprise SBI-553.

In an aspect of a disclosed pharmaceutical formulation, a disclosed functionally selective β-arrestin biased ligand can comprise the general formula (II):

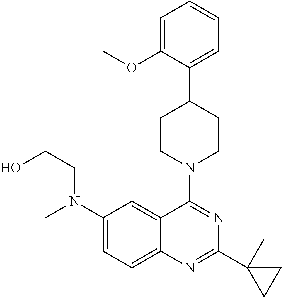

or a pharmaceutically acceptable salt, solvate, hydrate, prodrug, ester, or derivative thereof. In an aspect of a disclosed pharmaceutical formulation, a disclosed functionally selective β-arrestin biased ligand can comprise SBI-810.

In an aspect of a disclosed pharmaceutical formulation, a therapeutically effective amount of the NTSR1 ligand can comprise a dose of about 1 mg/kg/day to about 100 mg/kg/day. In an aspect of a disclosed pharmaceutical formulation, a therapeutically effective amount of the NTSR1 ligand can comprise a dose of about 1 mg/kg/day to about 20 mg/kg/day, about 20 mg/kg/day to about 40 mg/kg/day, about 40 mg/kg/day to about 60 mg/kg/day, about 60 mg/kg/day to about 80 mg/kg/day, or about 80 mg/kg/day to about 100 mg/kg/day. In an aspect of a disclosed pharmaceutical formulation, a therapeutically effective amount of the NTSR1 ligand can comprise a dose of about 1 mg/kg/day, about 5 mg/kg/day, about 10 mg/kg/day, about 15 mg/kg/day, about 20 mg/kg/day, about 25 mg/kg/day, about 30 mg/kg/day, about 35 mg/kg/day, about 40 mg/kg/day, about 45 mg/kg/day, about 50 mg/kg/day, about 55 mg/kg/day, about 60 mg/kg/day, about 65 mg/kg/day, about 70 mg/kg/day, about 75 mg/kg/day, about 80 mg/kg/day, about 85 mg/kg/day, about 90 mg/kg/day, about 95 mg/kg/day, or about 100 mg/kg/day. In an aspect of a disclosed pharmaceutical formulation, a therapeutically effective amount of the NTSR1 ligand can comprise a dose of more than 100 mg/kg/day.

In an aspect, a disclosed pharmaceutical formulation can further comprise a therapeutically effective amount of one or more therapeutic agents. A disclosed therapeutic agent can be any disclosed agent that effects a desired clinical outcome (e.g., biologically active agents, pharmaceutically active agents, chemotherapeutic agents, anti-bacterial agents, anti-fungal agents, anti-viral agents, corticosteroids, analgesics, immunostimulants, immune-based products, or any combination thereof) and are disclosed supra.

In an aspect of a disclosed pharmaceutical formulation, one or more therapeutic agents can comprise a STING agonist, a steroid, an opioid analgesic, a local anesthetic, an NSAID, a programmed cell death 1 ligand, an anti-cancer agent, and any combination thereof. In an aspect, a disclosed opioid analgesic can comprise codeine, dextropropyoxyphene, diamorphine, dihydrocodeine, meptazinol, methadone, morphine, nalbuphine, pentazocine, oxycodone, and any combination thereof.

In an aspect, a disclosed pharmaceutical formulation comprising a therapeutically effective amount of a NTSR1 ligand can treat and/or prevent pain in a subject. In an aspect, a disclosed pharmaceutical formulation can cause a subject to experience less pain, less intense pain, a shorter duration of pain, or a combination thereof.

In an aspect, a disclosed pharmaceutical formulation comprising a therapeutically effective amount of a NTSR1 ligand can improve a subject's ability to move and/or level of mobility. In an aspect, a disclosed pharmaceutical formulation comprising a therapeutically effective amount of a NTSR1 ligand can improve a subject's ability to move and/or level of mobility by about 10%, about 20%, about 30%, about 40%, about 50%, about 60%, about 70%, about 80%, about 90%, or about 100% as compared to native or control levels or to a subject's level of mobility prior to the administration of the disclosed pharmaceutical formulation. In an aspect, a disclosed pharmaceutical formulation can improve the subject's ability to move and/or level of mobility by about 10-20%, about 20-30%, about 30-40%, about 40-50%, about 50-60%, about 60-70%, about 70-80%, about 80-90%, or about 90-100% as compared to native or control levels or to a subject's ability to move and/or level of mobility prior to the administration of the disclosed pharmaceutical formulation. In an aspect, a disclosed pharmaceutical formulation can improve a subject's ability to move and/or level of mobility by about 0-25%, about 25-50%, about 50-75%, or about 75-100as compared to native or control levels or to a subject's ability to move and/or level of mobility prior to the administration of the disclosed pharmaceutical formulation. In an aspect, a subject's ability to move and/or level of mobility prior to the administration of a disclosed pharmaceutical formulation can be used as a control.

In an aspect, a disclosed pharmaceutical formulation comprising a therapeutically effective amount of a NTSR1 ligand does not elicit hypothermia, hypotension, and/or motor impairment in a subject.

In an aspect, a disclosed pharmaceutical formulation comprising a therapeutically effective amount of a NTSR1 ligand can be prepared for systemic or direct administration. In an aspect, a disclosed pharmaceutical formulation comprising a therapeutically effective amount of a NTSR1 ligand can be prepared for any disclosed method of administration. In an aspect, a disclosed pharmaceutical formulation comprising a therapeutically effective amount of a NTSR1 ligand can be prepared for oral administration, intrathecal administration, intravenous administration, intra-articular administration, epidural administration, intraperitoneal administration, or any combination thereof. In an aspect, a disclosed pharmaceutical formulation comprising a therapeutically effective amount of a NTSR1 ligand can be formulated for administration via topical administration (e.g., skin patch) or depot administration. In an aspect, a disclosed pharmaceutical formulation comprising a therapeutically effective amount of a NTSR1 ligand can be prepared for any method of administration disclosed herein. In an aspect, a disclosed pharmaceutical formulation comprising a therapeutically effective amount of a NTSR1 ligand can be prepared for administration via multiple routes either concurrently or sequentially. For example, in an aspect, a disclosed pharmaceutical formulation comprising a therapeutically effective amount of a NTSR1 ligand can be first administered orally and then be administered intravenously. In an aspect, a disclosed pharmaceutical formulation comprising a therapeutically effective amount of a NTSR1 ligand can be first administered intravenously and then be administered orally. A skilled clinical can determine the best route of administration for a subject at a given time.

In an aspect, a disclosed pharmaceutical formulation comprising a therapeutically effective amount of a NTSR1 ligand can be formulated for oral administration, intravenous administration, intra-articular administration, epidural administration, intrathecal administration, intraperitoneal administration, or any combination thereof. In an aspect, a disclosed pharmaceutical formulation comprising a therapeutically effective amount of a NTSR1 ligand can be formulated for administration via topical administration (e.g., skin patch) or depot administration. In an aspect a disclosed pharmaceutical formulation comprising a therapeutically effective amount of a NTSR1 ligand can be formulated for any route of administration disclosed herein.

In an aspect, a disclosed pharmaceutical formulation comprising a therapeutically effective amount of a NTSR1 ligand can restore one or more aspects of cellular homeostasis and/or cellular functionality and/or metabolic dysregulation in a subject, such as, for example, a subject having acute and/or chronic pain. In an aspect, restoring one or more aspects of cellular homeostasis and/or cellular functionality can comprise reducing a subject's acute and/or chronic pain.

In an aspect, a disclosed pharmaceutical formulation comprising a therapeutically effective amount of a NTSR1 ligand can reduce a subject's pain by about 10%, about 20%, about 30%. about 40%, about 50%, about 60%, about 70%, about 80%, about 90%, or about 100% as compared to native or control levels or to the subject's level prior to the onset of pain (e.g., acute and/or chronic pain due to trauma, injury, cancer, disease and/or disorder, neuropathic pain, etc.). In an aspect, a disclosed pharmaceutical formulation comprising a therapeutically effective amount of a NTSR1 ligand can reduce a subject's pain by about 10-20%, about 20-30%, about 30-40%, about 40-50%, about 50-60%, about 60-70%, about 70-80%, about 80-90%, or about 90-100% as compared to native or control levels or to a subject's level prior to the onset of pain (e.g., acute and/or chronic pain due to trauma, injury, cancer, disease and/or disorder, neuropathic pain. etc.). In an aspect, a disclosed pharmaceutical formulation comprising a therapeutically effective amount of a NTSR1 ligand can reduce a subject's pain by about 0-25%, about 25-50%, about 50-75%, or about 75-100% as compared to native or control levels or to a subject's pain level prior to the onset of pain (e.g., acute and/or chronic pain due to trauma, injury, cancer, disease and/or disorder, neuropathic pain, etc.). In an aspect, a subject's pain level prior to the onset of pain can be used as a control.

In an aspect, a subject's acute and/or chronic pain can comprise back pain (including upper back pain and/or lower back pain), cancer pain, arthritis pain, headaches (including but not limited to migraines, cluster headaches, and tension headaches), neuralgias, neuropathies, pain due to a CNS injury, pain due to a PNS injury, pain with no known etiological cause, or any combination thereof. In an aspect, a subject's pain can be cancer pain. In an aspect, a subject's pain can be arthritis pain. In an aspect, a subject's pain can be headache pain. In an aspect, a subject's pain can be neuralgia pain. In an aspect, a subject's pain can be neuropathy pain. In an aspect, a subject's pain can be due to an injured sustained while serving in the armed forces or military.

Disclosed herein is a pharmaceutical formulation comprising a therapeutically effective amount of a neurotensin receptor 1 (NTSR1) ligand or pharmaceutically acceptable salt, solvate, prodrug, or N-oxide thereof, wherein the composition can cause the subject to experience less pain, less intense pain, a shorter duration of pain, or a combination thereof. Disclosed herein is a pharmaceutical formulation comprising a therapeutically effective amount of a neurotensin receptor 1 (NTSR1) ligand or pharmaceutically acceptable salt, solvate, prodrug, or N-oxide thereof, wherein the composition can improve the subject's ability to move and/or level of mobility. Disclosed herein is a pharmaceutical formulation comprising a therapeutically effective amount of a neurotensin receptor 1 (NTSR1) ligand or pharmaceutically acceptable salt, solvate, prodrug, or N-oxide thereof, wherein the composition does not elicit hypothermia, hypotension, and/or motor impairment in a subject. Disclosed herein is a pharmaceutical formulation comprising a therapeutically effective amount of a neurotensin receptor 1 (NTSR1) ligand or pharmaceutically acceptable salt, solvate, prodrug, or N-oxide thereof, wherein the composition does not elicit hypothermia, hypotension, and/or motor impairment in a subject, wherein the subject is in the military or is veteran.

### 5. Formulations Having SBI-553

Disclosed herein is a pharmaceutical formulation comprising a therapeutically effective amount of SBI-553. Disclosed herein is a pharmaceutical formulation comprising a therapeutically effective amount of SBI-553, wherein the pharmaceutical formulation can treat and/or prevent pain in a subject. Disclosed herein is a pharmaceutical formulation comprising a therapeutically effective amount of SBI-553, wherein the pharmaceutical formulation does not elicit hypothermia, hypotension, and/or motor impairment in a subject.

In an aspect of a disclosed pharmaceutical formulation, a therapeutically effective amount of SBI-553 can comprise a dose of about 1 mg/kg/day to about 100 mg/kg/day. In an aspect of a disclosed pharmaceutical formulation, a therapeutically effective amount of SBI-553 can comprise a dose of about 1 mg/kg/day to about 20 mg/kg/day, about 20 mg/kg/day to about 40 mg/kg/day, about 40 mg/kg/day to about 60 mg/kg/day, about 60 mg/kg/day to about 80 mg/kg/day, or about 80 mg/kg/day to about 100 mg/kg/day. In an aspect of a disclosed pharmaceutical formulation, a therapeutically effective amount of SBI-553 can comprise a dose of about 1 mg/kg/day, about 5 mg/kg/day, about 10 mg/kg/day, about 15 mg/kg/day, about 20 mg/kg/day, about 25 mg/kg/day, about 30) mg/kg/day, about 35 mg/kg/day, about 40 mg/kg/day, about 45 mg/kg/day, about 50 mg/kg/day, about 55 mg/kg/day, about 60 mg/kg/day, about 65 mg/kg/day, about 70 mg/kg/day, about 75 mg/kg/day, about 80 mg/kg/day, about 85 mg/kg/day, about 90 mg/kg/day, about 95 mg/kg/day, or about 100 mg/kg/day. In an aspect of a disclosed pharmaceutical formulation, a therapeutically effective amount of SBI-553 can comprise a dose of more than 100 mg/kg/day.

In an aspect, a disclosed pharmaceutical formulation comprising a therapeutically effective amount of SBI-553 can further comprise a therapeutically effective amount of one or more therapeutic agents. A disclosed therapeutic agent can be any disclosed agent that effects a desired clinical outcome (e.g., biologically active agents, pharmaceutically active agents, chemotherapeutic agents, anti-bacterial agents, anti-fungal agents, anti-viral agents, corticosteroids, analgesics, immunostimulants, immune-based products, or any combination thereof) and are disclosed supra.

In an aspect, a disclosed pharmaceutical formulation comprising a therapeutically effective amount of SBI-553 can further comprise a STING agonist, a steroid, an opioid analgesic, a local anesthetic, an NSAID, a programmed cell death 1 ligand, an anti-cancer agent, and any combination thereof. In an aspect, a disclosed opioid analgesic can comprise codeine, dextropropyoxyphene, diamorphine, dihydrocodeine, meptazinol, methadone, morphine, nalbuphine, pentazocine, oxycodone, and any combination thereof.

In an aspect, a disclosed pharmaceutical formulation comprising a therapeutically effective amount of SBI-553 can treat and/or prevent pain in a subject. In an aspect, a disclosed pharmaceutical formulation comprising a therapeutically effective amount of SBI-553 can cause the subject to experience less pain, less intense pain, a shorter duration of pain, or a combination thereof.

In an aspect, a disclosed pharmaceutical formulation comprising a therapeutically effective amount of SBI-553 can improve the subject's ability to move and/or level of mobility. In an aspect, a disclosed pharmaceutical formulation comprising a therapeutically effective amount of SBI-553 can improve the subject's ability to move and/or level of mobility by about 10%, about 20%, about 30%, about 40%, about 50%, about 60%, about 70%, about 80%, about 90%, or about 100% as compared to native or control levels or to the subject's level of mobility prior to the administration of the disclosed pharmaceutical formulation comprising a therapeutically effective amount of SBI-553. In an aspect, a disclosed pharmaceutical formulation comprising a therapeutically effective amount of SBI-553 can improve the subject's ability to move and/or level of mobility by about 10-20%, about 20-30%, about 30-40%, about 40-50%, about 50-60%, about 60-70%, about 70-80%, about 80-90%, or about 90-100% as compared to native or control levels or to the subject's ability to move and/or level of mobility prior to the administration of the disclosed pharmaceutical formulation comprising a therapeutically effective amount of SBI-553. In an aspect, a disclosed pharmaceutical formulation comprising a therapeutically effective amount of SBI-553 can improve the subject's ability to move and/or level of mobility by about 0-25%, about 25-50%, about 50-75%, or about 75-100 as compared to native or control levels or to the subject's ability to move and/or level of mobility prior to the administration of the disclosed pharmaceutical formulation comprising a therapeutically effective amount of SBI-553. In an aspect, the subject's ability to move and/or level of mobility prior to the administration of a disclosed pharmaceutical formulation comprising a therapeutically effective amount of SBI-553 can be used as a control.

In an aspect, a disclosed pharmaceutical formulation comprising a therapeutically effective amount of SBI-553 does not elicit hypothermia, hypotension, and/or motor impairment in a subject.

In an aspect, a disclosed pharmaceutical formulation comprising a therapeutically effective amount of SBI-553 can be prepared for systemic or direct administration. In an aspect, a disclosed pharmaceutical formulation comprising a therapeutically effective amount of SBI-553 can be prepared for any disclosed method of administration. In an aspect, a disclosed pharmaceutical formulation comprising a therapeutically effective amount of SBI-553 can be prepared for oral administration, intrathecal administration, intravenous administration, intra-articular administration, epidural administration, intratumoral administration, intraperitoneal administration, or any combination thereof. In an aspect, a disclosed pharmaceutical formulation comprising a therapeutically effective amount of SBI-553 can be formulated for administration via topical administration (e.g., skin patch) or depot administration. In an aspect, a disclosed pharmaceutical formulation comprising a therapeutically effective amount of SBI-553 can be prepared for any method of administration disclosed herein. In an aspect, a disclosed pharmaceutical formulation comprising a therapeutically effective amount of SBI-553 can be prepared for administration via multiple routes either concurrently or sequentially. For example, in an aspect, a disclosed pharmaceutical formulation comprising a therapeutically effective amount of SBI-553 can be first administered orally and then be administered intravenously. In an aspect, a disclosed pharmaceutical formulation comprising a therapeutically effective amount of SBI-553 can be first administered intravenously and then be administered orally. A skilled clinical can determine the best route of administration for a subject at a given time.

In an aspect, a disclosed pharmaceutical formulation comprising a therapeutically effective amount of SBI-553 can be formulated for oral administration, intravenous administration, intra-articular administration, epidural administration, intrathecal administration, intraperitoneal administration, or any combination thereof.

In an aspect, a disclosed pharmaceutical formulation comprising a therapeutically effective amount of SBI-553 can restore one or more aspects of cellular homeostasis and/or cellular functionality and/or metabolic dysregulation in a subject, such as, for example, a subject having acute and/or chronic pain. In an aspect, restoring one or more aspects of cellular homeostasis and/or cellular functionality can comprise reducing the subject's acute and/or chronic pain.

In an aspect, a disclosed pharmaceutical formulation comprising a therapeutically effective amount of SBI-553 can reduce a subject's pain by about 10%, about 20%, about 30%, about 40%, about 50%, about 60%, about 70%, about 80%, about 90%, or about 100% as compared to native or control levels or to the subject's level prior to the onset of pain (e.g., acute and/or chronic pain due to trauma, injury, cancer, disease and/or disorder, neuropathic pain, etc.). In an aspect, a disclosed pharmaceutical formulation comprising a therapeutically effective amount of SBI-553can reduce a subject's pain by about 10-20%, about 20-30%, about 30-40%, about 40-50%, about 50-60%, about 60-70%, about 70-80%, about 80-90%, or about 90-100% as compared to native or control levels or to a subject's level prior to the onset of pain (e.g., acute and/or chronic pain due to trauma, injury, cancer, disease and/or disorder, neuropathic pain, etc.). In an aspect, a disclosed pharmaceutical formulation comprising a therapeutically effective amount of SBI-553 can reduce a subject's pain by about 0-25%, about 25-50%, about 50-75%, or about 75-100% as compared to native or control levels or to a subject's pain level prior to the onset of pain (e.g., acute and/or chronic pain due to trauma, injury, cancer, disease and/or disorder, neuropathic pain, etc.). In an aspect, a subject's pain level prior to the onset of pain can be used as a control.

In an aspect, a subject's acute and/or chronic pain can comprise back pain (including upper back pain and/or lower back pain), cancer pain, arthritis pain, headaches (including but not limited to migraines, cluster headaches, and tension headaches), neuralgias, neuropathies, pain due to a CNS injury, pain due to a PNS injury, pain with no known etiological cause, or any combination thereof. In an aspect, a subject's pain can be cancer pain. In an aspect, a subject's pain can be arthritis pain. In an aspect, a subject's pain can be headache pain. In an aspect, a subject's pain can be neuralgia pain. In an aspect, a subject's pain can be neuropathy pain. In an aspect, a subject's pain can be due to an injured sustained while serving in the armed forces or military.

Disclosed herein is a pharmaceutical formulation comprising a therapeutically effective amount of SBI-533, wherein the composition can cause the subject to experience less pain, less intense pain, a shorter duration of pain, or a combination thereof. Disclosed herein is a pharmaceutical formulation comprising a therapeutically effective amount of SBI-533, wherein the composition can improve the subject's ability to move and/or level of mobility. Disclosed herein is a pharmaceutical formulation comprising a therapeutically effective amount of SBI-533, wherein the composition does not elicit hypothermia, hypotension, and/or motor impairment in a subject. Disclosed herein is a pharmaceutical formulation comprising a therapeutically effective amount of SBI-533, wherein the composition does not elicit hypothermia, hypotension, and/or motor impairment in a subject, wherein the subject is in the military or is veteran.

### 6. Formulations Having SBI-810

Disclosed herein is a pharmaceutical formulation comprising a therapeutically effective amount of SBI-810. Disclosed herein is a pharmaceutical formulation comprising a therapeutically effective amount of SBI-810, wherein the pharmaceutical formulation can treat and/or prevent pain in a subject. Disclosed herein is a pharmaceutical formulation comprising a therapeutically effective amount of SBI-810, wherein the composition does not elicit hypothermia, hypotension, and/or motor impairment in a subject.

In an aspect of a disclosed pharmaceutical formulation, a therapeutically effective amount of SBI-810 can comprise a dose of about 1 mg/kg/day to about 100 mg/kg/day. In an aspect of a disclosed pharmaceutical formulation, a therapeutically effective amount of SBI-810 can comprise a dose of about 1 mg/kg/day to about 20 mg/kg/day, about 20 mg/kg/day to about 40 mg/kg/day, about 40 mg/kg/day to about 60 mg/kg/day, about 60 mg/kg/day to about 80 mg/kg/day, or about 80 mg/kg/day to about 100 mg/kg/day. In an aspect of a disclosed pharmaceutical formulation, a therapeutically effective amount of SBI-810 can comprise a dose of about 1 mg/kg/day, about 5 mg/kg/day, about 10 mg/kg/day, about 15 mg/kg/day, about 20 mg/kg/day, about 25 mg/kg/day, about 30 mg/kg/day, about 35 mg/kg/day, about 40 mg/kg/day, about 45 mg/kg/day, about 50 mg/kg/day, about 55 mg/kg/day, about 60 mg/kg/day, about 65 mg/kg/day, about 70 mg/kg/day, about 75 mg/kg/day, about 80 mg/kg/day, about 85 mg/kg/day, about 90 mg/kg/day, about 95 mg/kg/day, or about 100 mg/kg/day. In an aspect of a disclosed pharmaceutical formulation, a therapeutically effective amount of SBI-810 can comprise a dose of more than 100 mg/kg/day.

In an aspect, a disclosed pharmaceutical formulation comprising a therapeutically effective amount of SBI-810 can further comprise a therapeutically effective amount of one or more therapeutic agents. A disclosed therapeutic agent can be any disclosed agent that effects a desired outcome (e.g., biologically active agents, pharmaceutically active agents, chemotherapeutic agents, anti-bacterial agents, anti-fungal agents, anti-viral agents, corticosteroids, analgesics, immunostimulants, immune-based products, or any combination thereof) and are disclosed supra.

In an aspect, a disclosed pharmaceutical formulation comprising a therapeutically effective amount of SBI-810 can further comprise a STING agonist, a steroid, an opioid analgesic, a local anesthetic, an NSAID, a programmed cell death 1 ligand, an anti-cancer agent, and any combination thereof. In an aspect, a disclosed opioid analgesic can comprise codeine, dextropropyoxyphene, diamorphine, dihydrocodeine, meptazinol, methadone, morphine, nalbuphine, pentazocine, oxycodone, and any combination thereof.

In an aspect, a disclosed pharmaceutical formulation comprising a therapeutically effective amount of SBI-810 can treat and/or prevent pain in a subject. In an aspect, a disclosed pharmaceutical formulation comprising a therapeutically effective amount of SBI-810 can cause a subject to experience less pain, less intense pain, a shorter duration of pain, or a combination thereof.

In an aspect, a disclosed pharmaceutical formulation comprising a therapeutically effective amount of SBI-810 can improve a subject's ability to move and/or level of mobility. In an aspect, a disclosed pharmaceutical formulation comprising a therapeutically effective amount of SBI-810can improve a subject's ability to move and/or level of mobility by about 10%, about 20%, about 30%, about 40%, about 50%, about 60%, about 70%, about 80%, about 90%, or about 100% as compared to native or control levels or to a subject's level of mobility prior to the administration of the disclosed pharmaceutical formulation comprising a therapeutically effective amount of SBI-810. In an aspect, a disclosed pharmaceutical formulation comprising a therapeutically effective amount of SBI-810 can improve a subject's ability to move and/or level of mobility by about 10-20%, about 20-30%, about 30-40%, about 40-50%, about 50-60%, about 60-70%, about 70-80%, about 80-90%, or about 90-100% as compared to native or control levels or to a subject's ability to move and/or level of mobility prior to the administration of the disclosed pharmaceutical formulation comprising a therapeutically effective amount of SBI-810. In an aspect, a disclosed pharmaceutical formulation comprising a therapeutically effective amount of SBI-810 can improve a subject's ability to move and/or level of mobility by about 0-25%, about 25-50%, about 50-75%, or about 75-100 as compared to native or control levels or to a subject's ability to move and/or level of mobility prior to the administration of the disclosed pharmaceutical formulation comprising a therapeutically effective amount of SBI-810. In an aspect, a subject's ability to move and/or level of mobility prior to the administration of a disclosed pharmaceutical formulation comprising a therapeutically effective amount of SBI-810 can be used as a control.

In an aspect, a disclosed pharmaceutical formulation comprising a therapeutically effective amount of SBI-810 does not elicit hypothermia, hypotension, and/or motor impairment in a subject.

In an aspect, a disclosed pharmaceutical formulation comprising a therapeutically effective amount of SBI-810 can be prepared for systemic or direct administration. In an aspect, a disclosed pharmaceutical formulation comprising a therapeutically effective amount of SBI-810 can be prepared for any disclosed method of administration. In an aspect, a disclosed pharmaceutical formulation comprising a therapeutically effective amount of SBI-810 can be prepared for oral administration, intrathecal administration, intravenous administration, intra-articular administration, epidural administration, intratumoral administration, i intraperitoneal administration, or any combination thereof. In an aspect, a disclosed pharmaceutical formulation comprising a therapeutically effective amount of SBI-810 can be prepared for any method of administration disclosed herein. In an aspect, a disclosed pharmaceutical formulation comprising a therapeutically effective amount of SBI-810 can be prepared for administration via multiple routes either concurrently or sequentially. For example, in an aspect, a disclosed pharmaceutical formulation comprising a therapeutically effective amount of SBI-810 can be first administered orally and then be administered intravenously. In an aspect, a disclosed pharmaceutical formulation comprising a therapeutically effective amount of SBI-810 can be first administered intravenously and then be administered orally. A skilled clinical can determine the best route of administration for a subject at a given time.

In an aspect, disclosed pharmaceutical formulation comprising a therapeutically effective amount of SBI-810 can be formulated for oral administration, intravenous administration, intrathecal administration, intra-articular administration, epidural administration, intraperitoneal administration, or any combination thereof. In an aspect, a disclosed pharmaceutical formulation comprising a therapeutically effective amount of SBI-810 can be formulated for administration via topical administration (e.g., skin patch) or depot administration. In an aspect, disclosed pharmaceutical formulation comprising a therapeutically effective amount of SBI-810 can be formulated for any route of administration disclosed herein.

In an aspect, a disclosed pharmaceutical formulation comprising a therapeutically effective amount of SBI-810 can restore one or more aspects of cellular homeostasis and/or cellular functionality and/or metabolic dysregulation in a subject, such as, for example, a subject having acute and/or chronic pain. In an aspect, restoring one or more aspects of cellular homeostasis and/or cellular functionality can comprise reducing the subject's acute and/or chronic pain.

In an aspect, a disclosed pharmaceutical formulation comprising a therapeutically effective amount of SBI-810 can reduce a subject's pain by about 10%, about 20%, about 30%, about 40%. about 50%, about 60%, about 70%, about 80%, about 90%, or about 100% as compared to native or control levels or to the subject's level prior to the onset of pain (e.g., acute and/or chronic pain due to trauma, injury, cancer, disease and/or disorder, neuropathic pain, etc.). In an aspect, a disclosed pharmaceutical formulation comprising a therapeutically effective amount of SBI-810 can reduce a subject's pain by about 10-20%, about 20-30%, about 30-40%, about 40-50%, about 50-60%, about 60-70%, about 70-80%, about 80-90%, or about 90-100% as compared to native or control levels or to a subject's level prior to the onset of pain (e.g., acute and/or chronic pain due to trauma, injury, cancer, disease and/or disorder, neuropathic pain, etc.). In an aspect, a disclosed pharmaceutical formulation comprising a therapeutically effective amount of SBI-810 can reduce a subject's pain by about 0)-25%, about 25-50%, about 50-75%, or about 75-100% as compared to native or control levels or to a subject's pain level prior to the onset of pain (e.g., acute and/or chronic pain due to trauma, injury, cancer, disease and/or disorder, neuropathic pain. etc.). In an aspect, a subject's pain level prior to the onset of pain can be used as a control.

In an aspect, a subject's acute and/or chronic pain can comprise back pain (including upper back pain and/or lower back pain), cancer pain, arthritis pain, headaches (including but not limited to migraines, cluster headaches, and tension headaches), neuralgias, neuropathies, pain due to a CNS injury, pain due to a PNS injury, pain with no known etiological cause, or any combination thereof. In an aspect, a subject's pain can be cancer pain. In an aspect, a subject's pain can be arthritis pain. In an aspect, a subject's pain can be headache pain. In an aspect, a subject's pain can be neuralgia pain. In an aspect, a subject's pain can be neuropathy pain. In an aspect, a subject's pain can be due to an injured sustained while serving in the armed forces or military.

Disclosed herein is a pharmaceutical formulation comprising a therapeutically effective amount of SBI-810, wherein the composition can cause the subject to experience less pain, less intense pain, a shorter duration of pain, or a combination thereof. Disclosed herein is a pharmaceutical formulation comprising a therapeutically effective amount of SBI-810, wherein the composition can improve the subject's ability to move and/or level of mobility. Disclosed herein is a pharmaceutical formulation comprising a therapeutically effective amount of SBI-810, wherein the composition does not elicit hypothermia, hypotension, and/or motor impairment in a subject. Disclosed herein is a pharmaceutical formulation comprising a therapeutically effective amount of SBI-810, wherein the composition does not elicit hypothermia, hypotension, and/or motor impairment in a subject, wherein the subject is in the military or is veteran.

E. Kits

Disclosed herein is a kit comprising a disclosed composition comprising a therapeutically effective amount of a NTSR1 ligand, a disclosed pharmaceutical formulation comprising a therapeutically effective amount of a NTSR1 ligand, or any combination thereof. Disclosed herein is a kit comprising a disclosed composition comprising a therapeutically effective amount of SBI-553, a disclosed pharmaceutical formulation comprising a therapeutically effective amount of SBI-553, or any combination thereof. Disclosed herein is a kit comprising a disclosed composition comprising a therapeutically effective amount of SBI-810, a disclosed pharmaceutical formulation comprising a therapeutically effective amount of SBI-810, or any combination thereof. Disclosed herein is a kit comprising a disclosed composition comprising a therapeutically effective amount of a NTSR1 ligand, a disclosed pharmaceutical formulation comprising a therapeutically effective amount of a NTSR1 ligand, or any combination thereof, and one or more therapeutic agents. Disclosed herein is a kit comprising a disclosed composition comprising a therapeutically effective amount of SBI-553, a disclosed pharmaceutical formulation comprising a therapeutically effective amount of SBI-553, or any combination thereof, and one or more therapeutic agents. Disclosed herein is a kit comprising a disclosed composition comprising a therapeutically effective amount of SBI-810, a disclosed pharmaceutical formulation comprising a therapeutically effective amount of SBI-810, or any combination thereof, and one or more therapeutic agents.

In an aspect, a disclosed kit can comprise one or more therapeutic agents. “Agents” and “Therapeutic Agents” are known to the art and are described supra. In an aspect, a disclosed therapeutic agent can treat, prevent, inhibit, and/or ameliorate one or more comorbidities in a subject.

In an aspect, a disclosed kit can comprise at least two components constituting the kit. Together, the components constitute a functional unit for a given purpose (such as, for example, treating a subject diagnosed with or suspected of having acute pain and/or chronic pain). Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation. In an aspect, a kit for use in a disclosed method can comprise one or more containers holding a disclosed composition, a disclosed pharmaceutical formulation, a disclosed therapeutic agent, and a label or package insert with instructions for use. In an aspect, suitable containers include, for example, bottles, vials, syringes, blister pack, etc. The containers can be formed from a variety of materials such as glass or plastic. The container can hold a disclosed composition, a disclosed pharmaceutical formulation, a disclosed therapeutic agent, or a combination thereof, and can have a sterile access port (for example the container may be an intravenous solution bag or a vial having a stopper pierceable by a hypodermic injection needle). The label or package insert can indicate that a disclosed composition, a disclosed pharmaceutical formulation, a disclosed therapeutic agent, or a combination thereof, can be used for treating, preventing, inhibiting, and/or ameliorating acute pain and/or chronic pain or a disease or disorder or complications and/or symptoms associated with a disease or disorder that inflicts pain on a subject. A kit can comprise additional components necessary for administration such as, for example, other buffers, diluents, filters, needles, and syringes. In an aspect, a disclosed kit can be used to treat and/or prevent acute pain, chronic pain, or both.

F. Methods of Treating and/or Preventing Pain

Disclosed herein is a method of treating and/or preventing pain in a subject, the method comprising administering to a subject in need thereof a therapeutically effective amount of a disclosed composition or a disclosed pharmaceutical formulation, wherein pain is alleviated and/or minimized in the subject.

### 1. Methods Using NTSR1 Ligands

Disclosed herein is a method of treating and/or preventing pain in a subject, the method comprising administering to a subject in need thereof a composition or a pharmaceutical formulation comprising a therapeutically effective amount of a NTSR1 ligand or a pharmaceutically acceptable salt, solvate, prodrug, or N-oxide thereof, wherein pain is alleviated and/or minimized in the subject.

In an aspect of a disclosed method, a disclosed NTSR1 ligand can comprise a functionally selective β-arrestin biased ligand.

In an aspect of a disclosed method, a disclosed functionally selective β-arrestin biased ligand can comprise the general formula (I):

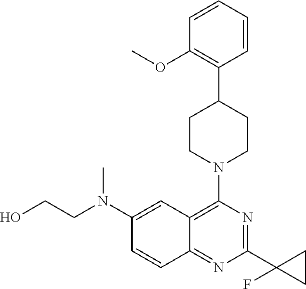

or a pharmaceutically acceptable salt, solvate, hydrate, prodrug, ester, or derivative thereof. In an aspect of a disclosed method, a disclosed functionally selective β-arrestin biased ligand can comprise SBI-553.

In an aspect of a disclosed method, a disclosed functionally selective β-arrestin biased ligand can comprise the general formula (II):

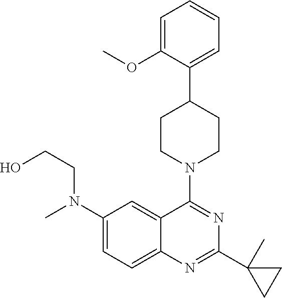

or a pharmaceutically acceptable salt, solvate, hydrate, prodrug, ester, or derivative thereof. In an aspect of a disclosed method, a disclosed functionally selective β-arrestin biased ligand can comprise SBI-810.

In an aspect of a disclosed method, a therapeutically effective amount of the NTSR1 ligand can comprise a dose of about 1 mg/kg/day to about 100 mg/kg/day. In an aspect of a disclosed method, a therapeutically effective amount of the NTSR1 ligand can comprise a dose of about 1 mg/kg/day to about 20 mg/kg/day, about 20 mg/kg/day to about 40 mg/kg/day, about 40 mg/kg/day to about 60 mg/kg/day, about 60 mg/kg/day to about 80 mg/kg/day, or about 80 mg/kg/day to about 100 mg/kg/day. In an aspect of a disclosed method, a therapeutically effective amount of the NTSR1 ligand can comprise a dose of about 1 mg/kg/day, about 5 mg/kg/day, about 10 mg/kg/day, about 15 mg/kg/day, about 20 mg/kg/day, about 25 mg/kg/day, about 30 mg/kg/day, about 35 mg/kg/day, about 40 mg/kg/day, about 45 mg/kg/day, about 50 mg/kg/day, about 55 mg/kg/day, about 60 mg/kg/day, about 65 mg/kg/day, about 70 mg/kg/day, about 75 mg/kg/day, about 80 mg/kg/day, about 85 mg/kg/day, about 90 mg/kg/day, about 95 mg/kg/day, or about 100 mg/kg/day. In an aspect of a disclosed method, a therapeutically effective amount of the NTSR1 ligand can comprise a dose of more than 100 mg/kg/day.

In an aspect of a disclosed method, a disclosed composition or a disclosed pharmaceutical formulation comprising a therapeutically effective amount of a NTSR1 ligand can further comprise a therapeutically effective amount of one or more therapeutic agents. A disclosed therapeutic agent can be any disclosed agent that effects a desired clinical outcome (e.g., biologically active agents, pharmaceutically active agents, chemotherapeutic agents, anti-bacterial agents, anti-fungal agents, anti-viral agents, corticosteroids, analgesics, immunostimulants, immune-based products, or any combination thereof) and are disclosed supra.

In an aspect of a disclosed method, one or more therapeutic agents can comprise a STING agonist, a steroid, an opioid analgesic, a local anesthetic, an NSAID, a programmed cell death 1 ligand, an anti-cancer agent, and any combination thereof. In an aspect of a disclosed method, a disclosed opioid analgesic can comprise codeine, dextropropyoxyphene, diamorphine, dihydrocodeine, meptazinol, methadone, morphine, nalbuphine, pentazocine, oxycodone, and any combination thereof.

In an aspect, a disclosed method can treat and/or prevent pain in a subject. In an aspect, a disclosed method can cause a subject to experience less pain, less intense pain, a shorter duration of pain, or a combination thereof.

In an aspect, a disclosed method does not elicit hypothermia, hypotension, and/or motor impairment in a subject.

In an aspect, a disclosed method of treating and/or preventing pain in a subject can further comprise repeating the administering of a disclosed composition or a disclosed pharmaceutical formulation comprising a therapeutically effective amount of a NTSR1 ligand to the subject.

In an aspect of a disclosed method, administering a disclosed composition or a disclosed formulation comprising a therapeutically effective amount of a NTSR1 ligand can comprise systemic or direct administration. In an aspect, administering a disclosed composition or a disclosed pharmaceutical formulation comprising a therapeutically effective amount of a NTSR1 ligand can comprise oral administration, intravenous administration, intra-articular administration, epidural administration, intrathecal administration, intraperitoneal administration, topical administration (e.g., skin patch), depot administration, or any combination thereof. In an aspect, administering a disclosed composition or a disclosed pharmaceutical formulation comprising a therapeutically effective amount of a NTSR1 ligand can be administered by any method of administration disclosed herein. In an aspect, a disclosed composition or a disclosed pharmaceutical formulation comprising a therapeutically effective amount of a NTSR1 ligand can be administered via multiple routes either concurrently or sequentially. For example, in an aspect. a disclosed composition or a disclosed pharmaceutical formulation can be first administered orally and then be administered intravenously. In an aspect, administering a disclosed composition or a disclosed pharmaceutical formulation comprising a therapeutically effective amount of a NTSR1 ligand can be first administered intravenously and then be administered orally. A skilled clinician can determine the best route of administration for a subject at a given time.

In an aspect of a disclosed method, administering a disclosed composition or a disclosed pharmaceutical formulation comprising a therapeutically effective amount of a NTSR1 ligand and/or a disclosed therapeutic agent can comprise continuous administration, non-continuous administration, or intermittent administration. In an aspect, continuous administration can comprise the use of an infusion pump.

In an aspect, a subject can be in the military or can be a veteran. In an aspect, a subject can have acute pain, chronic pain, or both.

In an aspect, a subject's acute and/or chronic pain can comprise back pain (including upper back pain and/or lower back pain), cancer pain, arthritis pain, headaches (including but not limited to migraines, cluster headaches, and tension headaches), neuralgias, neuropathies, pain due to a CNS injury, pain due to a PNS injury, pain with no known etiological cause, or any combination thereof.

In an aspect, a subject can have one or more bone fractures. In an aspect, the one or more bone fractures can be the result of a non-trauma, a disease, or a disorder. In an aspect, a subject can be diagnosed with or can be suspected of having a bone fracture or more than one bone fractures. In an aspect the subject can have multiple fractures in the same bone, multiple fractures in different bones, or both. In an aspect of a disclosed method, a fracture can comprise an open fracture, a closed fracture, a partial fracture, a complete fracture, a stable fracture, or a displaced fracture. In an aspect of a disclosed method, a fracture comprises a transverse fracture, a spiral fracture, a greenstick fracture, a stress fracture, a compression fracture, an oblique fracture, an impacted fracture, a segmental fracture, a comminuted fracture, or an avulsion fracture. In an aspect, a subject can have one or more types of fractures. In an aspect, a disclosed method can further comprise identifying the bone or bones having a fracture or fractures. In an aspect, a subject can have non-fracture pain.

In an aspect, a subject can have sustained one or more injuries and/or one or more physical injuries. In an aspect, a subject can have experienced one or more surgical procedures. In an aspect, a subject can have opioid dependence or can have recovered from an opioid dependence.

In an aspect, a subject can have been diagnosed with cancer. In an aspect, cancer can comprise ovarian cancer, ovarian adenocarcinoma, ovarian teratocarcinoma, lung cancer, small cell lung cancer (SCLC), non-small cell lung cancer (NSCLC), squamous cell lung carcinoma, adenocarcinoma, gastric cancer, breast cancer, hepatic cancer, pancreatic cancer, skin cancer, in particular basal cell carcinoma and squamous cell carcinoma, malignant melanoma, head and neck cancer, malignant pleomorphic adenoma, sarcoma, synovial sarcoma, carcinosarcoma, bile duct cancer, bladder cancer, transitional cell carcinoma, papillary carcinoma, kidney cancer, renal cell carcinoma, clear cell renal cell carcinoma, papillary renal cell carcinoma, colon cancer, small bowel cancer, small bowel adenocarcinoma, adenocarcinoma of the ileum, testicular embryonal carcinoma, placental choriocarcinoma, cervical cancer, testicular cancer, testicular seminoma, testicular teratoma, embryonic testicular cancer, uterine cancer, teratocarcinoma, embryonal carcinoma, or any combination thereof.

In an aspect, a subject can be a non-human mammal or a human. A subject can be of any age, such as, for example, a geriatric, an adult, an adolescent, a child, or a baby.

In an aspect, a disclosed method can further comprise assessing the subject's pain. The subject's pain can be assessed subjectively or objectively as known to the art.

In an aspect of a disclosed method, following the administering step, the subject can experience less pain, less intense pain, a shorter duration of pain, or a combination thereof.

In an aspect, a subject's pain can be treated and/or prevented by diminishing the intensity, the duration, the amount, or a combination thereof of a subject's pain (e.g., acute and/or chronic pain). In an aspect, a disclosed method can reduce a subject's pain by about 10%, about 20%, about 30%, about 40%, about 50%, about 60%, about 70%, about 80%, about 90%, or about 100% as compared to native or control levels or to a subject's level prior to the onset of pain (e.g., acute and/or chronic pain due to trauma, injury, cancer, disease and/or disorder, neuropathic pain, etc.). In an aspect, a disclosed method can reduce a subject's pain by about 10-20%, about 20-30%, about 30-40%, about 40-50%, about 50-60%, about 60-70%, about 70-80%, about 80-90%, or about 90-100% as compared to native or control levels or to a subject's level prior to the onset of pain (e.g., acute and/or chronic pain due to trauma, injury, cancer, disease and/or disorder, neuropathic pain, etc.). In an aspect, a disclosed method can reduce a subject's pain by about 0-25%, about 25-50%, about 50-75%, or about 75-100% as compared to native or control levels or to a subject's pain level prior to the onset of pain (e.g., acute and/or chronic pain due to trauma, injury, cancer, disease and/or disorder, neuropathic pain, etc.). In an aspect, a subject's pain level prior to the onset of pain can be used as a control.

In an aspect, a subject's acute and/or chronic pain can comprise back pain (including upper back pain and/or lower back pain), cancer pain, arthritis pain, headaches (including but not limited to migraines, cluster headaches, and tension headaches), neuralgias, neuropathies, pain due to a CNS injury, pain due to a PNS injury, pain with no known etiological cause, or any combination thereof. In an aspect, a subject's pain can be cancer pain. In an aspect, a subject's pain can be arthritis pain. In an aspect, a subject's pain can be headache pain. In an aspect, a subject's pain can be neuralgia pain. In an aspect, a subject's pain can be neuropathy pain. In an aspect, a subject's pain can be due to an injured sustained while serving in the armed forces or military.

In an aspect, a disclosed method can further comprise improving a subject's ability to move and/or level of mobility. Any improvement in a subject's ability to move and/or level of mobility can be assessed subjectively or objectively as known to the art.

In an aspect, a disclosed method can improve a subject's ability to move and/or level of mobility by about 10%, about 20%, about 30%, about 40%, about 50%, about 60%, about 70%, about 80%, about 90%, or about 100% as compared to native or control levels or to a subject's ability to move and/or level of mobility prior to the administration of a disclosed composition or a disclosed pharmaceutical formulation comprising a therapeutically effective amount of a NTSR1 ligand. In an aspect, a disclosed method can improve a subject's ability to move and/or level of mobility by about 10-20%, about 20-30%, about 30-40%, about 40-50%, about 50-60%, about 60-70%, about 70-80%, about 80-90%, or about 90-100% as compared to native or control levels or to a subject's ability to move and/or level of mobility prior to the administration of a disclosed composition or a disclosed pharmaceutical formulation comprising a therapeutically effective amount of a NTSR1 ligand. In an aspect, a disclosed method can improve a subject's ability to move and/or level of mobility by about 0-25%, about 25-50%, about 50-75%, or about 75-100 as compared to native or control levels or to a subject's ability to move and/or level of mobility prior to the administration of a disclosed composition or a disclosed pharmaceutical formulation comprising a therapeutically effective amount of a NTSR1 ligand. In an aspect, a subject's ability to move and/or level of mobility prior to the administration of a disclosed composition or a disclosed pharmaceutical formulation can be used as a control.

In an aspect, a disclosed method can further comprise administering to the subject a therapeutically effective amount of one or more therapeutic agents. A disclosed therapeutic agent can be any disclosed agent that effects a desired clinical outcome (e.g., biologically active agents, pharmaceutically active agents, chemotherapeutic agents, anti-bacterial agents, anti-fungal agents, anti-viral agents, corticosteroids, analgesics, immunostimulants, immune-based products, or any combination thereof) and are disclosed supra.

In an aspect, the one or more therapeutic agents can be administered to the subject prior to, concurrently, or after the administration of a disclosed composition or a disclosed pharmaceutical formulation comprising a therapeutically effective amount of a NTSR1 ligand.

In an aspect, a disclosed method can further comprise repeating the administering of the one or more therapeutic agents to the subject.

In an aspect, a disclosed method can further comprise monitoring the subject for adverse effects. In an aspect, in the absence of adverse effects, a disclosed method can comprise continuing to administer a disclosed composition or a disclosed pharmaceutical formulation comprising a therapeutically effective amount of a NTSR1 ligand. In an aspect, in the presence of adverse effects, a disclosed method can comprise modifying one or more steps of a disclosed method. In an aspect, a disclosed method can further comprise modifying an aspect of the method. In an aspect, modifying an aspect of a disclosed method can comprise changing the amount of a disclosed composition or a disclosed pharmaceutical formulation comprising a therapeutically effective amount of a NTSR1 ligand and/or a disclosed therapeutic agent administered to the subject, changing the frequency of administration of a disclosed composition or a disclosed pharmaceutical formulation comprising a therapeutically effective amount of a NTSR1 ligand and/or a disclosed therapeutic agent to the subject, changing the duration of time that disclosed composition or a disclosed pharmaceutical formulation comprising a therapeutically effective amount of a NTSR1 ligand and/or a disclosed therapeutic agent is administered to a subject, or any combination thereof.

In an aspect, a disclosed method does not elicit hypothermia, hypotension, and/or motor impairment in the subject.

In an aspect, a disclosed method can further comprise administering to the subject one or more additional anti-cancer therapies. Anti-cancer therapies are known to the art. In an aspect, a disclosed anti-cancer therapy can comprise endocrine therapy, radiotherapy, hormone therapy, gene therapy, thermal therapy, ultrasound therapy, or any combination thereof. In an aspect, a disclosed anti-cancer therapy can comprise one or more chemotherapeutic agents. In an aspect, a disclosed chemotherapeutic agent can comprise an anthracycline, a vinca alkaloid, an alkylating agent, an immune cell antibody, an antimetabolite, a TNFR glucocorticoid induced TNFR related protein (GITR) agonist, a proteasome inhibitor, an immunomodulator, or any combination thereof.

In an aspect, a disclosed method comprising a therapeutically effective amount of a NTSR1 ligand can restore one or more aspects of cellular homeostasis and/or cellular functionality and/or metabolic dysregulation in a subject, such as, for example, a subject having acute and/or chronic pain. In an aspect, restoring one or more aspects of cellular homeostasis and/or cellular functionality can comprise reducing the subject's acute and/or chronic pain.

### 2. Methods Using SBI-553

Disclosed herein is a method of treating and/or preventing pain in a subject, the method comprising administering to a subject in need thereof a composition comprising a therapeutically effective amount of SBI-553, wherein pain is alleviated and/or minimized in the subject. Disclosed herein is a method of treating and/or preventing pain in a subject, the method comprising administering to a subject in need thereof a pharmaceutical formulation comprising a therapeutically effective amount of SBI-553, wherein pain is alleviated and/or minimized in the subject.

In an aspect of a disclosed method, a therapeutically effective amount of SBI-553 can comprise a dose of about 1 mg/kg/day to about 100 mg/kg/day. In an aspect of a disclosed method, a therapeutically effective amount of SBI-553 can comprise a dose of about 1 mg/kg/day to about 20 mg/kg/day, about 20 mg/kg/day to about 40 mg/kg/day, about 40 mg/kg/day to about 60 mg/kg/day, about 60 mg/kg/day to about 80 mg/kg/day, or about 80 mg/kg/day to about 100 mg/kg/day. In an aspect of a disclosed method, a therapeutically effective amount of SBI-553 can comprise a dose of about 1 mg/kg/day, about 5 mg/kg/day, about 10 mg/kg/day, about 15 mg/kg/day, about 20 mg/kg/day, about 25 mg/kg/day, about 30 mg/kg/day, about 35 mg/kg/day, about 40 mg/kg/day, about 45 mg/kg/day, about 50 mg/kg/day, about 55 mg/kg/day, about 60 mg/kg/day, about 65 mg/kg/day, about 70 mg/kg/day, about 75 mg/kg/day, about 80 mg/kg/day, about 85 mg/kg/day, about 90 mg/kg/day, about 95 mg/kg/day, or about 100 mg/kg/day. In an aspect of a disclosed method, a therapeutically effective amount of SBI-553 can comprise a dose of more than 100 mg/kg/day.

In an aspect of a disclosed method, a disclosed composition or a disclosed pharmaceutical formulation comprising a therapeutically effective amount of SBI-553 can further comprise a therapeutically effective amount of one or more therapeutic agents. A disclosed therapeutic agent can be any disclosed agent that effects a desired clinical outcome (e.g., biologically active agents, pharmaceutically active agents, chemotherapeutic agents, anti-bacterial agents, anti-fungal agents, anti-viral agents, corticosteroids, analgesics, immunostimulants, immune-based products, or any combination thereof) and are disclosed supra.

In an aspect of a disclosed method, one or more therapeutic agents can comprise a therapeutically effective amount of a STING agonist, a steroid, an opioid analgesic, a local anesthetic, an NSAID, a programmed cell death 1 ligand, an anti-cancer agent, and any combination thereof.

In an aspect of a disclosed method, a disclosed opioid analgesic can comprise codeine, dextropropyoxyphene, diamorphine, dihydrocodeine, meptazinol, methadone, morphine, nalbuphine, pentazocine, oxycodone, and any combination thereof.

In an aspect, a disclosed method can treat and/or prevent pain in a subject.

In an aspect, a disclosed method can cause a subject to experience less pain, less intense pain, a shorter duration of pain, or a combination thereof.

In an aspect, a disclosed method can improve a subject's ability to move and/or level of mobility.

In an aspect, a disclosed method can improve a subject's ability to move and/or level of mobility by about 10%, about 20%, about 30%, about 40%, about 50%, about 60%, about 70%, about 80%, about 90%, or about 100% as compared to native or control levels or to the subject's level of mobility prior to the administration of a disclosed composition or a disclosed pharmaceutical formulation comprising a therapeutically effective amount of SBI-553. In an aspect, a disclosed method can improve a subject's ability to move and/or level of mobility by about 10-20%, about 20-30%, about 30-40%, about 40-50%, about 50-60%, about 60-70%, about 70-80%, about 80-90%, or about 90-100% as compared to native or control levels or to a subject's ability to move and/or level of mobility prior to the administration of a disclosed composition or a disclosed pharmaceutical formulation comprising a therapeutically effective amount of SBI-553. In an aspect, a disclosed method can improve a subject's ability to move and/or level of mobility by about 0)-25%, about 25-50%, about 50-75%, or about 75-100 as compared to native or control levels or to a subject's ability to move and/or level of mobility prior to the administration of a disclosed composition or a disclosed pharmaceutical formulation comprising SBI-553. In an aspect, a subject's ability to move and/or level of mobility prior to the administration of the disclosed composition or a disclosed pharmaceutical formulation comprising a therapeutically effective amount of SBI-553 can be used as a control.

In an aspect, a disclosed method does not elicit hypothermia, hypotension, and/or motor impairment in a subject.

In an aspect, a disclosed method of treating and/or preventing pain in a subject can further comprise repeating the administering of a disclosed composition or a disclosed pharmaceutical formulation comprising a therapeutically effective amount of SBI-553 to the subject.

In an aspect of a disclosed method, administering a disclosed composition or a disclosed formulation comprising SBI-553 can comprise systemic or direct administration. In an aspect, administering a disclosed composition or a disclosed pharmaceutical formulation comprising SBI-553 can comprise oral administration, intravenous administration, intra-articular administration, epidural administration, intrathecal administration, topical administration (e.g., skin patch), depot administration, intraperitoneal administration, or any combination thereof. In an aspect, administering a disclosed composition or a disclosed pharmaceutical formulation comprising SBI-553 can be administered by any method of administration disclosed herein. In an aspect, a disclosed composition or a disclosed pharmaceutical formulation comprising SBI-553 can be administered via multiple routes either concurrently or sequentially. For example, in an aspect, a disclosed composition or a disclosed pharmaceutical formulation comprising SBI-553 can be first administered orally and then be administered intravenously. In an aspect, administering a disclosed composition or a disclosed pharmaceutical formulation comprising SBI-553 can be first administered intravenously and then be administered orally. A skilled clinician can determine the best route of administration for a subject at a given time.

In an aspect of a disclosed method, administering a disclosed composition or a disclosed pharmaceutical formulation comprising SBI-553 and/or a disclosed therapeutic agent can comprise continuous administration, non-continuous administration, or intermittent administration. In an aspect, continuous administration can comprise the use of an infusion pump.

In an aspect, a subject can be in the military or can be a veteran. In an aspect, a subject can have acute pain, chronic pain, or both. In an aspect, a subject's acute and/or chronic pain can comprise back pain (including upper back pain and/or lower back pain), cancer pain, arthritis pain, headaches (including but not limited to migraines, cluster headaches, and tension headaches), neuralgias, neuropathies, pain due to a CNS injury, pain due to a PNS injury, pain with no known etiological cause, or any combination thereof.

In an aspect, a subject can have one or more bone fractures. In an aspect, the one or more bone fractures can be the result of a non-trauma, a disease, or a disorder. In an aspect, a subject can be diagnosed with or can be suspected of having a bone fracture or more than one bone fractures. In an aspect the subject can have multiple fractures in the same bone, multiple fractures in different bones, or both. In an aspect of a disclosed method, a fracture can comprise an open fracture, a closed fracture, a partial fracture, a complete fracture, a stable fracture, or a displaced fracture. In an aspect of a disclosed method, a fracture comprises a transverse fracture, a spiral fracture, a greenstick fracture, a stress fracture, a compression fracture, an oblique fracture, an impacted fracture, a segmental fracture, a comminuted fracture, or an avulsion fracture. In an aspect, a subject can have one or more types of fractures. In an aspect, a disclosed method can comprise identifying the bone or bones having a fracture or fractures. In an aspect, a subject can have non-fracture pain.

In an aspect, a subject can have sustained one or more injuries and/or one or more physical injuries. In an aspect, a subject can have experienced one or more surgical procedures. In an aspect, a subject can have opioid dependence or can have recovered from an opioid dependence.

In an aspect, a subject can have been diagnosed with cancer. In an aspect, cancer can comprise ovarian cancer, ovarian adenocarcinoma, ovarian teratocarcinoma, lung cancer, small cell lung cancer (SCLC), non-small cell lung cancer (NSCLC), squamous cell lung carcinoma, adenocarcinoma, gastric cancer, breast cancer, hepatic cancer, pancreatic cancer, skin cancer, in particular basal cell carcinoma and squamous cell carcinoma, malignant melanoma, head and neck cancer, malignant pleomorphic adenoma, sarcoma, synovial sarcoma, carcinosarcoma, bile duct cancer, bladder cancer, transitional cell carcinoma, papillary carcinoma, kidney cancer, renal cell carcinoma, clear cell renal cell carcinoma, papillary renal cell carcinoma, colon cancer, small bowel cancer, small bowel adenocarcinoma, adenocarcinoma of the ileum, testicular embryonal carcinoma, placental choriocarcinoma, cervical cancer, testicular cancer, testicular seminoma, testicular teratoma, embryonic testicular cancer, uterine cancer, teratocarcinoma, embryonal carcinoma, or any combination thereof.

In an aspect, a subject can be a non-human mammal or a human. A subject can be of any age, such as, for example, a geriatric, an adult, an adolescent, a child, or a baby.

In an aspect, a disclosed method can further comprise assessing the subject's pain. The subject's pain can be assessed subjectively or objectively as known to the art. In an aspect of a disclosed method, following the administering step, the subject can experience less pain, less intense pain, a shorter duration of pain, or a combination thereof.

In an aspect, a subject's pain can be treated and/or prevented by diminishing the intensity, the duration, the amount, or a combination thereof of a subject's pain (e.g., acute and/or chronic pain). In an aspect, a disclosed method can reduce a subject's pain by about 10%, about 20%, about 30%, about 40%, about 50%, about 60%, about 70%, about 80%, about 90%, or about 100% as compared to native or control levels or to a subject's level prior to the onset of pain (e.g., acute and/or chronic pain due to trauma, injury, cancer, disease and/or disorder, neuropathic pain, etc.). In an aspect, a disclosed method can reduce a subject's pain by about 10-20%, about 20-30%, about 30-40%, about 40-50%, about 50-60%, about 60-70%, about 70-80%, about 80-90%, or about 90-100% as compared to native or control levels or to a subject's level prior to the onset of pain (e.g., acute and/or chronic pain due to trauma, injury, cancer, disease and/or disorder, neuropathic pain, etc.). In an aspect, a disclosed method can reduce a subject's pain by about 0-25%, about 25-50%, about 50-75%, or about 75-100% as compared to native or control levels or to a subject's pain level prior to the onset of pain (e.g., acute and/or chronic pain due to trauma, injury, cancer, disease and/or disorder, neuropathic pain, etc.). In an aspect, a subject's pain level prior to the onset of pain can be used as a control.

In an aspect, a subject's acute and/or chronic pain can comprise back pain (including upper back pain and/or lower back pain), cancer pain, arthritis pain, headaches (including but not limited to migraines, cluster headaches, and tension headaches), neuralgias, neuropathies, pain due to a CNS injury, pain due to a PNS injury, pain with no known etiological cause, or any combination thereof. In an aspect, a subject's pain can be cancer pain. In an aspect, a subject's pain can be arthritis pain. In an aspect, a subject's pain can be headache pain. In an aspect, a subject's pain can be neuralgia pain. In an aspect, a subject's pain can be neuropathy pain. In an aspect, a subject's pain can be due to an injured sustained while serving in the armed forces or military.

In an aspect, a disclosed method can further comprise improving a subject's ability to move and/or level of mobility. Any improvement in the subject's ability to move and/or level of mobility can be assessed subjectively or objectively as known to the art. In an aspect, a disclosed method can improve the subject's ability to move and/or level of mobility by about 10%, about 20%, about 30%, about 40%, about 50%, about 60%, about 70%, about 80%, about 90%, or about 100% as compared to native or control levels or to the subject's ability to move and/or level of mobility prior to the administration of a disclosed composition or a disclosed pharmaceutical formulation comprising a therapeutically effective amount of SBI-553. In an aspect, a disclosed method can improve the subject's ability to move and/or level of mobility by about 10-20%, about 20-30%, about 30-40%, about 40-50%, about 50-60%, about 60-70%, about 70-80%, about 80-90%, or about 90-100% as compared to native or control levels or to the subject's ability to move and/or level of mobility prior to the administration of a disclosed composition or a disclosed pharmaceutical formulation comprising a therapeutically effective amount of SBI-553. In an aspect, a disclosed method can improve the subject's ability to move and/or level of mobility by about 0-25%, about 25-50%, about 50-75%, or about 75-100 as compared to native or control levels or to the subject's ability to move and/or level of mobility prior to the administration of a disclosed composition or a disclosed pharmaceutical formulation comprising a therapeutically effective amount of SBI-553. In an aspect, the subject's ability to move and/or level of mobility prior to the administration of a disclosed composition or a disclosed pharmaceutical formulation a therapeutically effective amount of SBI-553 can be used as a control.

In an aspect, a disclosed method can further comprise administering to the subject a therapeutically effective amount of one or more therapeutic agents. A disclosed therapeutic agent can be any disclosed agent that effects a desired clinical outcome (e.g., biologically active agents, pharmaceutically active agents, chemotherapeutic agents, anti-bacterial agents, anti-fungal agents, anti-viral agents, corticosteroids, analgesics, immunostimulants, immune-based products, or any combination thereof) and are disclosed supra.

In an aspect, the one or more therapeutic agents can be administered to the subject prior to, concurrently, or after the administration of a disclosed composition or a disclosed pharmaceutical formulation comprising a therapeutically effective amount of SBI-553.

In an aspect, a disclosed method can further comprise repeating the administering of the one or more therapeutic agents to the subject.

In an aspect, a disclosed method can further comprise monitoring the subject for adverse effects. In an aspect, in the absence of adverse effects, a disclosed method can comprise continuing to administer a disclosed composition or a disclosed pharmaceutical formulation comprising a therapeutically effective amount of SBI-553 and/or a disclosed therapeutic agent administered to the subject. In an aspect, in the presence of adverse effects, a disclosed method can comprise modifying one or more steps of a disclosed method. In an aspect, a disclosed method can further comprise modifying an aspect of the method. In an aspect, modifying an aspect of a disclosed method can comprise changing the amount of a disclosed composition or a disclosed pharmaceutical formulation comprising a therapeutically effective amount of SBI-553 and/or a disclosed therapeutic agent administered to the subject, changing the frequency of administration of a disclosed composition or a disclosed pharmaceutical formulation comprising a therapeutically effective amount of SBI-553 and/or a disclosed therapeutic agent to the subject, changing the duration of time that a disclosed composition or a disclosed pharmaceutical formulation comprising a therapeutically effective amount of SBI-553 and/or a disclosed therapeutic agent and/or a disclosed therapeutic agent is administered to a subject, or any combination thereof.

In an aspect, a disclosed method does not elicit hypothermia, hypotension, and/or motor impairment in the subject.

In an aspect, a disclosed method can further comprise administering to the subject one or more additional anti-cancer therapies. Anti-cancer therapies are known to the art. In an aspect, a disclosed anti-cancer therapy can comprise endocrine therapy, radiotherapy, hormone therapy. gene therapy, thermal therapy, ultrasound therapy, or any combination thereof. In an aspect, a disclosed anti-cancer therapy can comprise one or more chemotherapeutic agents. In an aspect, a disclosed chemotherapeutic agent can comprise an anthracycline, a vinca alkaloid, an alkylating agent, an immune cell antibody, an antimetabolite, a TNFR glucocorticoid induced TNFR related protein (GITR) agonist, a proteasome inhibitor, an immunomodulator, or any combination thereof.

In an aspect, a disclosed method comprising a therapeutically effective amount of SBI-553 can restore one or more aspects of cellular homeostasis and/or cellular functionality and/or metabolic dysregulation in a subject, such as, for example, a subject having acute and/or chronic pain. In an aspect, restoring one or more aspects of cellular homeostasis and/or cellular functionality can comprise reducing the subject's acute and/or chronic pain.

### 3. Methods Using SBI-810

Disclosed herein is a method of treating and/or preventing pain in a subject, the method comprising administering to a subject in need thereof a composition comprising a therapeutically effective amount of SBI-810, wherein pain is alleviated and/or minimized in the subject. Disclosed herein is a method of treating and/or preventing pain in a subject, the method comprising administering to a subject in need thereof a pharmaceutical formulation comprising a therapeutically effective amount of SBI-810, wherein pain is alleviated and/or minimized in the subject.

In an aspect of a disclosed method, a therapeutically effective amount of SBI-810 can comprise a dose of about 1 mg/kg/day to about 100 mg/kg/day. In an aspect of a disclosed method, a therapeutically effective amount of SBI-810 can comprise a dose of about 1 mg/kg/day to about 20 mg/kg/day, about 20 mg/kg/day to about 40 mg/kg/day, about 40 mg/kg/day to about 60 mg/kg/day, about 60 mg/kg/day to about 80 mg/kg/day, or about 80 mg/kg/day to about 100 mg/kg/day. In an aspect of a disclosed method, a therapeutically effective amount of SBI-810 can comprise a dose of about 1 mg/kg/day, about 5 mg/kg/day, about 10 mg/kg/day, about 15 mg/kg/day, about 20 mg/kg/day, about 25 mg/kg/day, about 30 mg/kg/day, about 35 mg/kg/day, about 40 mg/kg/day, about 45 mg/kg/day, about 50 mg/kg/day, about 55 mg/kg/day, about 60 mg/kg/day, about 65 mg/kg/day, about 70 mg/kg/day, about 75 mg/kg/day, about 80 mg/kg/day, about 85 mg/kg/day, about 90 mg/kg/day, about 95 mg/kg/day, or about 100 mg/kg/day. In an aspect of a disclosed method, a therapeutically effective amount of SBI-810 can comprise a dose of more than 100 mg/kg/day.

In an aspect of a disclosed method, a disclosed composition or a disclosed pharmaceutical formulation comprising a therapeutically effective amount of SBI-810 can further comprise a therapeutically effective amount of one or more therapeutic agents. A disclosed therapeutic agent can be any disclosed agent that effects a desired clinical outcome (e.g., biologically active agents, pharmaceutically active agents, chemotherapeutic agents, anti-bacterial agents, anti-fungal agents, anti-viral agents, corticosteroids, analgesics, immunostimulants, immune-based products, or any combination thereof) and are disclosed supra.

In an aspect, a disclosed method can treat and/or prevent pain in a subject. In an aspect, a disclosed method can cause the subject to experience less pain, less intense pain, a shorter duration of pain, or a combination thereof.

In an aspect, a disclosed method can improve the subject's ability to move and/or level of mobility. In an aspect, a disclosed method can improve the subject's ability to move and/or level of mobility by about 10%, about 20%, about 30%, about 40%, about 50%, about 60%, about 70%, about 80%, about 90%, or about 100% as compared to native or control levels or to the subject's level of mobility prior to the administration of a disclosed composition or a disclosed pharmaceutical formulation comprising a therapeutically effective amount of SBI-810. In an aspect, a disclosed method can improve the subject's ability to move and/or level of mobility by about 10-20%, about 20-30%, about 30-40%, about 40-50%, about 50-60%, about 60-70%, about 70-80%, about 80-90%, or about 90-100% as compared to native or control levels or to the subject's ability to move and/or level of mobility prior to the administration of a disclosed composition or a disclosed pharmaceutical formulation comprising a therapeutically effective amount of SBI-810. In an aspect, a disclosed method can improve the subject's ability to move and/or level of mobility by about 0-25%, about 25-50%, about 50-75%, or about 75-100 as compared to native or control levels or to the subject's ability to move and/or level of mobility prior to the administration of a disclosed composition or a disclosed pharmaceutical formulation comprising a therapeutically effective amount of SBI-810. In an aspect, the subject's ability to move and/or level of mobility prior to the administration of the disclosed composition or a disclosed pharmaceutical formulation comprising a therapeutically effective amount of SBI-810 can be used as a control.

In an aspect, a disclosed method does not elicit hypothermia, hypotension, and/or motor impairment in a subject.

In an aspect, a disclosed method of treating and/or preventing pain in a subject can further comprise repeating the administering of a disclosed composition or a disclosed pharmaceutical formulation comprising a therapeutically effective amount of SBI-810 to the subject.

In an aspect of a disclosed method, administering a disclosed composition or a disclosed formulation comprising a therapeutically effective amount of SBI-810 can comprise systemic or direct administration. In an aspect, administering a disclosed composition or a disclosed pharmaceutical formulation comprising a therapeutically effective amount of SBI-810 can comprise oral administration, intravenous administration, intra-articular administration, epidural administration, intrathecal administration, topical administration (e.g., skin patch), depot administration, intraperitoneal administration, or any combination thereof. In an aspect, a disclosed composition or a disclosed pharmaceutical formulation comprising a therapeutically effective amount of SBI-810 can be formulated for administration via topical administration (e.g., skin patch) or depot administration. In an aspect, administering a disclosed composition or a disclosed pharmaceutical formulation comprising a therapeutically effective amount of SBI-810 can be administered by any method of administration disclosed herein. In an aspect, a disclosed composition or a disclosed pharmaceutical formulation comprising a therapeutically effective amount of SBI-810 can be administered via multiple routes either concurrently or sequentially. For example, in an aspect, a disclosed composition or a disclosed pharmaceutical formulation comprising a therapeutically effective amount of SBI-810 can be first administered orally and then be administered intravenously. In an aspect, administering a disclosed composition or a disclosed pharmaceutical formulation comprising a therapeutically effective amount of SBI-810 can be first administered intravenously and then be administered orally. A skilled clinician can determine the best route of administration for a subject at a given time.

In an aspect of a disclosed method, administering a disclosed composition or a disclosed pharmaceutical formulation comprising a therapeutically effective amount of SBI-810 and/or a disclosed therapeutic agent can comprise continuous administration, non-continuous administration, or intermittent administration. In an aspect, continuous administration can comprise the use of an infusion pump.

In an aspect, a subject can be in the military or can be a veteran. In an aspect, a subject can have acute pain, chronic pain, or both. In an aspect, a subject's acute and/or chronic pain can comprise back pain (including upper back pain and/or lower back pain), cancer pain, arthritis pain, headaches (including but not limited to migraines, cluster headaches, and tension headaches), neuralgias, neuropathies, pain due to a CNS injury, pain due to a PNS injury, pain with no known etiological cause, or any combination thereof.

In an aspect, a subject can have one or more bone fractures. In an aspect, the one or more bone fractures can be the result of a non-trauma, a disease, or a disorder. In an aspect, a subject can be diagnosed with or can be suspected of having a bone fracture or more than one bone fractures. In an aspect the subject can have multiple fractures in the same bone, multiple fractures in different bones, or both. In an aspect of a disclosed method, a fracture can comprise an open fracture, a closed fracture, a partial fracture, a complete fracture, a stable fracture, or a displaced fracture. In an aspect of a disclosed method, a fracture comprises a transverse fracture, a spiral fracture, a greenstick fracture, a stress fracture, a compression fracture, an oblique fracture, an impacted fracture, a segmental fracture, a comminuted fracture, or an avulsion fracture. In an aspect, a subject can have one or more types of fractures. In an aspect, a disclosed method can comprise identifying the bone or bones having a fracture or fractures. In an aspect, a subject can have non-fracture pain. In an aspect, a subject can have sustained one or more injuries and/or one or more physical injuries.

In an aspect, a subject can have experienced one or more surgical procedures. In an aspect, a subject can have opioid dependence or can have recovered from an opioid dependence.

In an aspect, a subject can have been diagnosed with cancer. In an aspect, cancer can comprise ovarian cancer, ovarian adenocarcinoma, ovarian teratocarcinoma, lung cancer, small cell lung cancer (SCLC), non-small cell lung cancer (NSCLC), squamous cell lung carcinoma, adenocarcinoma, gastric cancer, breast cancer, hepatic cancer, pancreatic cancer, skin cancer, in particular basal cell carcinoma and squamous cell carcinoma, malignant melanoma, head and neck cancer, malignant pleomorphic adenoma, sarcoma, synovial sarcoma, carcinosarcoma, bile duct cancer, bladder cancer, transitional cell carcinoma, papillary carcinoma, kidney cancer, renal cell carcinoma, clear cell renal cell carcinoma, papillary renal cell carcinoma, colon cancer, small bowel cancer, small bowel adenocarcinoma, adenocarcinoma of the ileum, testicular embryonal carcinoma, placental choriocarcinoma, cervical cancer, testicular cancer, testicular seminoma, testicular teratoma, embryonic testicular cancer, uterine cancer, teratocarcinoma, embryonal carcinoma, or any combination thereof.

In an aspect, a subject can be a non-human mammal or a human. A subject can be of any age, such as, for example, a geriatric, an adult, an adolescent, a child, or a baby.

In an aspect, a disclosed method can further comprise assessing the subject's pain. The subject's pain can be assessed subjectively or objectively as known to the art.

In an aspect of a disclosed method, following the administering step, the subject can experience less pain, less intense pain, a shorter duration of pain, or a combination thereof.

In an aspect, a subject's pain can be treated and/or prevented by diminishing the intensity, the duration, the amount, or a combination thereof of a subject's pain (e.g., acute and/or chronic pain). In an aspect, a disclosed method can reduce a subject's pain by about 10%, about 20%, about 30%, about 40%, about 50%, about 60%, about 70%, about 80%, about 90%, or about 100% as compared to native or control levels or to a subject's level prior to the onset of pain (e.g., acute and/or chronic pain due to trauma, injury, cancer, disease and/or disorder, neuropathic pain, etc.). In an aspect, a disclosed method can reduce a subject's pain by about 10-20%, about 20-30%, about 30-40%, about 40-50%, about 50-60%, about 60-70%, about 70-80%, about 80-90%, or about 90-100% as compared to native or control levels or to a subject's level prior to the onset of pain (e.g., acute and/or chronic pain due to trauma, injury, cancer, disease and/or disorder, neuropathic pain, etc.). In an aspect, a disclosed method can reduce a subject's pain by about 0-25%, about 25-50%, about 50-75%, or about 75-100% as compared to native or control levels or to a subject's pain level prior to the onset of pain (e.g., acute and/or chronic pain due to trauma, injury, cancer, disease and/or disorder, neuropathic pain, etc.). In an aspect, a subject's pain level prior to the onset of pain can be used as a control.

In an aspect, a subject's acute and/or chronic pain can comprise back pain (including upper back pain and/or lower back pain), cancer pain, arthritis pain, headaches (including but not limited to migraines, cluster headaches, and tension headaches), neuralgias, neuropathies, pain due to a CNS injury, pain due to a PNS injury, pain with no known etiological cause, or any combination thereof. In an aspect, a subject's pain can be cancer pain. In an aspect, a subject's pain can be arthritis pain. In an aspect, a subject's pain can be headache pain. In an aspect, a subject's pain can be neuralgia pain. In an aspect, a subject's pain can be neuropathy pain. In an aspect, a subject's pain can be due to an injured sustained while serving in the armed forces or military.

In an aspect, a disclosed method can further comprise improving a subject's ability to move and/or level of mobility. Any improvement in a subject's ability to move and/or level of mobility can be assessed subjectively or objectively as known to the art. In an aspect, a disclosed method can improve a subject's ability to move and/or level of mobility by about 10%, about 20%, about 30%, about 40%, about 50%, about 60%, about 70%, about 80%, about 90%, or about 100% as compared to native or control levels or to the subject's ability to move and/or level of mobility prior to the administration of a disclosed composition or a disclosed pharmaceutical formulation comprising a therapeutically effective amount of SBI-810 ligand. In an aspect, a disclosed method can improve the subject's ability to move and/or level of mobility by about 10-20%, about 20-30%, about 30-40%, about 40-50%, about 50-60%, about 60-70%, about 70-80%, about 80-90%, or about 90-100% as compared to native or control levels or to the subject's ability to move and/or level of mobility prior to the administration of a disclosed composition or a disclosed pharmaceutical formulation comprising a therapeutically effective amount of SBI-810. In an aspect, a disclosed method can improve the subject's ability to move and/or level of mobility by about 0)-25%, about 25-50%, about 50-75%, or about 75-100 as compared to native or control levels or to the subject's ability to move and/or level of mobility prior to the administration of a disclosed composition or a disclosed pharmaceutical formulation comprising a therapeutically effective amount of SBI-810. In an aspect, the subject's ability to move and/or level of mobility prior to the administration of a disclosed composition or a disclosed pharmaceutical formulation can be used as a control.

In an aspect, a disclosed method can further comprise administering to the subject a therapeutically effective amount of one or more therapeutic agents. A disclosed therapeutic agent can be any disclosed agent that effects a desired clinical outcome (e.g., biologically active agents, pharmaceutically active agents, chemotherapeutic agents, anti-bacterial agents, anti-fungal agents, anti-viral agents, corticosteroids, analgesics, immunostimulants, immune-based products, or any combination thereof) and are disclosed supra.

In an aspect, one or more therapeutic agents can comprise a therapeutically effective amount of a STING agonist, a steroid, an opioid analgesic, a local anesthetic, an NSAID, a programmed cell death I ligand, an anti-cancer agent, and any combination thereof.

In an aspect, a disclosed opioid analgesic can comprise codeine, dextropropyoxyphene, diamorphine, dihydrocodeine, meptazinol, methadone, morphine, nalbuphine, pentazocine, oxycodone, and any combination thereof.

In an aspect, the one or more therapeutic agents can be administered to the subject prior to, concurrently, or after the administration of a disclosed composition or a disclosed pharmaceutical formulation comprising SBI-810.

In an aspect, a disclosed method can further comprise repeating the administering of the one or more therapeutic agents to the subject. In an aspect, a disclosed method can further comprise monitoring the subject for adverse effects. In an aspect, in the absence of adverse effects, a disclosed method can comprise continuing to administer a disclosed composition or a disclosed pharmaceutical formulation comprising a therapeutically effective amount of SBI-810 and/or a disclosed therapeutic agent. In an aspect, in the presence of adverse effects, a disclosed method can comprise modifying one or more steps of a disclosed method. In an aspect, a disclosed method can further comprise modifying an aspect of the method. In an aspect, modifying an aspect of a disclosed method can comprise changing the amount of a disclosed composition or a disclosed pharmaceutical formulation comprising SBI-810 and/or a disclosed therapeutic agent administered to the subject, changing the frequency of administration of a disclosed composition or a disclosed pharmaceutical formulation comprising SBI-810 and/or a disclosed therapeutic agent to the subject, changing the duration of time that a disclosed composition or a disclosed pharmaceutical formulation comprising SBI-810 and/or a disclosed therapeutic agent is administered to a subject, or any combination thereof.

In an aspect, a disclosed method does not elicit hypothermia, hypotension, and/or motor impairment in a subject.

In an aspect, a disclosed method can further comprise administering to the subject one or more additional anti-cancer therapies. Anti-cancer therapies are known to the art. In an aspect, a disclosed anti-cancer therapy can comprise endocrine therapy, radiotherapy, hormone therapy, gene therapy, thermal therapy, ultrasound therapy, or any combination thereof. In an aspect, a disclosed anti-cancer therapy can comprise one or more chemotherapeutic agents. In an aspect, a disclosed chemotherapeutic agent can comprise an anthracycline, a vinca alkaloid, an alkylating agent, an immune cell antibody, an antimetabolite, a TNFR glucocorticoid induced TNFR related protein (GITR) agonist, a proteasome inhibitor, an immunomodulator, or any combination thereof.

In an aspect, a disclosed method comprising a therapeutically effective amount of SBI-810 can restore one or more aspects of cellular homeostasis and/or cellular functionality and/or metabolic dysregulation in a subject, such as, for example, a subject having acute and/or chronic pain. In an aspect, restoring one or more aspects of cellular homeostasis and/or cellular functionality can comprise reducing the subject's acute and/or chronic pain.

G. Miscellaneous

Disclosed herein is a non-opioid analgesic for the treatment of multiple forms of acute and chronic pain, the non-opioid analgesic compound comprising a β-arresting-biased allosteric modulator of NTSR1 compound or a pharmaceutically acceptable salt, solvate, hydrate, prodrug, ester, or derivative thereof. In an aspect, a disclosed modulator can be selected from the group consisting of SBI-555, SBI-810, and combinations thereof.

Disclosed herein is a non-opioid analgesic for the treatment of multiple forms of acute and chronic pain, the non-opioid analgesic comprising a β-arresting-biased allosteric modulator of NTSR1 modulator compound having the general formula (I) (termed SBI-553):

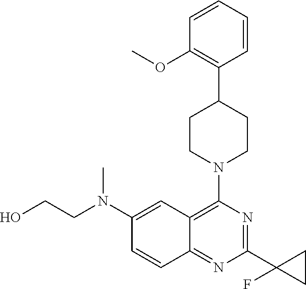

or a pharmaceutically acceptable salt, solvate, hydrate, prodrug, ester, or derivative thereof.

Disclosed herein is non-opioid analgesic for the treatment of multiple forms of acute and chronic pain, the non-opioid analgesic comprising a β-arresting-biased allosteric modulator of NTSR1 modulator compound having the general formula (II) (termed SBI-810 or SBI-0653810):

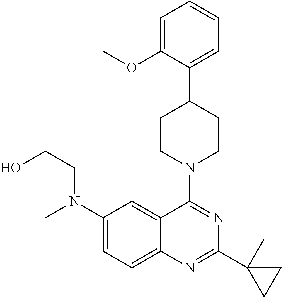

or a pharmaceutically acceptable salt, solvate, hydrate, prodrug, ester, or derivative thereof.

Disclosed herein is a pharmaceutical composition comprising a compound as in any of the preceding claims and a pharmaceutically acceptable carrier and/or excipient.

Disclosed herein is a method of treating acute and/or chronic pain in a subject, the method comprising administering to the subject a therapeutically effective amount of a compound as in any of the preceding claims such that the acute and/or chronic pain is treated in the subject.

Disclosed herein is a method of preventing acute and/or chronic pain in a subject, the method comprising administering to the subject a therapeutically effective amount of a disclosed compound in which the acute and/or chronic pain is prevented in the subject.

In an aspect, a disclosed method can further comprise administering to the subject at least one additional therapeutic agent. In an aspect, a disclosed additional therapeutic agent can be selected from the group consisting of a STING agonist, a steroid, an opioid analgesic, a local anesthetic, an NSAID, a programmed cell death 1 ligand, an anti-cancer agent, and combinations thereof. In an aspect, a disclosed compound can be administered prior to the at least one additional therapeutic agent. In an aspect, a disclosed compound can be administered concurrently with the at least one additional therapeutic agent. In an aspect, a disclosed compound can be administered after the at least one additional therapeutic agent.

Disclosed herein is a method of preventing and/or alleviating opioid addiction in a subject. the method comprising administering to the subject a therapeutically effective amount of a disclosed compound and an opioid analgesic. In an aspect, a disclosed opioid analgesic can be selected from the group consisting of codeine, dextropropyoxyphene, diamorphine, dihydrocodeine, meptazinol, methadone, morphine, nalbuphine, pentazocine, oxycodone, and combinations thereof.

### EXAMPLES

Neurotensin is a natural peptide system that regulates a myriad of biological functions in humans and animals. While neurotensin was discovered in the 1970, attempts to develop small molecule ligands by the pharmaceutical industry have all failed, as conventional activation of NTSR1 is plagued with untoward side effects associated with NTSR1 agonism, such as hypotension, hypothermia and motor coordination impairments. However, research has shown that like all other GPCRs, NTSR1 signals not only through heterotrimeric G proteins but also through of the β-arrestins which were previously thought to simply terminate G protein signaling. But, as shown in FIG. 2, chemically synthesized small molecules can selectively engage either of the signaling pathways to mediate distinct cellular functions of NTSR1 (Slosky L M, et al. (2020) Cell. 181(6) 1364-1379e14). Interestingly, a functionally selective β-arrestin biased ligand (SBI-553) for the NTSR1 can markedly inhibits not only the motor enhancing effects of psychostimulants like cocaine and amphetamine but also the propensity to self-administer. Most significantly, however, SBI-553 also totally eliminated the side effects normally associated with G protein activation of NTSR1. Notably, β-arrestin-2 in the spinal cord contributes to the resolution of chronic pain, in part through modulation of NMDA receptor (NMDAR) function in spinal cord dorsal horn (SCDH) neurons (Chen G, et al. (2016) Nat Commun. 7:12531). The recent development of functionally selective/biased ligands for GPCRs represents an unprecedented opportunity to develop safer, more effective and selective modulation of biological functions such as pain management.

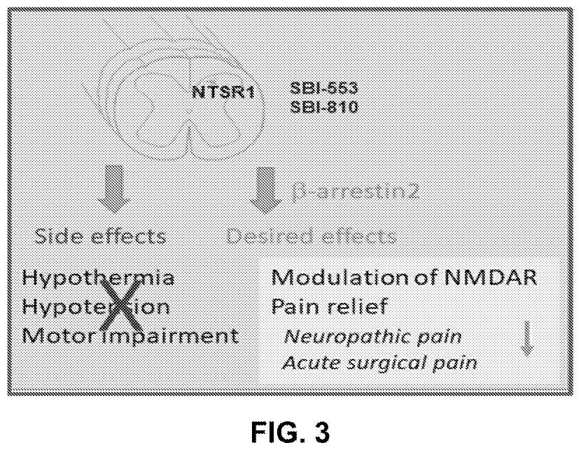

As multifunctional scaffold proteins, β-arrestins (Arrb1 and Arrb2) regulate the endocytosis, signaling, trafficking, and ubiquitination of GPCRs (Reiter E, et al. (2012) Annu. Rev. Pharmacol. Toxicol. 52:179-197). Arrb2 was shown to modulate m-opioid receptor (MOR) desensitization, and mice lacking Arrb2 exhibited enhanced and prolonged morphine analgesia (Chen G, et al. (2016) Nat Commun. 7:12531: Bohn L M, et al. (1999) Science. 286(5449):2495-2498). Arrb2 also causes desensitization of TRPV1, a key player in inflammatory pain (Por ED, et al. (2012) J Biol Chem. 287(44):37552-37563). Activation of NMDAR in SCDH nociceptive neurons plays an essential role in driving pain hypersensitivity after painful insults (Woolf C J, et al. (2000) Science. 288(5472):1765-1769). Notably, Arrb2 deficiency leads to increased surface expression and hyperactivity of the NMDAR GluN2B subunit in spinal lamina IIo neurons in association with chronic pain states. Furthermore, acute pain and chronic pain can be arrested by Arrb2, as acute inflammatory pain and chronic neuropathic pain are prolonged in mice lacking Arrb2 (Chen G, et al. (2016) Nat Commun. 7:12531). Increasing Arrb2 expression in the spinal cord is sufficient to revolve chronic neuropathic pain (Chen G, et al. (2016) Nat Commun. 7:12531). Whether selective activation of β-arrestin-2 by the biased NTSR1 agonists can alleviate neuropathic pain and acute surgical pain via NTSR1 and modulation of NMDAR was investigated. Preliminary data showing significant analgesic actions of SBI-553 in mouse models of neuropathic pain and surgical pain encouraged this investigation (FIG. 3).

In addition to the central modulation in the spinal cord/brain, NT receptors were also expressed in primary sensory neurons of dorsal root ganglion (DRG) of rats, and application of NT to dissociated nociceptor DRG neurons evoked outward currents, suggesting an inhibition on nociceptor excitability (Zhang X, et al. (1995) J Neurosci. 15(4):2733-2747). Notably, β-arrestin-2 is present in DRG neurons and inhibits pre-synaptic NMDAR activity in spinal axonal terminals derived from DRG neurons (Chen G, et al. (2016) Nat Commun. 7:12531). Whether biased agonists produce pain relief via a peripheral mechanism in DRG nociceptor neurons was also investigated. Human DRG preparation will also enhance the translational potential of this project.

The Examples that follow are illustrative of specific aspects of the invention, and various uses thereof. They set forth for explanatory purposes only and are not to be taken as limiting the invention.

### A. Materials and Methods Employed in Specific Examples

### 1. Animals

C57BL/6 mice and SD rats (7-26 weeks old) are used for behavioral, biochemical, and electrophysiological experiments. All the experiments are performed in both males and females to compare sex-dependent effects. In all studies, littermates of the same sex and genotype were randomly assigned to experimental groups. Knockout mouse lines for Arrb1, Arrb2, NTSR1, and NTSR2 on a C57BL/6 background are used. Mice were maintained on a 12:12 hour light: dark cycle.

### 2. Models of Peripheral Neuropathy, Nerve Trauma, and Incision

A mouse CIPN model is generated with 4 injections of paclitaxel (4×PTX, 2 mg/kg, IP, on days 0, 2, 4, and 6). The SNI model is as previously described (Chen G, et al. (2015) J Clin Invest. 125(8):3226-3240). Mice or rats are anesthetized with isoflurane, and a 5.0 silk tight ligation of the tibial and common peroneal nerves is performed, followed by transection and removal of a 3-to 5-mm portion of the nerve, leaving the third peripheral branch of the sciatic nerve (the sural nerve) intact (Tao X, et al. (2021) Front Physiol. 12:687046).

For mouse plantar incision, a 0.5-cm longitudinal incision of the plantar aspect of the hind paw; beginning 0.5 cm from the end of the heel, is made with a surgical blade through the skin, fascia, and plantaris muscle (Brennan T J, et al. (1996) Pain. 64(3):493-501: Yamakita S, et al. (2017) Neuroscience. 361:58-68). For intrathecal injection, spinal cord puncture is made with a 30G needle between L5/L6 level to deliver reagents (10 μL) to the cerebral spinal fluid (Hylden J L, et al. (1980) Eur. J. Pharmacol. 67(2-3):313-316). Whole blood is collected from the submandibular branch of the jugular vein.

### 3. Behavioral Tests

All behavioral tests are performed in a blinded manner. When possible, behavioral tests are conducted in the morning (8 AM-11:30 AM). Mechanical sensitivity tests are performed in boxes on an elevated metal mesh floor under stable room temperature and humidity. Mice are habituated to the environment for 2 days. The plantar surface of the left hind-paw is stimulated using a series of von Frey fibers with logarithmically increasing stiffness (0.02 gram-2.56 gram, Stoelting), and 50% paw withdrawal threshold (PWT) is determined following Dixon's up-down method (Dixon W J (1980) Annu. Rev. Pharmacol. Toxicol. 20:441-462: Chaplan S R, et al. (1994) J. Neurosci Methods. 53(1):55-63). The frequency response is measured by stimulating the hind-paw with a 0.16 or 0.4 gram von Frey hair for ten times and the percentage withdrawal response are calculated as frequency.

An acetone test for cold allodynia is performed. Through the mesh floor two acetone applications (20 μL/application) are gently applied to the bottom of a hindpaw using a pipette. Responses to acetone are graded to the following 4-point scale: 0 (no response), 1 (quick withdrawal, flick or stamp of the paw), 2 (prolonged withdrawal or repeated flicking of the paw), and 3 (repeated flicking of the paw with licking directed at the ventral side of the paw) (Luo X, et al. (2019) J Neurosci. 39(35):6848-6864).

Rota-rod test (IITC Life Science Inc.) is used to assess motor function. Mice are tested for three trials separated by 10 min intervals. During the tests, the speed of rotation is accelerated from 4 rpm to 40 rpm in 5 min. The falling latency is recorded.

### 4. Operant Behavioral Testing of Non-Reflexed Ongoing Pain

Conditioned place preference (CPP) test is as previously described (Xu Z Z, et al. (2015) Nature medicine. 21(11):1326-31: (King T, et al. (2009) Nat. Neurosci. 12(11):1364-1366). All mice are subjected to a 3-day pre-conditioning habituation period and animal behavior is video-recorded on day 3. On the conditioning day, mice receive the vehicle control paired with a randomly chosen chamber in the morning, and then the appropriate drug (e.g., SBI compound) treatment paired with the other chamber. On the test day, mice are placed in the CPP box with access to both chambers. Animal behavior is video-recorded for 15 minutes and analyzed by ANY-maze software for chamber preference (Xu Z Z, et al. (2015) Nature medicine. 21(11):1326-31).

Conditioned place aversion (CPA) is used. Negative valence associated with painful stimuli (e.g., von Frey hair, acetone) is assessed using CPA. A biased compartment assignment paradigm (two-chamber, dark and bright) is used. The effect of alternative treatment on the time of mice spending in the dark compartment receiving conditional stimuli is measured (Donnelly C R, et al. (2021) Nature. 591(7849):275-280).

Guarding behavior (spontaneous pain) after plantar incision is assessed by the guarding pain scale (score of 0-2) every 5 min for 30 min (Yamakita S, et al. (2017) Neuroscience. 361:58-68).

Open-field testing is used to analyze animal movement. The Accuscan open field (Omnitech Electronics, Inc.) was used to assess locomotor activity as previously described (Toth et al., 2018: Slosky L M, et al. (2020)). Here, animal movement is determined by the breaking of gridded photocell beams.

Anxiety-like behavior is measured using the elevated plus maze (EPM). The EPM has 2open and 2 closed arms elevated 50 cm above the ground. Anxiety behavior is assessed by time and distance traveled within the open arms using ANY-maze.

### 5. Confocal β-Arrestin 2 Translocation Assay

The translocation of β-arrestin2 to the membrane by NTSR1 was assessed using a U2OS cell line permanently expressing the NTSR1 and green fluorescent protein (GFP) tagged-β-arrestin2, as described (Barak et al., 2016). Briefly, on day 1, stable cells were split into MGB101-1-2-LG glass-bottom 384-well plates (MatriCal, Spokane, WA, USA). Each well contained 30) mL aliquots of 8,000 cells in Minimum Eagle's medium (MEM) containing 10% fetal bovine serum (FBS) and 100 U/mL penicillin/streptomycin (Life Technologies, Grand Island, NY, USA). The plates were incubated overnight at 37°° C, in 5% CO₂, and on the following day the media was changed to 30 mL serum-and phenol red-free MEM. Increasing concentrations of 10×NTS, SBI-553 and SR142948A stocks were added to the wells and diluted 10-fold to reach final concentrations. To test the additive/antagonist property of NTSR1 ligands, cells were pretreated with SBI-553 (2 or 4 mM) or SR142948A (100 pM-10 mM) 10 min prior to treatment with NTS (100 pM-100 mM) or SBI-553 (10 mM). After treatment, plates were returned to the incubator for 40 min. The cells were then fixed by adding 30 mL of 2% paraformaldehyde-phosphate buffered saline (PBS) to each well. Plates were stored at 4° C, until analysis at 488 nm on a robotic imager. Images were analyzed using a wavelet algorithm to measure formation of fluorescence aggregates (Evron et al., 2014). Data are represented as mean aggregates per unit cell area±SEM. Analyzed image results were visually confirmed. In sample images, brightness and contrast were increased uniformly across all images and all conditions to optimize visualization of GFP signal. To determine whether observed β-arrestin2 translocation was NTSR1-dependent, representative images were captured from U2OS cells transiently expressing GFP tagged β-arrestin2 and human NTSR1 or empty pcDNA vector. For these experiments, cells were plated at a density of (4-8)×10+/well in 35 mm MatTek (Ashland, MA, USA) glass coverslip dishes. Cells were left unstimulated or treated with 200 nM NTS or 7.5 mM of SBI-553 for 40 min, fixed in 2% paraformaldehyde, and examined on a Zeiss Axiovert 200 fluorescence microscope using a plan-apochromat 40×/0.95 N.A, air objective.

### 6. Core Body Temperature Measurement

Core body temperatures were recorded from age-matched male and female C57BL/6J mice prior to treatment (Time 0) and 30 min, 60 min, 120 min, and 300 min post-treatment using a rectal probe thermometer for mice (Thermalert Model TH-8, Physitemp Instruments, Inc., Clifton, NJ). On three days of the week leading up to the study, animals were handled and temperatures were taken to acclimate the animals to the procedure.

### 7. Biochemical and Histochemical Studies

After terminal anesthesia, animals are transcardially perfused with PBS (biochemistry assay's) or with PBS followed by 4% paraformaldehyde (histochemical assays).

### 8. Discomfort, Distress, and Injury

Previous experience with these protocols and processes demonstrates that the overall health status of the animals during the survival period is excellent. If there is an indication that the severity of the procedure is disturbing the animal such as motor impairment, dramatic weight loss, dramatic decrease in grooming, and aggressive behaviors, then the experiment is terminated and the animal is euthanized. Veterinary care is provided as needed.

### 9. Euthanasia

Euthanasia is performed by methods specified and approved by IACUC of Duke University Medical Center and in accordance with recommendations by American Veterinary Medical Association. For most behavioral studies, mice are euthanized in a sealed CO₂ chamber at the Animal Facility. Euthanasia is conducted by well trained technicians hired by the animal facility. For tissue harvest, mice are terminally anesthetized with isoflurane (≈10%) in a sealed chamber, followed by a quick decapitation with sharp scissors. For biochemical and histochemical studies, mice are terminally anesthetized with isoflurane (≈10%) in a sealed chamber, followed by transcardial perfusion with PBS or 4% paraformaldehyde.

### B. Specific Examples

### Preliminary Studies

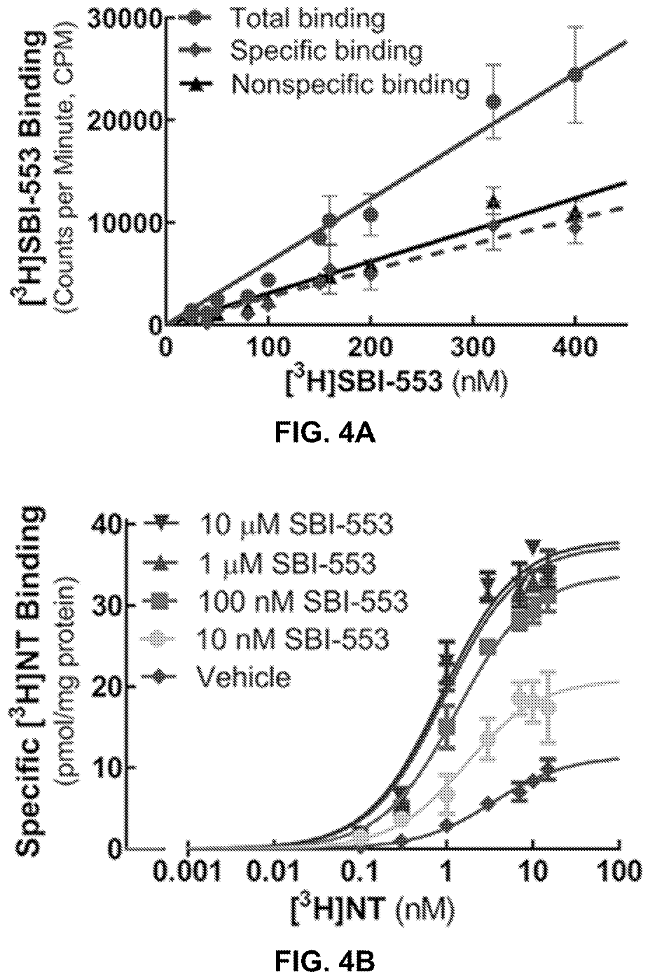

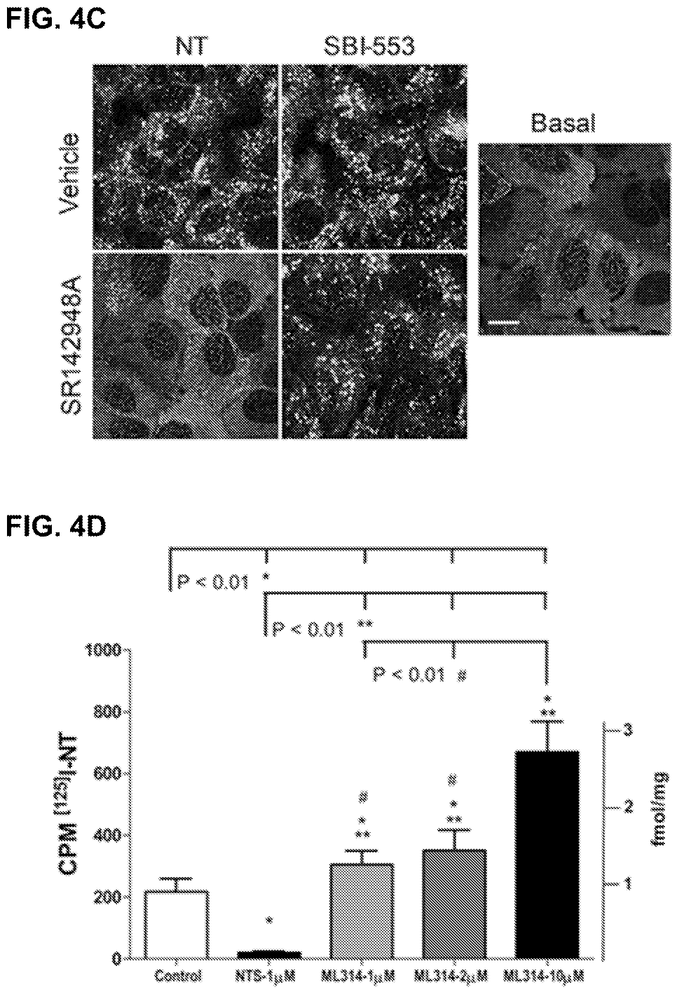

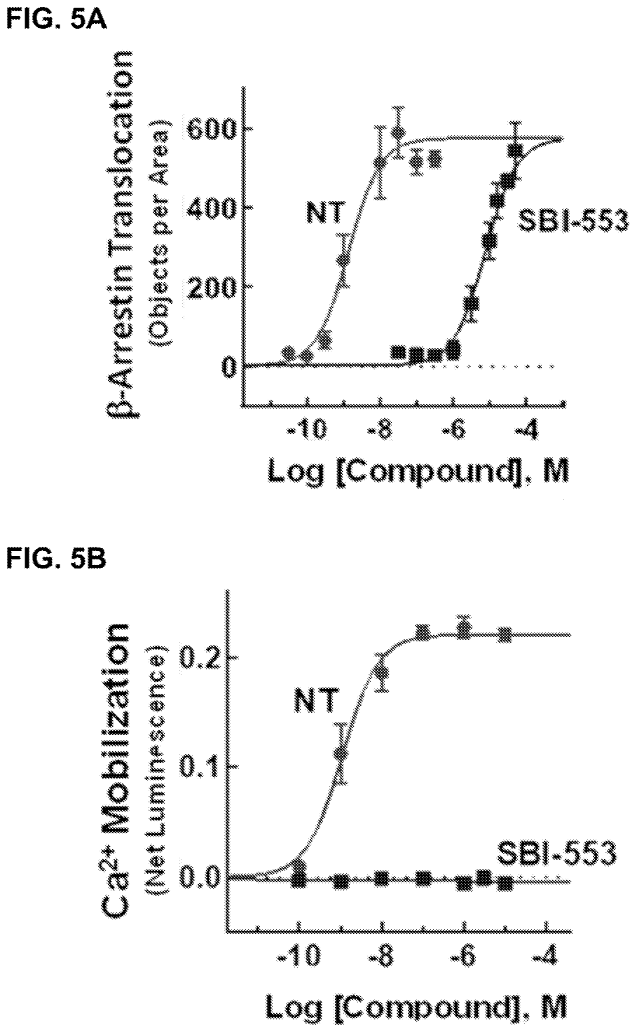

Preliminary studies provided significant data for this example. SBI-553 was derived from a medicinal chemistry campaign to improve the ML314 probe series and was selected for potency, pharmacokinetic profile, and selectivity for NTSR1 over the NTSR2 (Pinkerton A B, et al. (2019) J Med Chem. 62(17):8357-8363, which is incorporated by reference for its teachings of the making of SBI-553). SBI-553 binds NTSR1 at an allosteric site and exhibits the binding characteristics of a positive allosteric modulator (PAM). Specific [³H]SBI-553 binding comprised ˜50% of total binding below 400 nM, but nonspecific binding at higher concentrations precluded an affinity (K_{d}) determination (FIG. 4A). Binding specificity to NTSR1 was further confirmed by comparing membranes containing an off-target receptor (i.e., b2-adrenergic receptor) (Slosky L M, et al. (2020) Cell. 181(6):1364-1379e14). Notably, [³H]SBI-553 was not displaced from NTSR1 by the competitive, orthosteric NTSR1/2 antagonist SR142948A or unlabeled NTS. Rather, NTS increased total [³H]SBI-553 binding, and, conversely, SBI-553 increased total [³H]NTS binding (Slosky L M, et al. (2020) Cell. 181(6):1364-1379e14) (FIG. 4A-FIG. 4B). These behaviors are explained by the simultaneous and co-operative binding of NTS to the NTSR1 orthosteric site and SBI-553 to a distinct allosteric location (Canals M, et al. (2012) J Biol Chem. 287(1):650-659). SBI-553 behaved as a NTSR1 positive allosteric modifier (PAM), dose-dependently increasing both NTS affinity for NTSR1 (K_{d}, up to 3.9-fold) and the number of available [³H]NTS binding sites (Bₘₐₓ, up to 3.3-fold) (FIG. 4B-FIG. 4D). These findings indicate that SBI-553 is a β-arrestin-biased ligand that stimulates β-arrestin-mediated processes at NTSR1 797516251 without activating Gq protein signaling (FIG. 5). Critically, SBI-553 extended complete β-arrestin functional selectivity to NTS, an otherwise balanced, endogenous ligand.

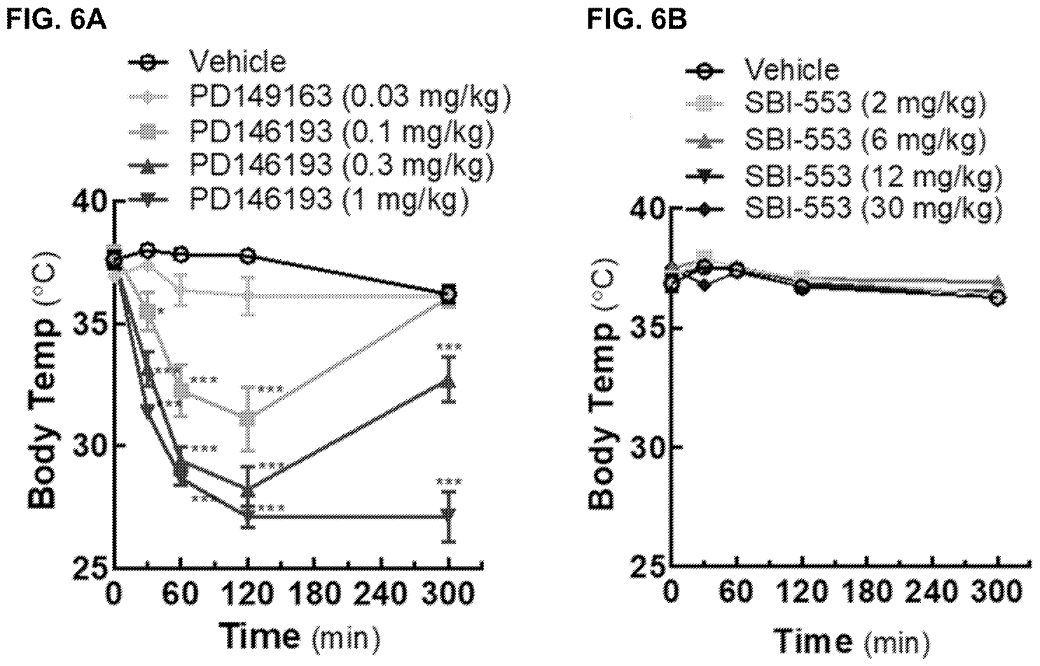

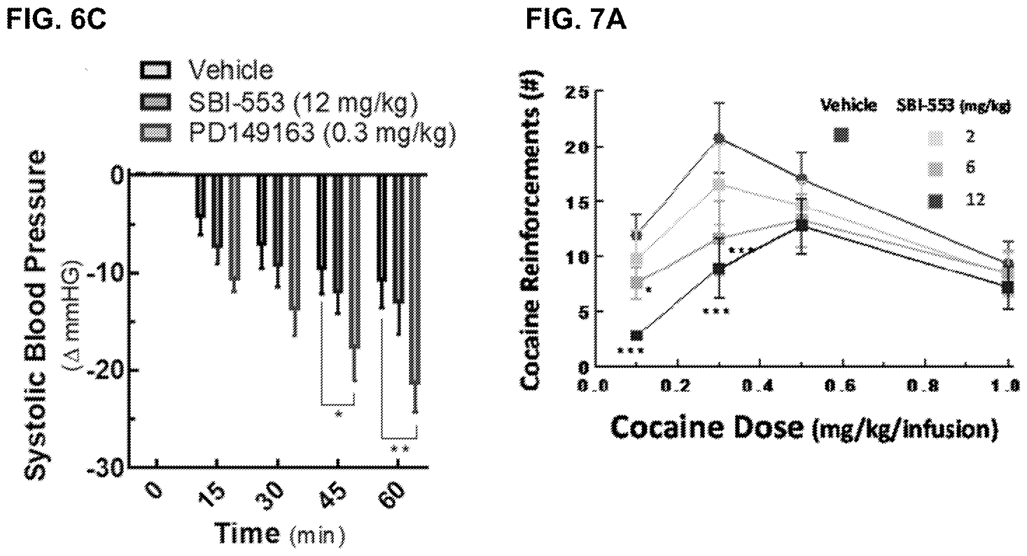

SBI-553 treatment was not associated with hypothermia or hypotension. In addition to modulating motivated behavior. NTS peptide analogs act centrally and peripherally to produce hypotension (Carraway R, et al. (1973) J Biol Chem. 248(19):6854-6861), hypothermia (Bissette G, et al. (1976) Nature. 262(5569):607-609; Kitabgi P, et al. (1992) Neuropharmacol. 15 Suppl 1 Pt A. 313A-314A), muscle relaxation, impaired motor coordination (Osbahr A J 3rd, et al. (1979) Eur J Pharmacol. 54(3):299-302: Pettibone D J, et al. (2002) J Pharmacol Exp Ther. 300(1):305-313), and sedation (Nemeroff C B (1980) Neurotensin: perchance an endogenous neuroleptic? Biol Psychiatry 15(2):283-302). To determine whether SBI-553 shares this side effect profile, the core body temperature and central blood pressure was measured in C57BL/6J mice treated with SBI-553 or PD149163. and unbiased, brain penetrant NTS 8-13 analog (Petrie K A, et al. (2004) Neuropsychopharmacology. 29(10):1878-1888: Wustrow D J, et al. (1995) Bioorg Med Chem Lett. 5(9):997-1002). PD149163 (at 0.1-1 mg/kg, i.p.) induced a rapid and long-lasting hypothermia (FIG. 6A). SBI-553 (at 2-30 mg/kg, i.p.) had no effect on core body temperature (FIG. 6B). Hemodynamic parameters showed no differences between SBI-553 (at 12 mg/kg, i.p.) and vehicle treatments whereas PD149163 (at 0.3 mg/kg, i.p.) significantly reduced peak systolic pressure at the 60 min time point (FIG. 6C). This was an effect not secondary to hypothermia as body temperature was maintained by thermal support. These findings indicate that a Gq protein-dependent mechanism mediates NTSR1-associated side effects and is not engaged by SBI-553. Consistent with an antagonist like action at NTSR1. SBI-553 pretreatment (p.o.) 30 min prior to PD149163 administration (i.p.) dose-dependently reversed the decreased in body temperature associated with PD149163.

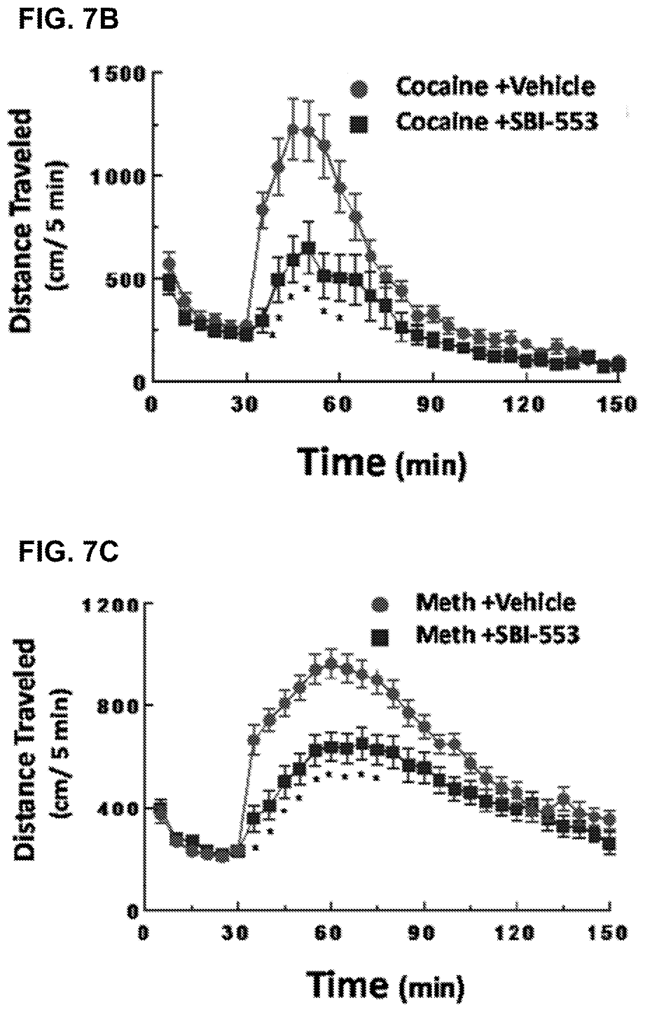

The effect of SBI-553 on cocaine self-administration was both cocaine-dose dependent and SBI-553-dose dependent. SBI-553 exhibited a typical pattern of maintenance intake reduction (Mantsch J R, et al. (2007) Psychopharmacology. 192(4):581-591), with higher SBI-553 doses showing greater efficacy at lower cocaine infusion doses (FIG. 7A). In the presence of β-arrestin2, SBI-553 reduced activity in mice. A 12 mg/kg (i.p.) dose of SBI-553 decreased both cocaine-and methamphetamine-induced hyperlocomotion in wild-type (WT) mice on a C57BL/6J background (FIG. 7B-FIG. 7C). SBI-553 also reduced methamphetamine conditioned place preference (Slosky LM, et al. (2020) Cell. 181(6):1364-1379e14). Conversely, a significant reduction in locomotion was absent in global β-arrestin2 KO mice (Slosky L M, et al. (2020) Cell. 181(6):1364-1379e14).

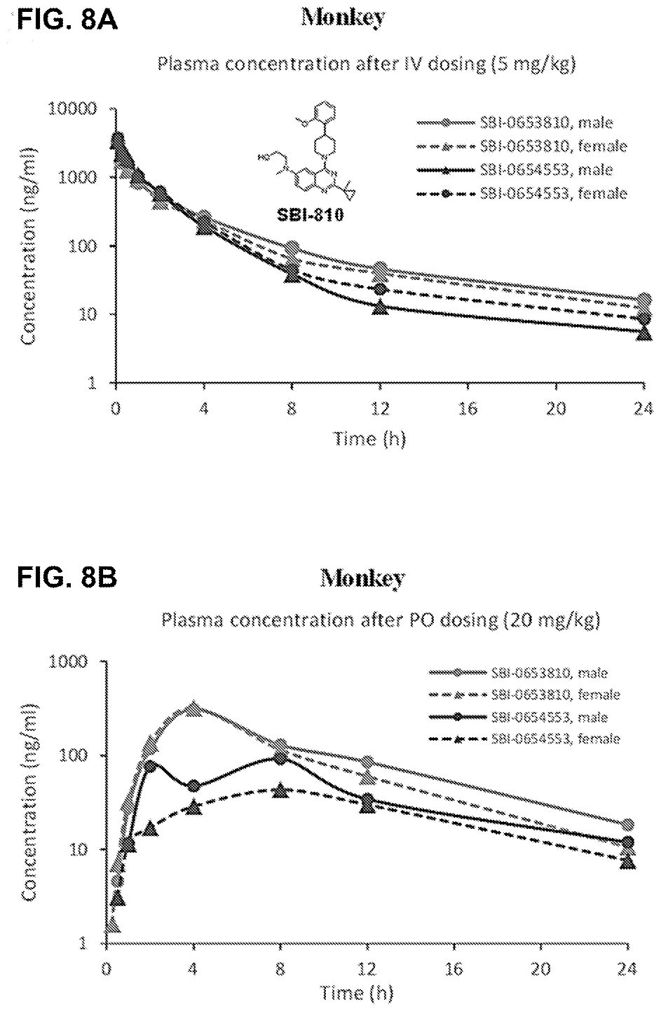

SBI-553 pharmacokinetics (PK) and pharmacodynamics (PD) were characterized in 3 species (FIG. 8A through the UG3/UH3 program. Both SBI-553 and SBI-810 shows good levels in plasma following i.v, and p.o, dosing with sustained levels being maintained over 4 hours to 8 hours (FIG. 8B).

Parameters

| | Mouse | Rat |
|---|---|---|
| compound* | SBI-553 | SBI-553 |
| CIₚ^{b} (mL/min/kg) | 44.8 | 81 |
| Vd^{b} (L/kg) | 6.16 | 7.02 |
| Cₘₐₓ^{c} (ng/mL) | 1460 | 3482 |
| AUC (ng h/ml | 4824 | 2693 |
| T_{2/2}^{c}(h) | 5.28 | 2.23 |
| % F | 45 | 48 |
| brain:plasma @ 1 h^{c} | 054 | 0.98 |

*Compounds dosed 5 mpk tv and 30 mpk po.
^{b}tv.
^{c}pa

Parameters
Monkey - IV (5 mg/kg, n = 2)

| compound | SBI-810* | SBI-553* |
|---|---|---|
| Co (ng/ml) | 4702 | 4572 |
| T_{2/2} (h) | 6.54 | 4.78 |
| Cl (mL/min/kg) | 19.2 | 19.2 |
| AUCₒ₋ₗₐₛₜ (ng · h/mL) | 4209 | 4281 |
| AUCₒ₋inf (ng · h/mL) | 4344 | 4335 |
| MRT₀₋ₗₐₛₜ (h) | 3.4 | 2.3 |
| V_{dss} (L/kg) | 5 | 3.1 |

*IV vehicle 5% DMSO 95% (10% HP-6-CD tn 10 mM citrate buffer, pH 3.5) clear solution

Parameters
Monkey - PO (20 mg/kg, n = 2)

| compound | SBI-810** | SBI-553** |
|---|---|---|
| Cₒ(ng/ml) | 314 | 67.6 |
| T_{1/2}(h) | 4 | 8 |
| CI(mL/min/kg) | 5.2 | 8 |
| AUCₒ₋ₗₐₛₜ(ng · h/mL) | 2120 | 797 |
| AUCₒ₋inf(ng · h/mL) | 2229 | 864 |
| MRT₀₋ₗₐₛₜ (h) | 8 | 11.9 |
| V_{dss}(L/kg) | 12.8 | 5 |

**PO vehicle 5% DMSO 20% Solutol 75%, 0.1 NHCl final pH 1.5 homogeneous opaque suspension

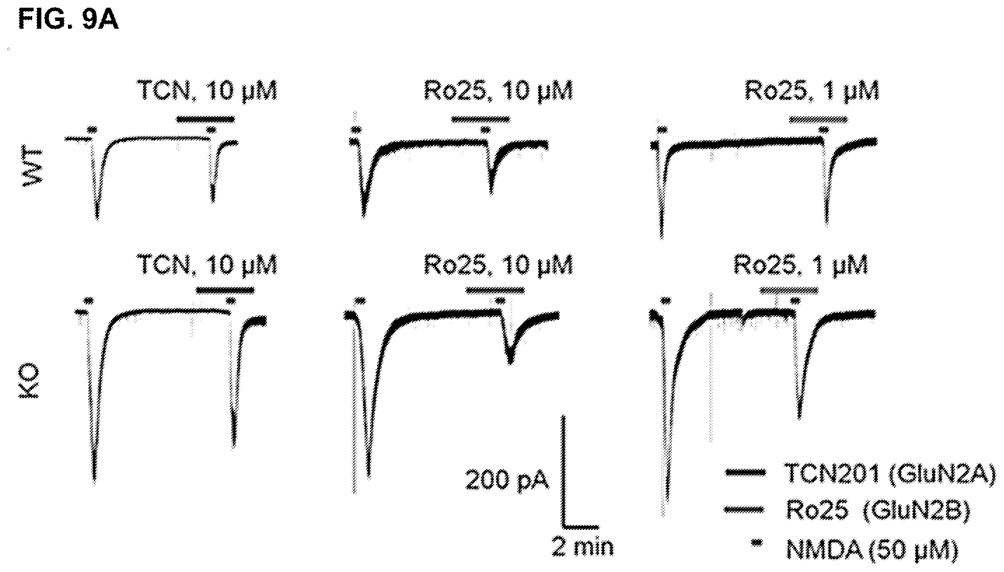

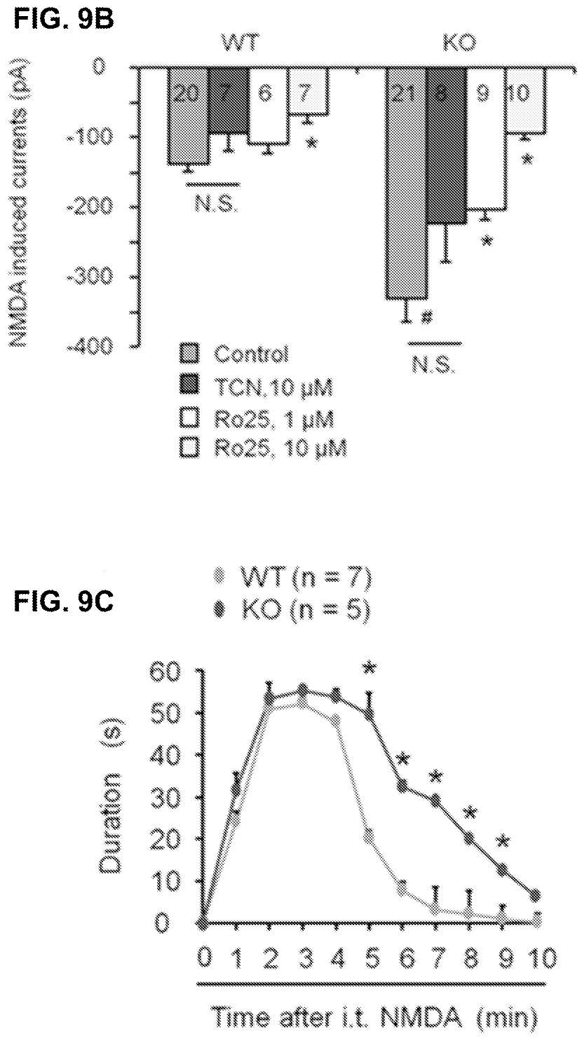

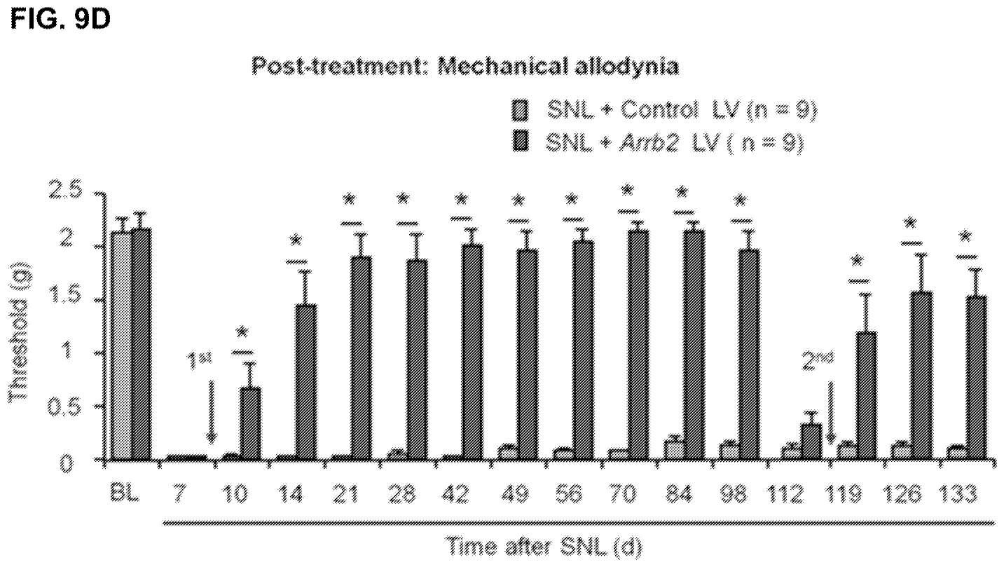

Spinal cord NMDAR plays a critical role in driving inflammatory and neuropathic pain (Woolf C J, et al. (2000) Science. 288(5472):1765-1769). Using spinal cord slice preparations, NMDA (50 mM) induced inward currents in spinal cord out lamina II (IIo) neurons were recorded in WT and Arrb2-KO mice. Arrb2 deficiency led to a 3-fold increase in NMDA current amplitude, and this change was dose-dependently blocked by NR2B antagonist Ro25 but not by NR2A antagonist TCN (FIG. 9A and FIG. 9B). Similarly, duration of intrathecal (i.t.) NMDA-induced spontaneous pain was also potentiated in the KO mice (FIG. 9C). The duration of inflammatory pain and neuropathic pain was also prolonged in KO mice (Chen G, et al. (2016) Nat Commun. 7:12531). Von Frey hair testing showed sustained mechanical allodynia (a cardinal feature of neuropathic pain) for several months after spinal nerve ligation (SNL, FIG. 9D). Strikingly, microinjection of Arrb2-lentivirus (LV) in SCDH, given in either early-phase (7 d) or late-phase (112 d) was sufficient to reverse mechanical allodynia (FIG. 9D). Collectively, these data indicated that β-arrestin-2 played an active role in the resolution process of pain by arresting NMDAR function.

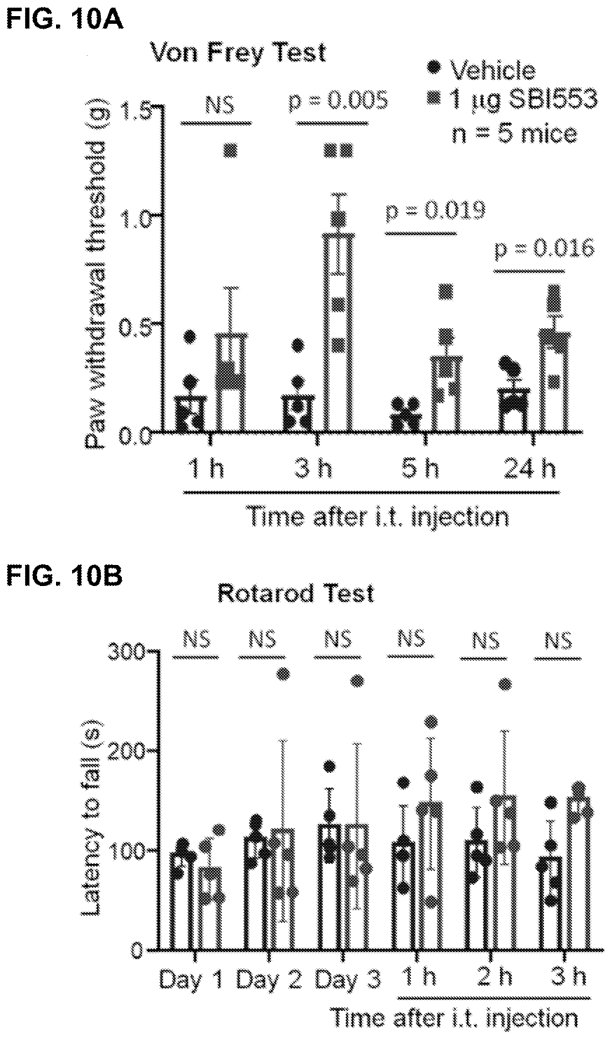

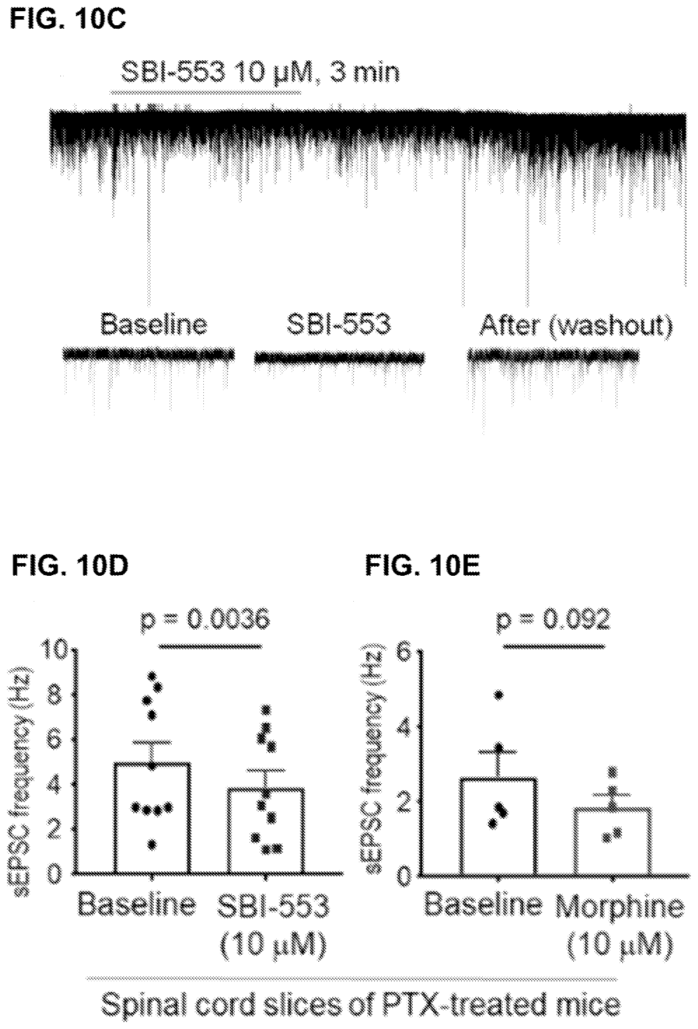

Spinal (intrathecal) administration of the SBI-553 reduces neuropathic pain and spinal cord synaptic transmission. In a pilot study, the analgesic effects of SBI-553 on neuropathic pain induced by the chemotherapy agent paclitaxel (PTX) in male mice. Mechanical allodynia using von Frey testing based on paw withdrawal threshold (PWT) was measured. Four weeks after the PTX treatment, SBI-553 was administered via intrathecal injection (i.t., 1 mg≈2 nmol), as intrathecal route can target spinal cord pain pathway. Strikingly, i.t, injection of SBI-553 produced significant reduction of mechanical allodynia for >24 hr, as shown by increased PWT, compared to vehicle (FIG. 10A). Rotarod testing showed no significant difference between SBI-553 and vehicle groups (FIG. 10B). Interestingly, SBI-553-treated mice showed a tendency to spend more time on the treadmill, due to reduced pain level (FIG. 10B). Together, these data indicated that SBI-553 produced potent and sustained pain relief without causing motor impairment.

The work using isolated spinal cord slices demonstrated that SBI-553 perfusion (10 mM, 3 min) significantly suppressed excitatory synaptic transmission, as measured by spontaneous excitatory postsynaptic currents (sEPSCs) in lamina IIo neurons (FIG. 10C-FIG. 10E). This result indicated that SBI-553 produced analgesia in the spinal cord pain circuit. Morphine suppresses pain by both pre-synaptic and post-synaptic modulation in the spinal cord pain circuit (Wang Z, et al. (2020) Sci Transl Med. 12 (531)), and its analgesic efficacy is reduced in neuropathic pain conditions (Kohno T, et al. (2005) Pain. 117(1-2):77-87). Notably, at the concentration of 10 mM, morphine did not produce significant inhibition (p=0.092) of sEPSC in spinal neurons of chemotherapy mice. These data indicated that the biased agonist SBI-553 can alleviate pain by spinal cord mechanism.

### Example 1

### Determination of Selectivity and Efficacy of SBI-553 and IND Lead Compounds

As discussed below, several tasks are performed in this determination.

First, using cellular assays, whether IND candidates such as SBI-810 are similar or better than SBI-553 as β-arrestin biased agonist at NTSR1. is assessed. The chemical entities used in this work were synthesized and validated. However, routine validation of the profile of these compounds is performed routinely using both cellular and in vivo assays. For example, HEK293 cells that express either the NTSR1 or NTSR2 are used to assess the functional profile of the various chemical probes. In addition, a series of genetically engineered mouse lines are used to provide an in vivo validation of novel chemical entities for their abilities to module behaviors in these animals. In HEK293 cells permanently expressing the NTSR1. assays comparing the characterized β-arrestin biased NTSR1 ligand SBI-553 are performed and compared with SBI-810, which was formulated to show good levels in plasma following i.v, and p.o, dosing with sustained levels being maintained over 4 hours-8 hours.

Second, whether IND candidates provide the same or better efficacy at inhibiting the addictive properties of psychostimulants and opioids in vivo is examined. The most appropriate way to determine whether IND candidates show efficacy at inhibiting the addictive properties psychostimulants or opioids is to test whether such compounds can reduce the self-administration of drugs in a drug self-administration paradigm. The properties of SBI-553 for its abilities to profoundly reduce the self-administration of both psychostimulants and opioids was characterized (Slosky L M, et al. (2020) Cell. 181(6):1364-1379e14). However, at least for psychostimulants and to some extent opioids, there are other less complex paradigms to assess the ability of a compound like SBI-553 to monitor its inhibitory effects by monitoring the ambulatory effects of psychostimulants in mice or rats. These other paradigms could be used initially if drug self-administration is not readily available. Dopamine mediated reward is commonly assessed using mouse behavioral assays like locomotion and conditioned place preference. A consistent framework within which to enroll the animal, test relevant hypotheses, employ univariate or multivariate analysis to uncover trends and identify new hypotheses, and identify biological mechanisms that can be extrapolate to humans are developed. Open field activity in the presence of amphetamine and cocaine are carried out using C57BL/6J mice in a repeated measures format. The data characterizes the generality or modulatory nature of the candidate compounds for ameliorating the effects of the drugs of abuse on dopamine signaling pathways.

Third, plethysmography is used to examine whether SBI-553 and IND ready analog SBI-810 can be administered to mice intraperitoneally, intravenously, or intrathecally without respiratory depression. Using plethysmography determine whether SBI-553 and IND ready analog SBI-810 can be administered to mice (e.g., C57BL6J) intraperitonially, intravenously, or intrathecally without respiratory depression. SBI-553 did not exacerbate the reduction in breathing frequency or tidal volume produced by morphine and had no effect on its own). The “Buxco FinePointe Whole Body Plethysmography 4-Site System” was used to assess whether any IND analogs like the SBI-810 in the same system following intraperitoneal, intravenous, or intrathecal administration. Here. C57BL6J mice are assessed using UWBP, a technique that provides a, non-invasive, quantitative means to assess and analyze respiratory parameters. This will include tidal volume, minute volume, inspiratory duty cycle, inspiratory flow rate and the ratio of inspiration time to expiration time using a repeated measures approach to determine lung function on the same animal under multiple treatment conditions.

Fourth, cellular assays are used to establish that IND ready analogs like SBI-810 are still selective for the NTSR1 rather than NTSR2. The specificity of the lead compounds toward NTSR1. NTSR2. and β-arrestin-2 are tested in respective cellular assays. More specifically, cells expressing either NTSR1 or NTSR2 are used to assess the function of various compound. When activated by SBI-553 or SBI-810. cells expressing NTSR1 demonstrate translocation of β-arrestin-2 to intracellular compartments. This translocation is not observed in cells expressing NTSR2.

As it concerns to various models of pain in mice and rats, a variety of approaches is used to verify that the β-arrestin biased NTSR1 ligand SBI-553 and SBI-810 compound retain their selectivity and function. Assays for assessing β-arrestin signaling including readouts based upon protein complementation, reporter bioluminescence resonance energy transfer (BRET), and fluorescence are all based on the original data. For G protein signaling, a wide variety of radioligand, luminescence, fluorescence and imaging assays are employed in both in multi-well plate and non-plate format. For instance, a bioluminescence proximity reporter for measurements of cAMP production, a mitochondria-localized aequorin reporter calcium mobilization assay for GPCRs, is used. A series of surrogate cellular assays, based on binding and biochemical profile that report the pharmacological action of a GPCR ligand such as SBI-553. are employed. In addition to the cellular assays, additional ways to validate SBI-553 and analogs thereof that involve behavioral paradigms in animals are used.

Fifth, the specificity of the lead compounds against NTSR1, NTSR2, and β-arrestin-2 is also tested in respective knockout mouse lines (NTSR1, β-arrestin-2, and NTSR2).

Any optimized analogs of SBI-553 recapitulate the pharmacological and physiological properties of SBI-553. Therefore, as described above, any newly develop structural analog of SBI-553 necessitate the comprehensive testing as demonstrated above of any new potential ligands. Data, unless otherwise specified, are presented as mean±SEM and are acquired through at least 3 independent experiments. When a difference between group means is observed, the significance between groups is determined using analysis of variance incorporating standard post hoc testing and analysis. For fitting of nonlinear models to data, the extended sum of squares F-testing is used via as employed by GraphPad Prism software version 7. While the SB compounds are highly selective for NTSR1 in cellular assays, the involvement of NTSR2 cannot be uniformly discounted. Notably, NTSR2 is widely expressed in the nociceptive system, including DRG neurons and contributes to NT-induced analgesia (Kupari J, et al. (2021) Nat Commun. 12(1):1510). Thus, low activity on NTSR2 may enhance the analgesic effect of the SB compounds. Thus, it is important to genetically engineer a NTSR2 knockout mouse line as a previously described NTSR2KO mouse line is no longer available (Oliveros A, et al. (2010) J Exp Biol. 213(Pt 24):4232-4239). Such a line is useful to functionally characterize the specificity of the NTSR1 ligands.

### Example 2

### Determination of Pharmacokinetic Properties and Acute Toxicity of Lead Compounds in Mice

As shown in FIG. 8, the pharmacokinetics properties of SBI compounds have been assessed in mouse, rat, and monkey following plasma concentration only after either IV or PO administration. The evaluation of the effects of these various compounds using additional routes of administration and optimize dosing based on MTD and PK parameters for each route (see below) is performed. The examination of drug distribution in the pain pathway (e.g., DRG, SC) is performed. Several tasks are performed in this determination.

First, plasma and microsomal stability of lead compounds are investigated. The knowledge of plasma stability at room temperature is a prerequisite for assay development and plasma sample handling protocol. In-vitro (37° C.) plasma and microsomal stability is critical for proper interpretation of PK profiles. Plasma and microsomal media are fortified with the disclosed lead compounds and are incubated at either RT (plasma) or 37° C. (plasma and microsomes). At predetermined time-points, a LC/MS/MS assay is used to quantify the remaining drug in the sample. These data provide on-bench and in-vitro stability of plasma and the intrinsic metabolic clearance by liver.

Second, the maximum tolerable dose (MTD) of lead compounds is measured. It is important to determine the maximum tolerable dose of lead compounds so as to prolong the desirable maximum therapeutic effects of lead compounds. Mice and rats are treated with increasing bolus concentrations of either orally (PO), subcutaneously (SC), intravenously (IV). and intrathecally (IT) and the mice and rats are observed for unacceptable side effects. Single-dose (24 hr) MTD and multi-dose (7 days) MTD experiments are performed and the resulting obtained doses are integrated into the process for optimal design of PK/PD and efficacy studies. Generally, the number of mice used to determine the MTD is about 72 (e.g., 9 mice×2 drugs×4routes). Generally, the number of rats used to determine the MTD is about 72 (e.g., 9 rats×2 drugs×4 routes).

Third, the pharmacokinetic (PK) study of lead compounds, administered orally. subcutaneously, intravenously and intrathecally is evaluated. FIG. 8 shows that PD/PK studies of lead compounds in mouse and rats for SBI-553 and monkey for both SBI-553 and SBI-810. As shown in the graphic images values of plasma concentration over time in monkey after either IV (left) or PO dosing (right). FIG. 8 shows that both SBI-553 and SBI-810 have excellent properties as pharmacological agents. To maximize the likelihood of success of the lead compounds for maximal therapeutic effects, the search for optimal route of administration is expanded by adding subcutaneous (SC) and intrathecal (IT) routes along with intravenous (IV) and oral (PO) routes.

Accumulating evidence indicates that the blood-brain barrier/blood-spinal cord barrier (BBB/BSCB) in the CNS, as well as peri-neural barrier of the sciatic nerve in the PNS, might be disrupted in neuropathic conditions. This leads to neuroinflammation and altered drug availability in the CNS and PNS (DosSantos M F, et al. (2014) Front Cell Neurosci. 8:302: Beggs S, et al. (2010) Mol. Pain. 6:74: Ji R R, et al. (2014) Nat. Rev. Drug Discov. 13(7):533-548). Thus, BBB permeation and tissue distribution of these biased agonists, are measured both in naive mice and mice treated with chemotherapy (2 weeks after PTX injections).

Plasma is collected from each animal, PK properties are examined following PO, SC, IV, and IT administration after single dose and using single-mouse serial blood draw methodology optimized for obtaining sufficient and quality PK information with minimal number of animals. Four (4) mice per route are administered single MTD and blood is drawn from tail at least the following time-points—5 min, 10 min, 30 min, 1 hr, 3 hr, 8 hr, and 24 hr. Plasma is separated and drug levels are measured by LC/MS/MS assay specifically optimized for small sample size and high sensitivity. The obtained conc./time data is used for non-compartmental calculation of PK parameters using WinNonlin software. The maximal plasma exposure parameters (Cₘₐₓ, AUC), low clearance (CL), and prolonged mean residence time (MRT) are determined for each route. The number of mice used in the plasma PK studies is 32 (e.g., 4 mice×2 drugs×4 routes×1 dose).

Both SBI-553 and SBI-810 are systematically administrated (IV, 12 mg/kg) and tissues are collected at 1 hr, 3 hr, 8 hr, 24 hr, 48 hr, and 72 hr after the drug injection. Four (4) tissue samples are harvested from each animal: (i) sciatic nerves, (ii) DRGs (L3-L6), (iii) spinal cord, and (iv) brain. The number of mice used to evaluate tissue PK is 144 mice (6 mice×2 drugs×1 route×1 dose×2 conditions×6 time points), which generates 720 samples. In total. 176 mice are used to generate 752 samples. Thus, the total number of mice used in the plasma and tissue analyses is about 176 mice and the total number of samples is about 752 samples.

Fourth, genetically engineered mice are used to assess the effects and mechanisms of novel ligands. The specificity of the lead compounds toward NTSR1, NTSR2, and β-arrestin-2 dependent signaling is examined. More specifically, cells expressing either NTSR1 or NTSR2 are used to assess the function of various compounds. Activation by SBI-553 or SBI-810 in cells expressing NTSR1 leads to the translocation of β-arrestin-2 to intracellular compartments whereas this response should not occur in cells expressing NTSR2. However, to directly confirm these observations, a knockout of the NTSR2 in mice is genetically engineered. The profiles of the identified SBI compounds indicates that both compounds are amenable to as possible treatment to relive the various forms of pain. Interestingly, the abilities of pharmacological agents to relive pain is often dictated by their relatively long half-life and their stability in the context of physiological fluids. Thus, the properties of the SBI compounds are consistent with their being able to act as potential effective pain medications.

### Example 3

### Determination of the Analgesic Effects of Lead Compounds in Mice Models of Acute and Chronic Pain and Rat Model of Neuropathic Pain

Given the increased appreciation of sex dimorphism in pain regulation (Mogil J S, et al. (2020) Nat Rev Neurosci. 21(7):353-365: (Sorge R E, et al. (2015) Nat. Neurosci. 18(8):1081-1083: Taves S, et al. (2016) Brain, behavior, and immunity, 55:70-81: Mogil J S. (2012) Nat. Rev. Neurosci. 13(12):859-866; Maixner W, et al. (1993) Clin J Pain. 9 (1): 16-25; Riley J L 3rd, et al. (1998) Pain, 74 (2-3): 181-187), both males and females are included in all the studies described below: Animals are randomly assigned to different groups. Based on preliminary data (Taves S, et al. (2016) Brain, behavior, and immunity. 55:70-81: Xu Z Z, et al. (2010) Nat. Med. 16 (5): 592-7. 1p: Xu Z Z, et al. (2015) Nature medicine. 21(11):1326-31; Chen G, et al. (2017) Nat Neurosci. 20(7):917-926). 10 mice (5 males and 5 females) are used for behavioral studies (tested blindly) and biochemical and histochemical studies to generate statistic power of >0.90. If sex differences are detected, then the data from both sexes is analyzed separately. The sample sizes for each experiment are based on preliminary data (Wang Z, et al. (2020) Sci Transl Med. 12 (531): Xu Z Z, et al. (2015) Nature medicine, 21(11):1326-31; Yang Q, et al. (2014) J Neurosci. 34(32):10765-10769; Wu Z, et al. (2017) J Neurotrauma. 34(6):1260-1270; Li G, et al. (2020) Neurosci Bull. 36(4):372-384). Normal distribution of the data is assessed with the D'Agostino-Pearson omnibus normality test, when applicable. Non-parametric or Log transformation approaches are considered when the data are non-normally distributed. All the data are expressed as mean # SEM, as in preliminary studies, or as mean +SD in some cases when necessary. Biochemical and behavioral data are analyzed using unpaired or paired two-tailed Student's t-test (two groups), One-Way ANOVA, or Two-Way ANOVA followed by post-hoc Bonferroni test. Statistical significance is identified by a p<0.05.

In these tasks, drugs are given by intrathecal injection (IT, 1-10 mg), IP injection, or oral administration (1-30 mg/kg). A sample size of 10 animals (e.g., 5 males and 5 females) are included. A statistician assists with experimental design and data analysis. Both evoked pain (mechanical and thermal sensitivity) and spontaneous pain such as guarding behavior and conditioned placed preference (CPP) are tested in a blinded manner.

As described below, three (3) mouse models of acute pain and neuropathic pain and one rat model of neuropathic pain are tested. Four (4) different local and systemic administration routes are used: (i) intrathecal (i.t.) route as shown in the preliminary work (FIG. 10A-FIG. 10E) to test the spinal/central action, (ii) intraplantar (i.pl.) route to test peripheral action, (iii) systemic IP route to target both peripheral and central mechanisms, and (iv) oral administration (p.o.) that has higher clinical relevance and suitable for SBI-810.

SBI-553, SBI-810, and vehicle (10% DMSO) are administered at different time points of injury (see below). The dose selection (see below) is based on the pilot studies and are optimized during the course of this project, if necessary. C57BL/6 mice of both sexes (n=5 males+n=5 females, 8-12 weeks) are used for behavioral tests that are performed in a blinded manner. Mechanical sensitivity is tested by von Frey fibers and assessed as PWT and paw-withdrawal frequency (PWF). Cold sensitivity is assessed using acetone test. Loss of function (tactile function) in the CIPN models is assessed by the tape test. Operant measurement such as conditioned place preference (CPP) and conditioned place aversion (CPA) tests are also be performed to assess ongoing pain. Guarding behavior is used to assess spontaneous pain after incision (Yamakita S, et al. (2017) Neuroscience. 361:58-68). Motor function are evaluated in Rotarod testing. Pain-associated comorbidities (e.g., depression and anxiety) in the late-phase (>4 weeks) after SNI and CIPN are also examined in open-field and elevated plus maze testing. Several tasks are performed in this determination.

First, acute pain is produced by paw incision to mimic postoperative pain (Brennan T J, et al. (1996) Pain. 64(3):493-502). This model is highly relevant to military personnel with acute injury involving skin, muscle, and tendon. It is also relevant to military personnel who need acute postoperative pain management. The effect of treatment with SBI-553. SBI-810, and vehicle are examined in three different pain models (e.g., with n=10 mice/group of which 5 are males and 5 are female). The total number of mice is about 540) (e.g., 3 treatment groups×3 models×3 routes×2 doses×10 mice/group). A mouse chemotherapy model of paclitaxel (PTX) is employed. Chemotherapy-induced peripheral neuropathy (CIPN) occurs in up to 80% patients with chemotherapy and has significant impact on patient function and health-related quality of life (Sisignano M, et al. (2014) Nat Rev Neurol. 10(12):694-707). Animal model of CIPN will be induced by the chemotherapy agent paclitaxel (4×2 mg/kg, i.p., given on day 0, 2, 4, and 6). This model administers SBI-553. SBI-810, or vehicle via an i.t, injection (1 nmol and 10 nmol) and s.c. injection (12 mg/kg and 30 mg/kg) at 2 weeks (early-phase) and at 4-8 weeks (late-phase) after the first PTX injection. Neuropathic pain (mechanical and cold allodynia) is tested at 1 hr, 3 hr, 5 hr, and 24 hr after a single injection (FIG. 10A). Alternatively, this model uses an oral route of administration (PO, 12 mg/kg) given daily for one week at 2 weeks and 4 weeks. Neuropathic pain (mechanical and cold allodynia) is tested on day 1, 3, 5, 7, 10 after weekly oral treatment. CPP/CPA and Rotarod tests are conducted 1-2 times in each animal.

A mouse SNI model is employed. This model administers SBI-553, SBI-810, or vehicle via i.t, injection (1 nmol and 10 nmol) and IP injection (12 mg/kg and 30 mg/kg) at 2 weeks (early-phase) and 4 weeks (late-phase) after nerve injury. Alternatively, this model uses an oral route of administration (12 mg/kg) given daily for one week at 2 weeks and 4 weeks. Neuropathic pain (mechanical and cold allodynia) is tested at 1 hr, 3 hr, 5 hr, and 24 hr after a single injection. Neuropathic pain (mechanical and col allodynia) is tested on day 1, 3, 5, 7, 10 after weekly oral treatment. CPP/CPA and Rotarod tests are conducted 1-2 times in each animal.

A mouse plantar incision model is employed. Plantar surgical pain is produced by hind paw incision (Brennan T J, et al. (1996) Pain. 64(3):493-501). This model is highly related to surgery as well as battlefield injury as it involves not only skin and muscle injury but also tendon and small nerve branches associated with some degree of neuropathy. This model administers SBI-553, SBI-810, or vehicle via an i.t, injection (1 nmol and 10 nmol) and IP injection (12 mg/kg and 30 mg/kg) at 2 hr. Alternatively, this model uses an oral route of administration (12 mg/kg) given at 2 hr, 24 hr, and 48 hr. Postoperative pain (mechanical allodynia, guarding behavior) is tested at 1 hr, 3 hr, 5 hr, and 24 hr after a single injection. Postoperative pain is tested on day 1.3, and 5 after oral treatment. CPP/CPA and Rotarod tests are conducted for each animal.

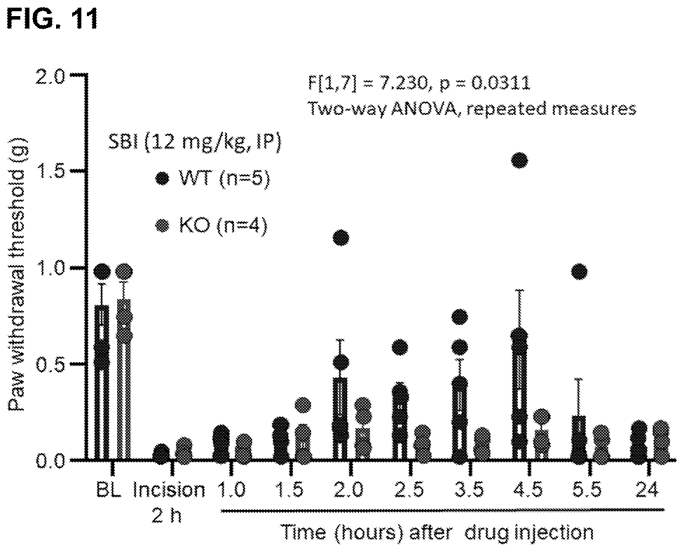

Second, chronic pain (neuropathic pain) is induced by chemotherapy (paclitaxel model) and nerve injury (spared nerve injury, SNI model) (Chen G, et al. (2016) Nat Commun. 7:12531; Decosterd I, et al. (2000) Pain. 87(2):149-158). Arrb2 regulates TRPV1 and NMDAR activity via desensitization (Chen G, et al. (2016) Nat Commun. 7:12531; Por E D, et al. (2012) J Biol Chem. 287(44):37552-37563; Caterina M J, et al. (2001) Annu. Rev. Neurosci. 24:487-517). The preliminary data showed that systemic injection of SBI-553 (12 mg/kg. IP) significantly reduced plantar incision-induced mechanical allodynia in WT but not in Arrb2 KO mice (n=4-5). Notably, the analgesic effect was observed 2 hr after the injection and lasted for more than 4.5 hr in this surgery pain model (FIG. 11). Whether a biased agonist can inhibit the capsaicin and NMDA-induced pain is examined using several different models.

First, a model of intrathecal NMDA-induced pain in mice is used. Here, an i.t, injection of NMDA (1 nmol) produces a rapid spontaneous pain for 10 min and mechanical allodynia for 1-2 weeks (Chen G, et al. (2016) Nat Commun. 7:12531). An i.t. (1 nmol and 10 nmol) or IP injection (12 mg/kg and 30 mg/kg) of SBI-553, SBI-810, or vehicle is provided at 1 hr prior to the NMDA injection and 7 hr after the same NMDA injection. NMDA-induced spontaneous pain is tested in the first 10 min (FIG. 5C) and NMDA-induced mechanical allodynia is tested be tested at 1 hr, 3 hr, 5 hr, and 24 hr.

Second, a model of intraplantar capsaicin-induced pain in mice is used. Here, an i.pl. injection of capsaicin (10) mg) evokes spontaneous pain in the first 5 min and also evokes primary and secondary mechanical allodynia for up to 6 hr (Chen G, et al. (2016) Nat Commun. 7:12531). An i.pl, injection (1 nmol and 10 nmol) and s.c, injection (12 mg/kg and 30 mg/kg) of SBI-553 or SBI-810 is administered 1 hr prior to the capsaicin injection. Capsaicin-induced spontaneous pain is assessed in the first 5 min, and the capsaicin-induced mechanical allodynia is tested be tested at 1 hr, 2 hr, 4 hr, and 6 hr.

In these two different pain models experiments. 36 groups of animals (n=10 mice/group. 5 males and 5 females) are used for the treatment of SBI-553, SBI-810, and vehicle. Thus, total number of animals is 240 mice (e.g., 3 drugs/vehicle×2 models×2 routes×2 doses×10 mice/group). The sample size in these experiments, however, can be increased to n=10 for both sexes.

Third, the lead compounds are evaluated using a rat model of neuropathic pain induced by SNI. Spared nerve injury (SNI) results in long-lasting neuropathic pain for >6 months (Decosterd I, et al. (2000) Pain. 87(2):149-158). Thus, to enhance the translational feasibility, a rat model of SNI (Wen Y R, et al. (2007) Anesthesiology. 107(2):312-321; Tao X, et al. (2021) Front Physiol. 12:687046) is used. An i.t, injection (2 nmol and 20 nmol) and an IP injection (12 mg/kg and 30 mg/kg) of SBI-553, SBI-810, or vehicle is administered at 2 weeks (early-phase) and 8 weeks (late-phase) after SNI nerve injury. Oral administration (12 mg/kg) is administered daily for one week at 2 weeks and 8 weeks. Neuropathic pain (mechanical and cold allodynia testing) is tested at 1 hr, 3 hr, 5 hr, and 24 hr after a single injection on days 1, 3, 5, 7, and 10 after weekly oral treatment. CPP/CPA, open field, are Rotarod tests are performed. In these experiments, 180 rats are used (e.g., 3 drugs/vehicle×1 model×3 routes×2 doses×10 rats/group).

The i.t, administration of SBI-553 is highly effective in reducing PTX-induced mechanical allodynia. Similar effects in the SNI and plantar incision models are observed, which confirms a central action of the biased agonists. These results support a broad effect of pain relief of these biased agonists in different models of neuropathic pain. The demonstration of efficacy in treating postoperative/surgical pain is highly significant as this type of acute pain occurs frequently in military personnel. Notably, the FDA approved the biased agonist Olinvyk for IV short-term treatment of acute surgical pain in a hospital setting.

The analgesic effect of local i.pl, injection of the SBI compounds in the capsaicin model is detected, indicating that the biased agonist also has a peripheral action. This is a novel finding for the biased agonists.

Systemic (IP or oral) administration of SBI compounds inhibit pain via both central and peripheral action as these SBI compounds are CNS-permeable. Notably, SBI-810 is a modified form of SBI-553 with enhanced oral availability, and therefore, is more effective in reducing pain than SBI-553. These SBI compounds are effective in alleviating pain in both sexes. Similar analgesic actions of the biased agonists are effective in mice and rats as both species share similar molecular and cellular mechanisms in pain modulation.

### Example 4

### Determination of the Mechanisms by which the Lead Compounds Suppress Pain in Mice

Several tasks are performed in this determination.

First, whether the lead compounds inhibit pain through NTSR1 using WT, NTSR1-KO, and NTSR2-KO mice is examined. Neurotensin suppresses pain via both NTSR1 and NTSR2 receptors, which are expressed by nociceptive neurons in the spinal cord and DRG (Zhang X, et al. (1995) J Neurosci. 15(4): 2733-2747; Yaksh T L, et al. (1982) Ann N Y Acad Sci. 400:228-243). Whether the lead compounds (SBI-553 and SBI-810) inhibit pain through these receptors using WT and NTSR1-KO mice (available) or NTSR2-KO (which are genetically engineered). 12 groups of animals are used (n=10 mice/group, 5 males and 5 females) for the treatment of SBI-553, SBI-810, and vehicle in one pain model. Thus, the total number of mice used is about 120 (3 drug/vehicle groups×1 model×1 route×1 dose×4 genotypes×10 mice/group).

Preliminary data show the efficacy of SBI-553 (12 mg/kg. IP) in blocking surgery-induced pain (FIG. 11). To this end, SBI-553, SBI-810, or vehicle are administered via IV (5-10 mg/kg) at 2 hr after hindpaw incision in WT and KO mice. Post-operative pain (mechanical allodynia, guarding behavior) is tested at 0.5 hr, 1 hr, 1.5 hr, 2 hr, 3 hr, 4 hr, 6 hr, and 24 hr after single injection, and then again on days 1, 3, 5, and 7.

Here, CPP/CPA, open field, and Rotarod tests are conducted for each animal. Moreover, these experiments use von Frey, hot plate, and tail flick tests to examine whether the baseline pain is affected in NTSR1-KO and NTSR2-KO mice.

Second, whether the lead compounds inhibit pain through β-arrestin-2 using WT and β-arrestin-2-KO mice is examined. β-arrestin-1 and β-arrestin-2 are two of the major isoforms of arrestins. Notably, morphine analgesia is potentiated in mice lacking Arrb2 (Chen G, et al. (2016) Nat Commun. 7:12531: Bohn L M, et al. (1999) Science. 286(5449):2495-2498). Whether the lead compounds (SBI-553 and SBI-810) inhibit pain through these receptors is examined using WT and Arrb1-KO and Arrb2-KO mice. SBI-553, SBI-810, or vehicle (IV, 5-10 mg/kg) is administered at 2 hr after hind paw incision in WT and KO mice. Post-operative pain (mechanical allodynia, guarding behavior) is e tested at 0.5 hr, 1 hr, 1.5 hr, 2 hr, 3 hr, 4 hr, 6 hr, and 24 hr after single injection, and then on days 1, 3, 5, and 7.

Here, CPP/CPA, open field, and Rotarod tests are conducted for each animal. Moreover, these experiments use von Frey, hot plate, and tail flick tests to examine whether the baseline pain is affected in Arrb1-KO and Arrb2-KO mice.

In this pain model, 12 groups of animals (n=10 mice/group, 5 males and 5 females) for the treatment of SBI-553, SBI-810, and vehicle. The total number of mice is 120 (e.g., 3 drug/vehicle groups×1 model×1 route×1 dose×4 genotypes×10 mice/group).

Third, whether the lead compounds inhibit pain through suppressing NMDAR function and spinal cord pain processing using electrophysiology in spinal cord slices is examined.

Calcium imaging is typically used to examine nociceptor activation (Kim Y S, et al. (2014) Neuron. 81(4):873-887). Advillin is a specific marker for sensory neurons, and Advillin-GCaMP6 GFP reporter mice was used to assess Ca²⁺ signal in primary sensory neurons (Wang K, et al. (2020) J Clin Invest. 130(7):3603-3620). The effects of the biased agonists on capsaicin-induced calcium influx are assessed. Ten (10) reporter mice (Advillin-GCaMP6 GFP reporter mice (n=10 mice/group, 5 males and 5 females) are used to prepare 2˜3 cultures per each animal (e.g., vehicle, SBI-553, and SBI-810 (1-30 mM, 2-5 min)).

Fourth, using the lead compounds to test the mechanisms that inhibit pain through suppressing nociceptor excitability in human DRG neurons. Non-diseased human DRGs will be obtained from the National Disease Research Interchange (NDRI).

Tissue injury-induced spinal cord synaptic plasticity (i.e., central sensitization) contributes importantly to the development and maintenance of chronic pain (Ji R R, et al. (2003) Trends in neurosciences. 26(12):696-70; Kuner R (2010) Nature medicine. 16(11):1258-1266; Park C K, et al. (2011) J Neurosci. 31(42):15072-15085). The hypothesis that the biased agonists inhibit synaptic plastic changes after CIPN is evaluated. In this experiment, 20 WT mice (10) males and 10 females) that are treated with PTX. Spinal cord slices (400 mm, 2-3/mice) are prepared 1-2 weeks after PTX treatment. For each animal, 2-3 neurons are tested. Patch clamp recordings in superficial dorsal horn neurons to record spontaneous post-synaptic currents (EPSCs) as previously described (Park C K, et al. (2011) J Neurosci. 31(42):15072-15085; Xu Z Z, et al. (2013) Annals of neurology. 74(3):490-495). NMDA (50 mM)-induced inward currents are also recorded to examine the effects of the SBI compounds. Lamina IIo neurons in the dorsal horn are at the focus at these experiments because these IIo neurons form a nociceptive circuit by receiving C-fiber input from TRPV1+ neurons and making synapses to projection neuron (Todd A J (2010) Nat. Rev. Neurosci. 11(12):823-836; Braz. J, et al. (2014) Neuron. 82(3):522-536). EPSCs are analyzed before and after treatment with vehicle, SBI-553, and SBI-810 (1-30 mM, FIG. 10C), and 8-12 neurons (3-5 mice) are included per group.

Fifth, whether the biased agonists can suppress nociceptor excitability in human DRG neurons from non-diseased donors is examined.

A “human neuropathic pain model in a dish” was established. PTX chemotherapy treatment (1 mM, 24 h) increased action potential firing frequency in human DRG nociceptor neurons (Chang W, et al. (2018) Neurosci Bull. 34(1):4-12). Using this human disease model, whether SBI-553 and SBI-810 (1-30 mM, 2 min-5 min) can block neuronal hyperexcitability in human DRG neurons induced by PTX (1 mM, 24 hr) was examined. Action potential firing rate and rheobase are examined to determine neuronal excitability after the treatment. Non-diseased human DRGs are obtained from donors through National Disease Research Interchange with permission from Duke IRB. Postmortem L3-L5 DRGs were obtained from both sexes with a wide age range (20 years-72 years) (Xu Z Z, et al. (2015) Nature medicine. 21(11):1326-31: Chang W, et al. (2018) Neurosci Bull. 34(1):4-12). A total of 12-20 neurons (6 DRGs/6 donors) are included for each condition. Single-cell analysis reveals both NTSR1 and NTSR2 mRNA expression in monkey nociceptor neurons (Kupari J, et al. (2021) Nat Commun. 12(1):1510). To confirm NTSR1 and NTSR2 mRNA expression in human DRG neurons, in situ hybridization using highly selective and sensitive RNAscope method (as previously described) (Luo X C O, et al. (2021) Neuron. In press.) is used.

The analgesic effects of the biased agonists are significantly reduced in KO mice lacking NTSR1 and Arrb2, indicating that the biased agonists signal through the NTSR1/Arrb2 pathway.

SBI compounds have significant inhibition on calcium signaling in DRG neurons and excitatory synaptic transmission (EPSC) in SDH neurons, indicating the involvement of both peripheral and central mechanisms. Since activation of Arrb2 suppresses NMDAR function, SBI compounds also suppress the NMDA-induced currents. Arrb 2-biased agonists relieve pain in part via NMDAR signaling.

Since activation of Arrb2 suppresses TRPV1 function, an inhibition of capsaicin-induced nociceptor activation by the biased agonists is examined using a calcium imaging study.

An inhibitory effect of the biased agonist in human nociceptor neurons is examined using an in vitro model for “human pain in a dish” (Chang W, et al. (2018) Neurosci Bull. 34(1):4-12). NTSR1/NTSR2 expression in human DRG neurons is identified, which indicates a clinical relevance of the pathway.

Increasing evidence indicates that non-neuronal cells, such as immune cells and glial cells contribute to chronic pain (Ji R R, et al. (2016) Science. 354(6312):572-577). Spinal cord, DRG, and blood tissues from the treated mice are collected to examine inflammatory and neuro-inflammatory markers (cytokines and chemokines) and glial reaction after the treatment.

The behavioral and physiological effects of NTSR1 have previously been attributed to NTSR1 Gq activation. But, as described herein, the role that selective activation of NTSR1 β-arrestin signaling, in the absence of Gq signaling, in changing DRG physiology and pain behaviors is surprising. To this end, the ability to use the selecdtive NTSR1 β-arrestin ligands disclosed herein in an epidural injection in a hospital setting (e.g., for surgery and/or baby delivery) and intra-articular treatment (e.g., for arthritic pain and low-back pain) demonstrate the significance of the claimed compositions and methods.

Ultimately, the primary significance of using a disclosed composition or a disclosed pharmaceutical formulation in a disclosed method of treating and/or preventing pain lies in the ability to reduce the use of opioid pain medication (e.g., either fully or partially through co-administration). The disclosed compositions and the disclosed pharmaceutical formulations represent the first use of β-arrestin biased NTSR1 ligands to treat and/or prevent pain.

1.-15. (canceled)

16. A method of treating and/or preventing pain in a subject, the method comprising:
administering to a subject in need thereof a therapeutically effective amount of a composition comprising a neurotensin receptor 1 (NTSR1) ligand having functionally selective β-arrestin biased activity or a pharmaceutical formulation thereof,
wherein pain is alleviated and/or minimized in the subject, and
wherein the ligand does not elicit hypothermia, hypotension, and/or motor impairment in the subject.

17. The method of claim 16, wherein the therapeutically effective amount of the NTSR1 ligand comprises a dose of about 1 mg/kg/day to about 100 mg/kg/day.

18. The method of claim 16, further comprising repeating the administering of the composition or the pharmaceutical formulation to the subject.

19.-20. (canceled)

21. The method of claim 16, wherein the subject has acute pain, chronic pain, or both.

22. The method of claim 16, wherein the subject has one or more bone fractures.

23. The method of claim 22, wherein the one or more bone fractures is the result of a non-trauma, a disease, or a disorder.

24. The method of claim 16, wherein the subject has sustained one or more injuries and/or one or more physical injuries.

25. The method of claim 16, wherein the subject has experienced one or more surgical procedures.

26. The method of claim 16, wherein the subject has an opioid dependence.

27. The method of claim 16, further comprising improving the subject's ability to move and/or the subject's level of mobility.

28. The method of claim 16, further comprising administering to the subject a therapeutically effective amount of one or more therapeutic agents.

29. The method of claim 28, wherein the one or more therapeutic agents are administered to the subject prior to, concurrently, or after the administration of the composition or the pharmaceutical formulation.

30. The method of claim 28, wherein the one or more therapeutic agents comprises a STING agonist, a steroid, an opioid analgesic, a local anesthetic, an NSAID, a programmed cell death 1 ligand, an anti-cancer agent, and any combination thereof.

31. The method of claim 30, wherein the opioid analgesic comprises codeine, dextropropyoxyphene, diamorphine, dihydrocodeine, meptazinol, methadone, morphine, nalbuphine, pentazocine, oxycodone, and any combination thereof.

32. The method of claim 28, further comprising repeating the administering of the one or more therapeutic agents.

33. The method of claim 28, further comprising monitoring the subject for adverse effects.

34. The method of claim 33, wherein in the presence of adverse effects, the method further comprises modifying an aspect of the method.

35. The method of claim 34, wherein modifying an aspect of the method comprises changing the amount of the composition, the pharmaceutical formulation, and/or the therapeutic agent administered to the subject, changing the frequency of administration of the composition, the pharmaceutical formulation, and/or the therapeutic agent to the subject, changing the duration of time that the composition, the pharmaceutical formulation, and/or the therapeutic agent is administered to a subject, or any combination thereof.

36. (canceled)

37. The method of claim 16, wherein the NTSR1 ligand having functionally selective β-arrestin biased activity comprises
(i) the general formula (I): 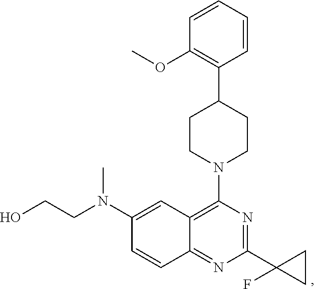
or a pharmaceutically acceptable salt, solvate, hydrate, prodrug, ester, or derivative thereof, or
(ii) the general formula (II): 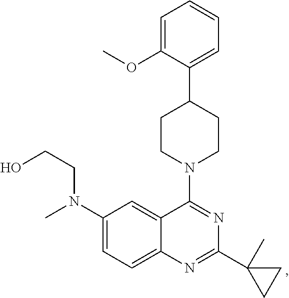
or a pharmaceutically acceptable salt, solvate, hydrate, prodrug, ester, or derivative thereof.

38. The method of claim 16, wherein the NTSR1 ligand having functionally selective β-arrestin biased activity is SBI-553 or SBI-810, or a pharmaceutically acceptable salt thereof.
